(19) United States
(12) Patent Application Publication
(10) Pub. No.: US 2018/0264241 A1
(43) Pub. Date: Sep. 20, 2018

(54) DEVICES AND METHODS FOR DELIVERING SUBTANCES FOR IMPROVED WELLBEING

(71) Applicants: Donald PLANCE, Montrose, CA (US); Alan BACKUS, Los Angeles, CA (US)

(72) Inventors: Donald PLANCE, Montrose, CA (US); Alan BACKUS, Los Angeles, CA (US)

(21) Appl. No.: 15/463,466

(22) Filed: Mar. 20, 2017

(51) Int. Cl. A61M 31/00; A61M 15/08; A61M 21/02; A61J 7/00

(52) U.S. Cl. CPC A61M 31/002; A61M 15/08; A61M 21/02; A61J 7/0053; A61M 2021/0016; A61M 2210/0662; A61M 2210/0618; A61M 2209/06; A61J 7/003

(57) ABSTRACT

A method and apparatus to deliver biological altering substances through body cavities including ears, nose, mouth, anus and vagina, utilizing at least osmotic, olfactory and inhaling transfusion of active ingredients includes a first reservoir configured to be disposed against sublingual surfaces and hold a first drug. The device is configured to transmit the first drug through sublingual surfaces into the bloodstream. A second reservoir, coupled to the first reservoir, is disposed below a nostril, and configured to hold a substance and transmit the substance into air passing from the exterior of the nostril into the nasal cavity.

### TECHNICAL FIELD

The present application is directed toward devices which deliver drugs and other substances to improve well-being into the human body.

### BACKGROUND

From a broad perspective, this field of devices to deliver drugs into the human body is very crowded and broad. It includes, just to name a few such devices: injections, oral tablets, suppositories, transdermal patches, medicated gums, lozenges, and candies; sublingual tablets, sprays and pads, subdermal implants, inhalers, vaporizers, pic lines, ports, dermal sprays, wraps, and many more.

The present applications are intended to introduce devices to this field which are both convenient to use, and effective.

### SUMMARY

The present application is intended for insertion into body cavities, including: ears, nose, mouth, anus, arm pits, and vagina. Embodiments may transfer substances using gases, vapors, suspensions, direct contact, or other means; and/or combinations of the foregoing.

Such transferred substances may alter human physical and/or mental functions and/or states, or provide other benefits.

### BRIEF DESCRIPTION OF THE DRAWINGS

Various embodiments will become better understood with regard to the following description, appended claims and accompanying drawings wherein:

FIG. 1 is a view of an individual using embodiment 102.

FIG. 2 is a view of an individual using embodiment 104.

FIG. 3 is a section taken through a human ear, showing embodiment 100 inserted into it.

FIG. 4 is a section taken through a human head showing embodiment 106 inserted into nostril 108.

FIG. 5 is a perspective of embodiment 110.

FIG. 6 shows perspective section 111, of embodiment 110 as indicated in FIG. 6.

FIG. 7 is a perspective of embodiment 112.

FIG. 8 shows perspective section 114 of embodiment 112 as indicated in FIG. 8.

FIG. 9 is a perspective of embodiment 116.

FIG. 10 shows perspective section 118, as indicated in FIG. 10.

FIG. 11 is a perspective of embodiment 120.

FIG. 12 shows perspective section 122, of embodiment 120, as indicated in FIG. 12.

FIG. 13 is a perspective of embodiment 124.

FIG. 14 shows perspective section 126 of embodiment 124, as indicated in FIG. 14.

FIG. 15 is a perspective of embodiment 128.

FIG. 16 shows perspective section 130, of embodiment 128, as indicated in FIG. 16.

FIG. 17 is a perspective showing packaging and/or preparation of one of the embodiments taken from the set of embodiments 110, 112, 116, 120, 124, and 128.

FIG. 18 is a perspective of preparation and/or packaging of one of the embodiments taken from the set of embodiments 110, 112, 116, 120, 124, and 128.

FIG. 19 is a perspective of preparation and/or packaging of one of the embodiments taken from the set of embodiments 110, 112, 116, 120, 124, and 128.

FIG. 20 is background corresponding to aroma therapy.

FIG. 21 is background corresponding to aroma therapy.

FIG. 22 is background corresponding to aroma therapy.

FIG. 23 is background corresponding to aroma therapy.

FIG. 24 is background corresponding to aroma therapy.

FIG. 25 is background corresponding to aroma therapy.

FIG. 26 is background corresponding to aroma therapy.

FIG. 27 is background corresponding to aroma therapy.

FIG. 28 is background corresponding to aroma therapy.

FIG. 29 is background corresponding to aroma therapy.

FIG. 30 is a perspective of embodiment 176 when it is unrolled.

FIG. 31 is a perspective of embodiment 176 when it is rolled up.

FIG. 32 is a cross-section of a human head while embodiment 176 is in use.

FIG. 33 is a perspective of embodiment 178.

FIG. 34 is an exploded perspective of FIG. 178.

FIG. 35 is an exploded perspective section of FIG. 34, as indicated in FIG. 34.

FIG. 36 is a cross-section of embodiment 178, as indicated in FIG. 33.

FIG. 37 is a cross-section of a human head with embodiment 178 in use.

FIG. 38 is a perspective of embodiment 180.

FIG. 39 is a perspective cross-section of embodiment 180, as indicated in FIG. 38.

FIG. 40 is an exploded perspective cross-section of embodiment 40 utilizing the same section plane as FIG. 39.

FIG. 41 is a perspective of embodiment 180 taken from below embodiment 180.

FIG. 42 is a cross-section of a human head with embodiment 204 inserted into nostril 206.

FIG. 43 is a perspective cross-section of embodiment 204.

FIG. 44 shows an individual while using embodiment 208.

FIG. 45 is a perspective of embodiment 208.

FIG. 46 is a cross-section of embodiment 208, as indicated in FIG. 45.

FIG. 47 is a cross-section of a human head with embodiment 208 in use.

FIG. 48 is a perspective exploded view of embodiment 208.

FIG. 49 is a perspective of embodiment 210.

FIG. 50 is a cross-section of a human head with embodiment 210 in use.

FIG. 51 is a perspective view of embodiment 212.

FIG. 52 is a perspective of embodiment 214.

FIG. 53 is a cross-section taken through embodiment 214 as indicated in FIG. 52.

FIG. 54 is a perspective of a non-exhaustive and non-limiting example packaging for either embodiment 212 or embodiment 214.

FIG. 55 is a cross-section taken through a human head showing either embodiment 212 or embodiment 214 in use.

FIGS. 56 through 158 show non-exhaustive and non-limiting possibilities of products containing active ingredients which might be used in embodiments shown herein.

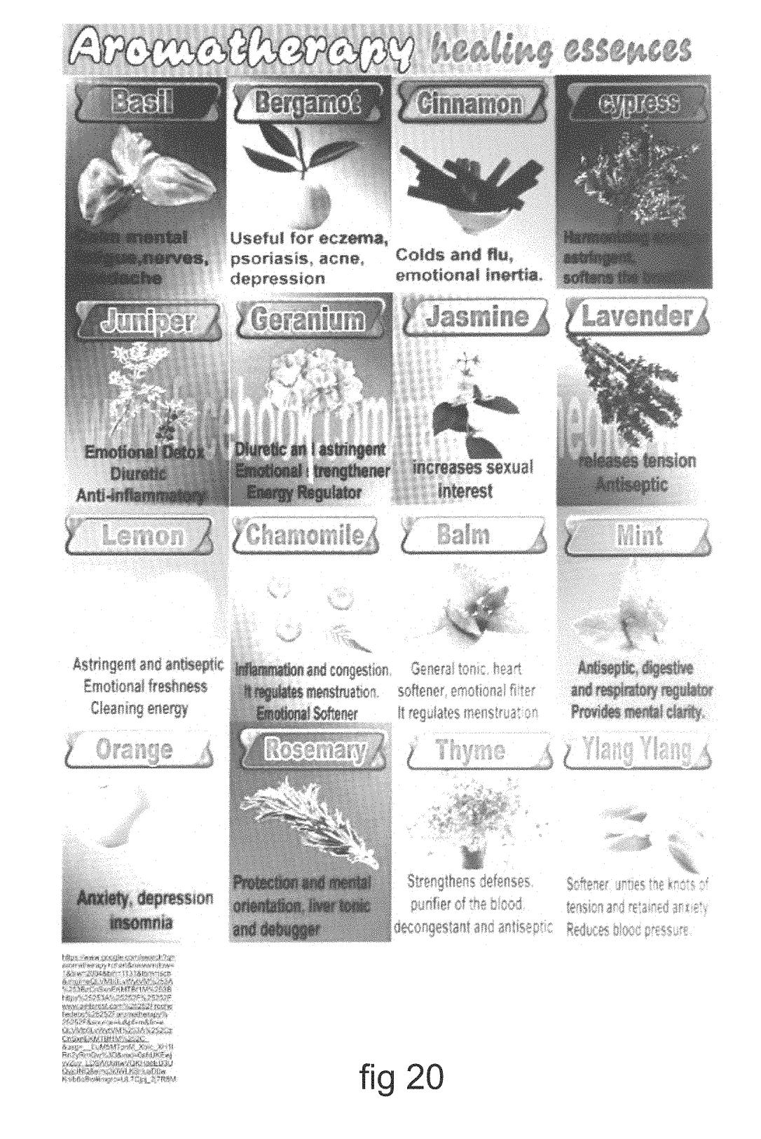

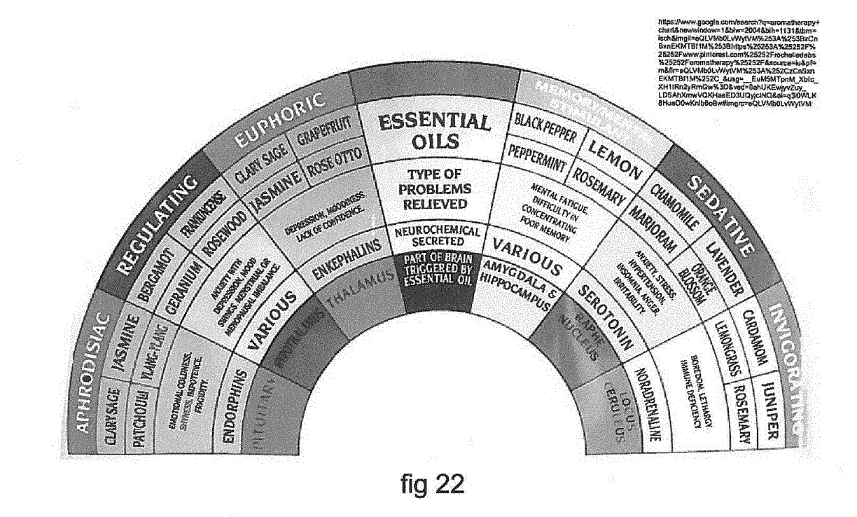

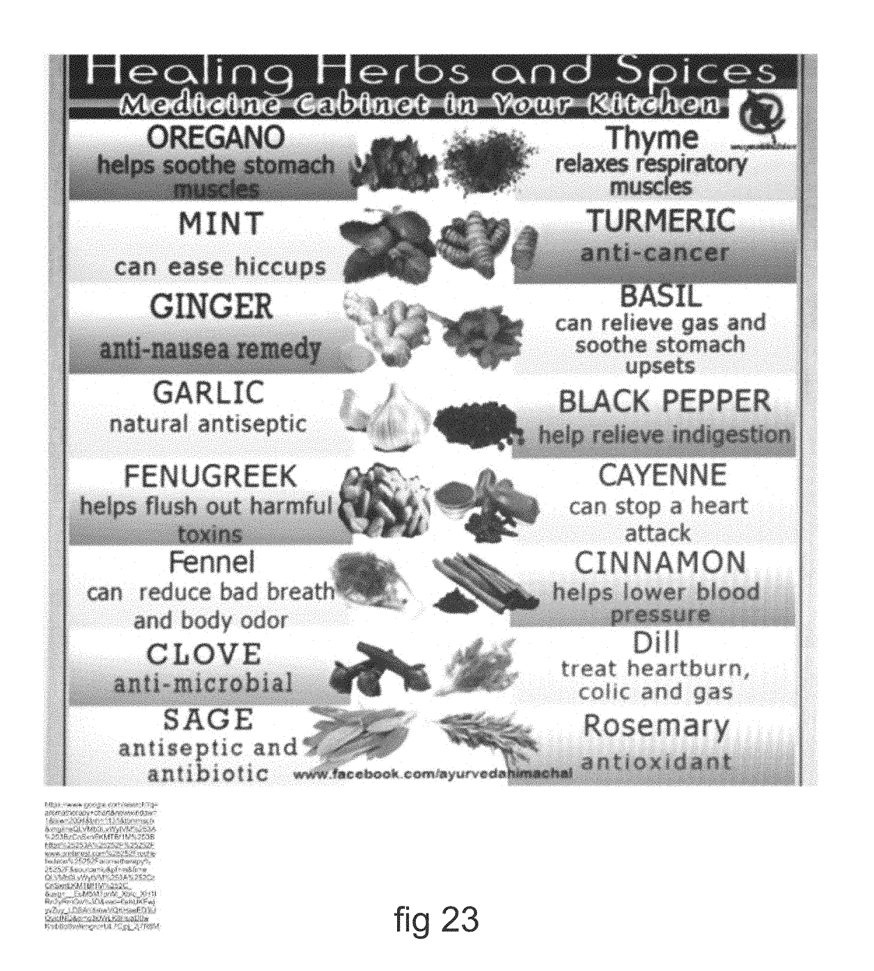

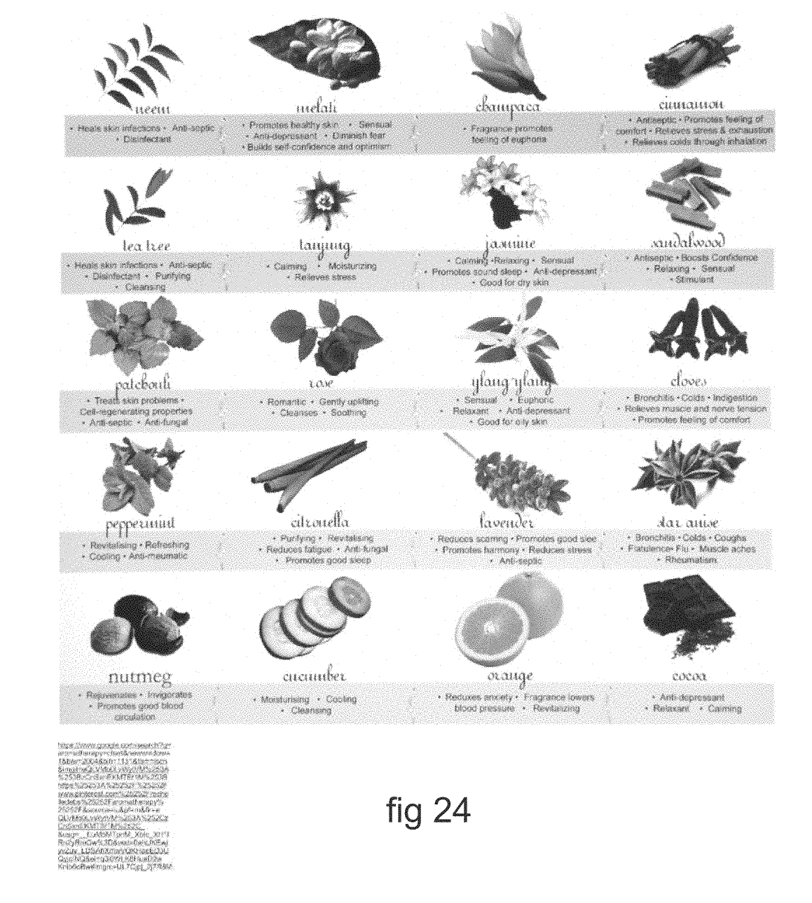

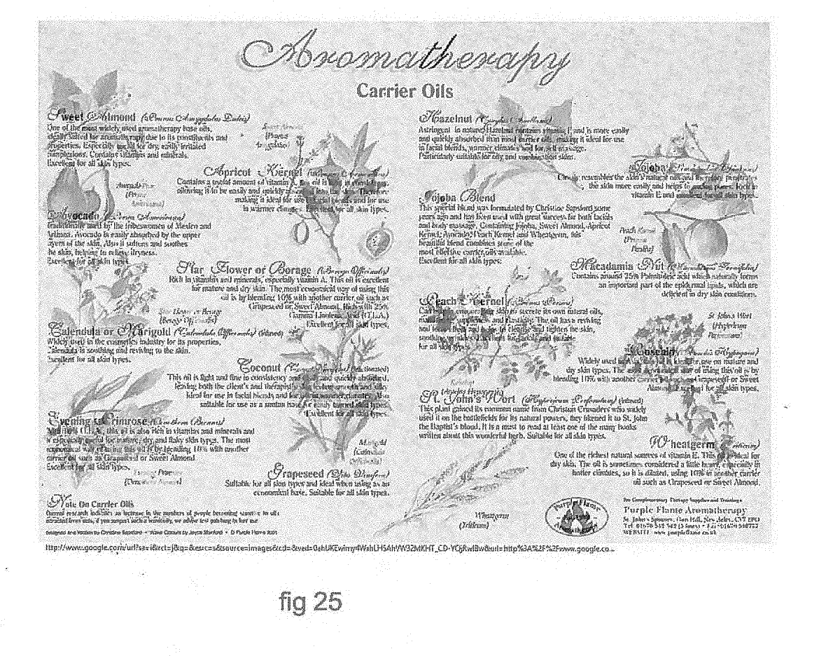

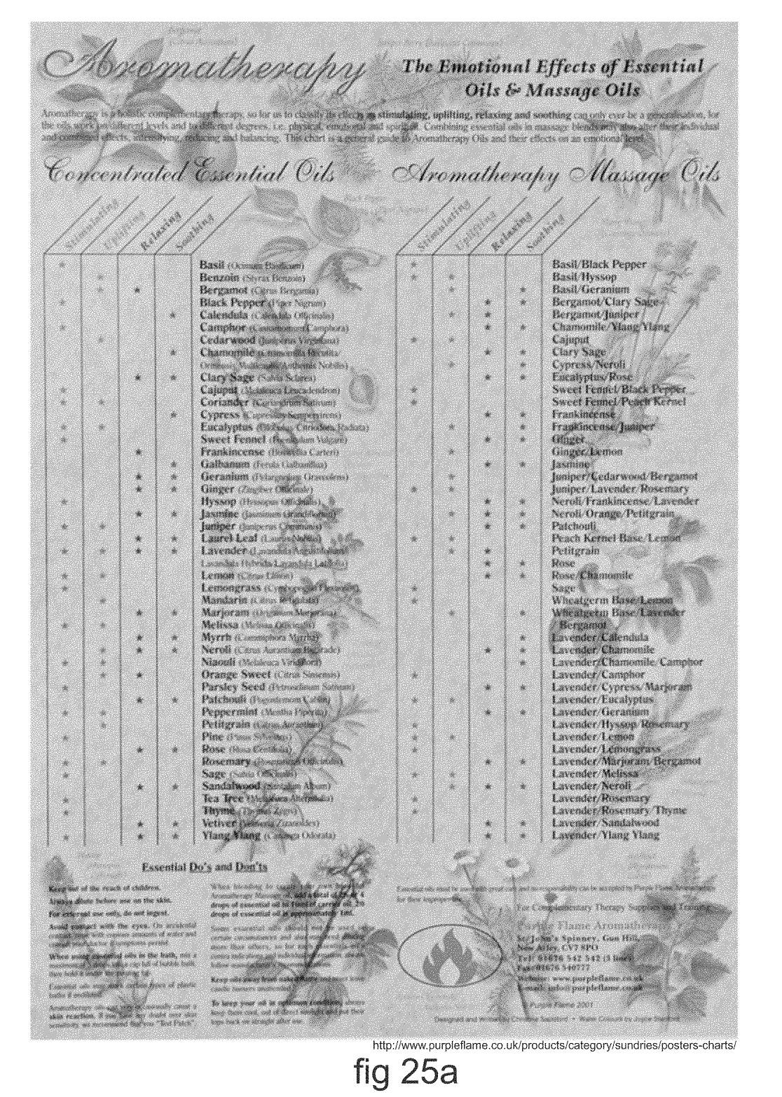

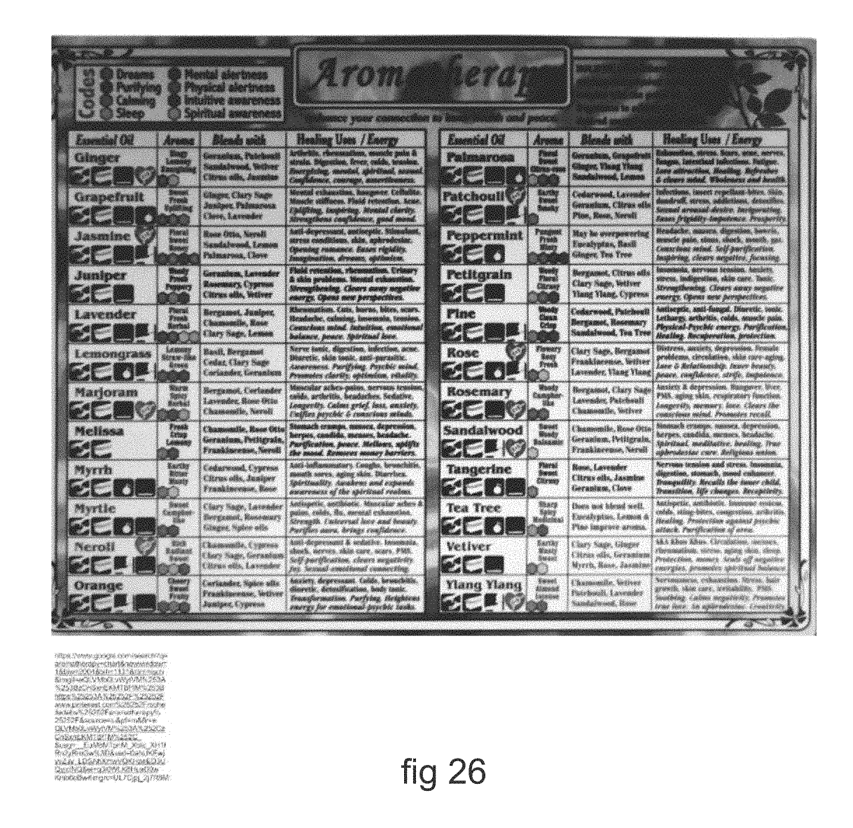

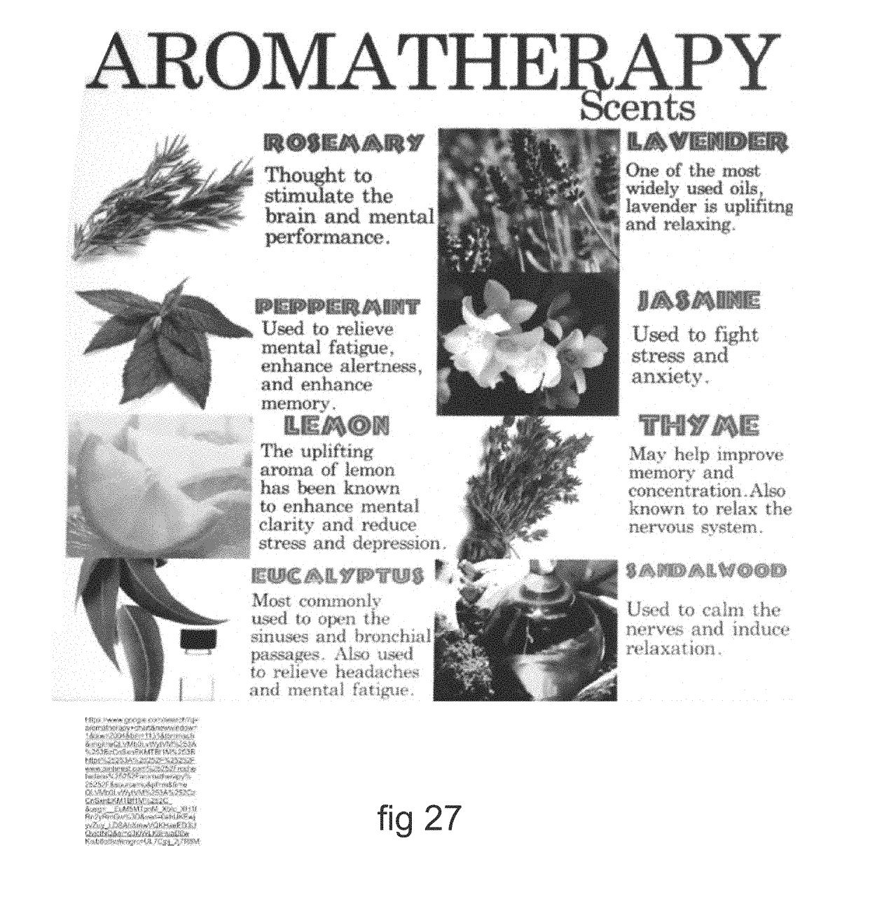

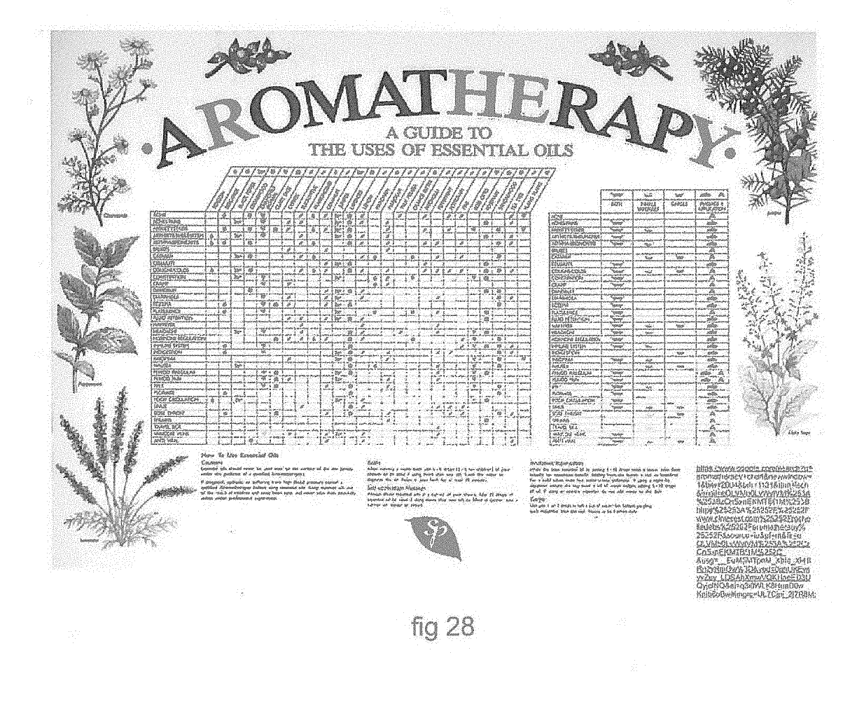

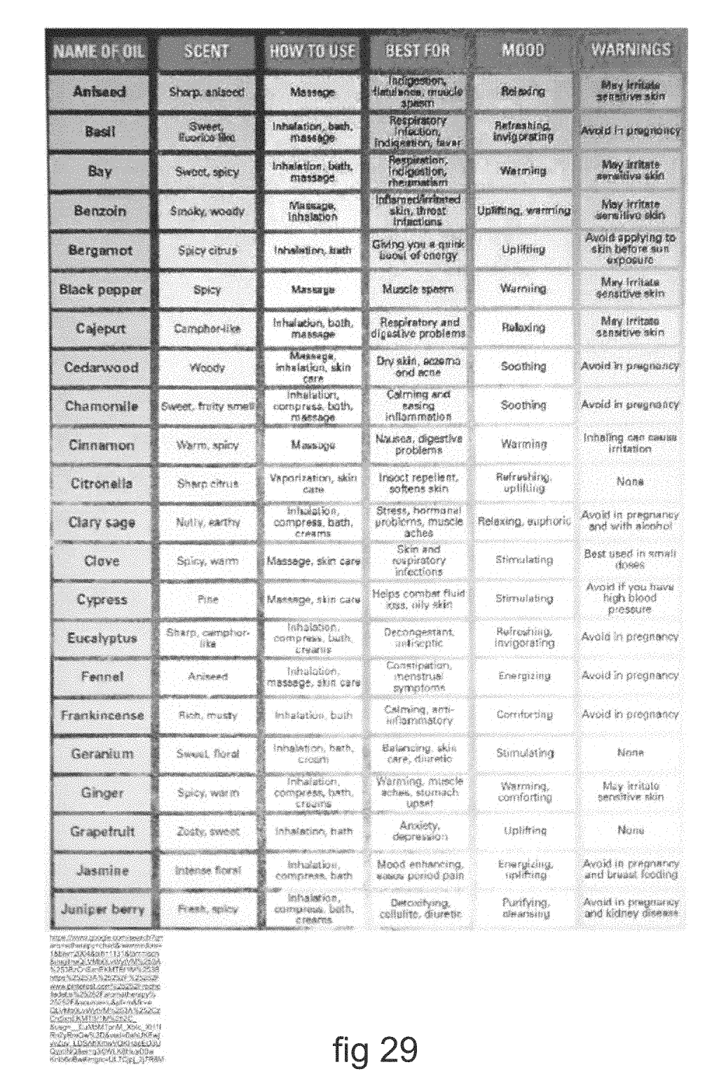

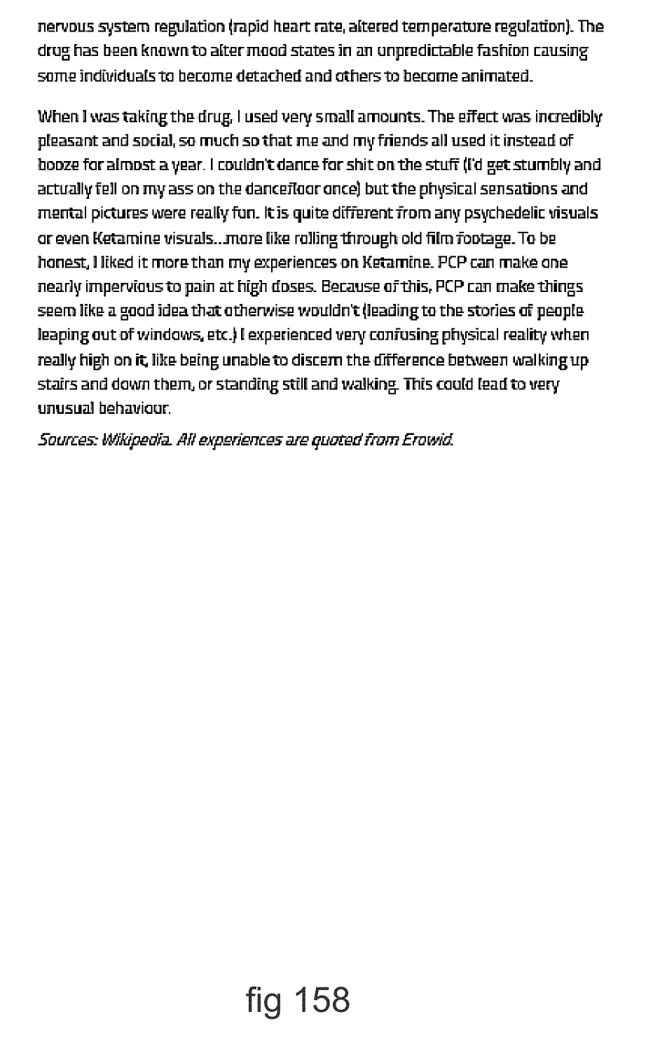

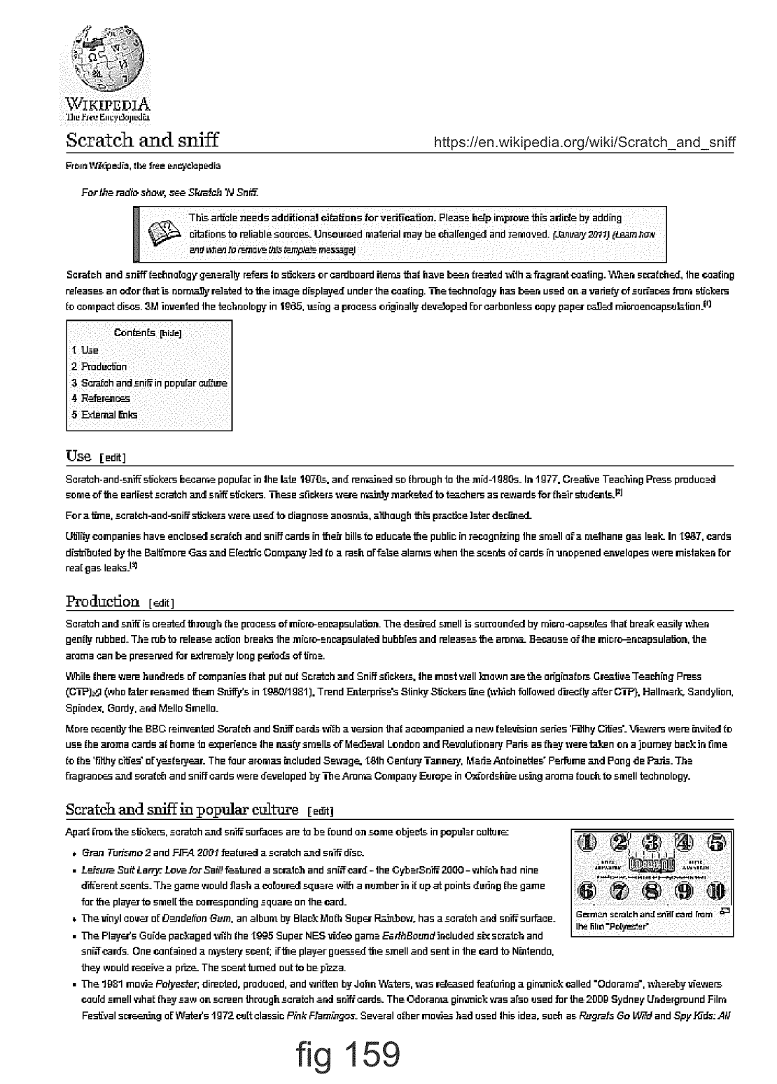

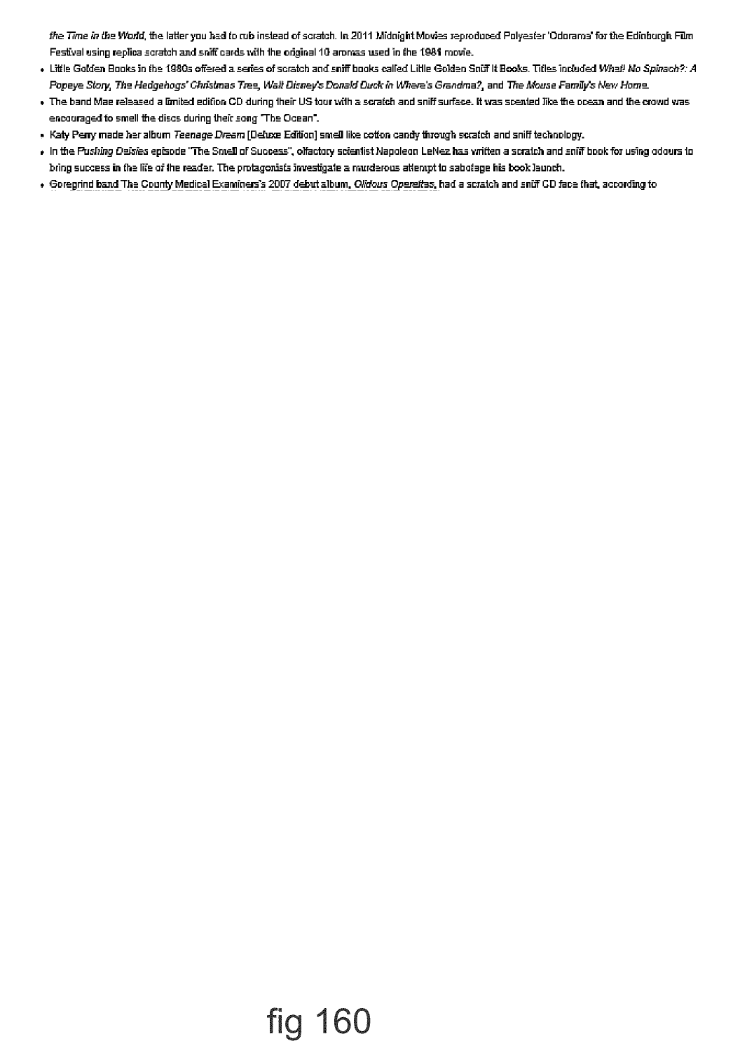

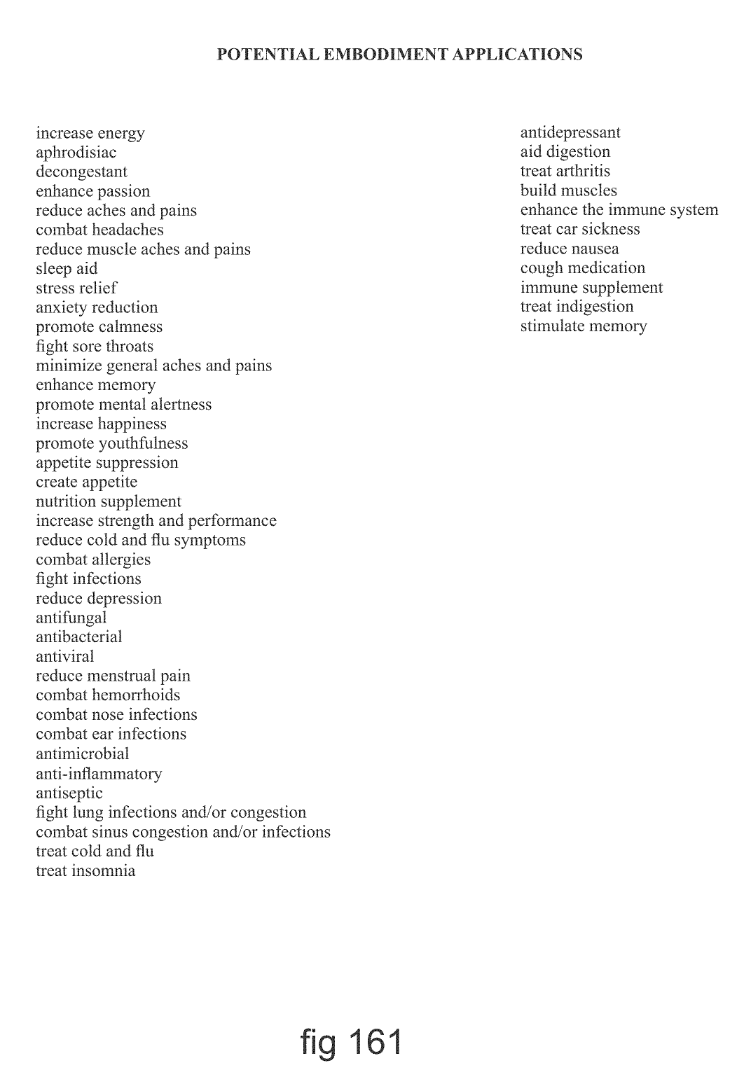

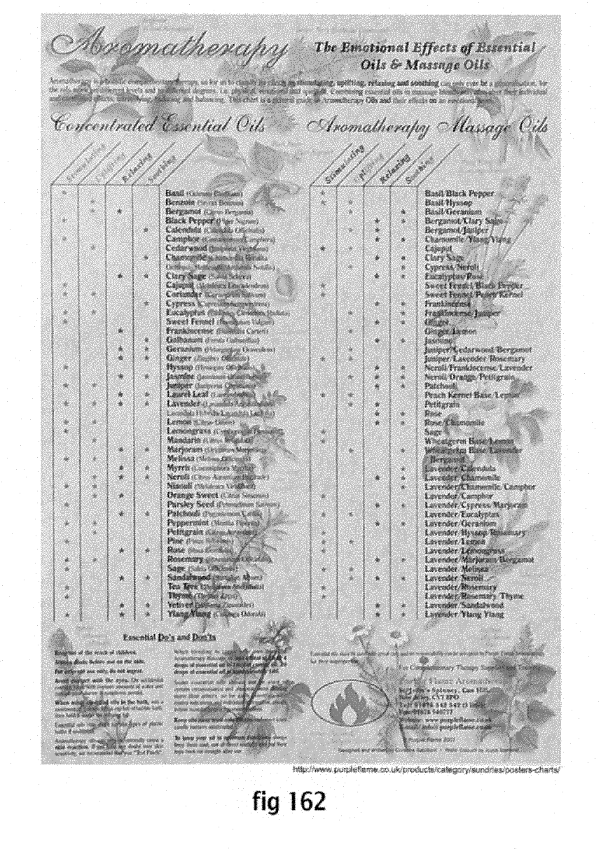

FIGS. 20 through 29, and FIGS. 56 through 158 show a non-limiting and non-exhaustive compilation of active ingredients which might be considered for use in embodiments herein.

FIG. 159 give details of scratch and sniff technology.

FIG. 160 give details of scratch and sniff technology.

FIG. 161 shows non-limiting and non-exhaustive examples of how embodiments of the subject application might be utilized.

FIG. 162 is background corresponding to aroma therapy.

### DETAILED DESCRIPTION OF EMBODIMENTS

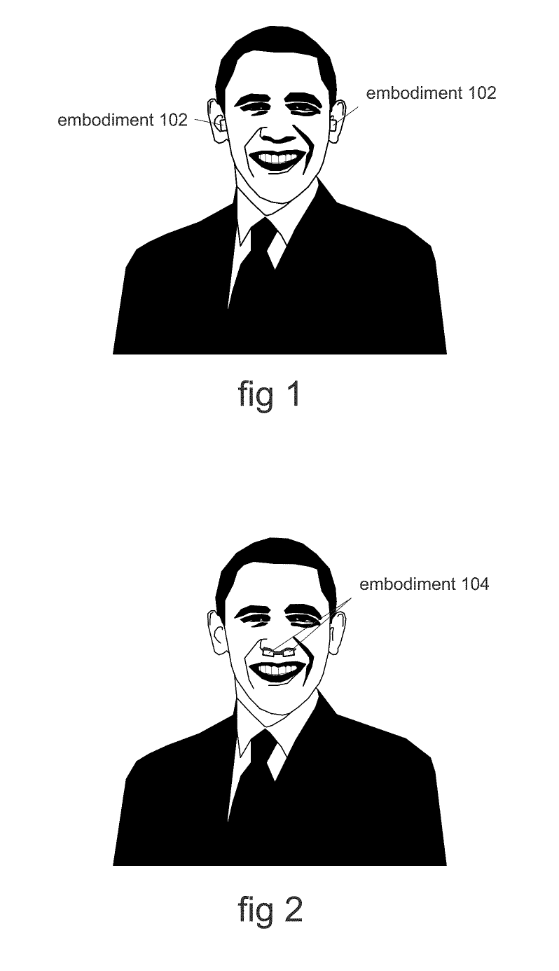

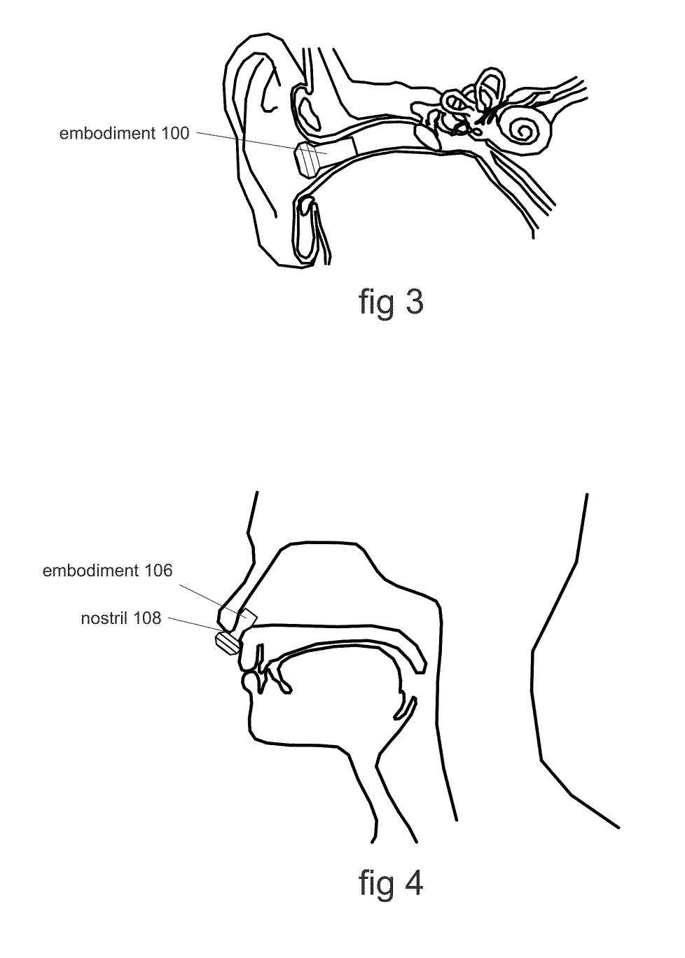

Referring to FIGS. 1 through 4, as both non-limiting and non-exhaustive examples, embodiments 100, 102, 104, and 106 are shown inserted into human ears (embodiments 102 and 100 in FIGS. 1 and 3 respectively), and into human nostrils (embodiments 104 and 106 in FIGS. 2 and 4 respectively).

As further non-limiting and non-exhaustive examples, such embodiments might also be inserted into human mouths, anuses, and vaginas.

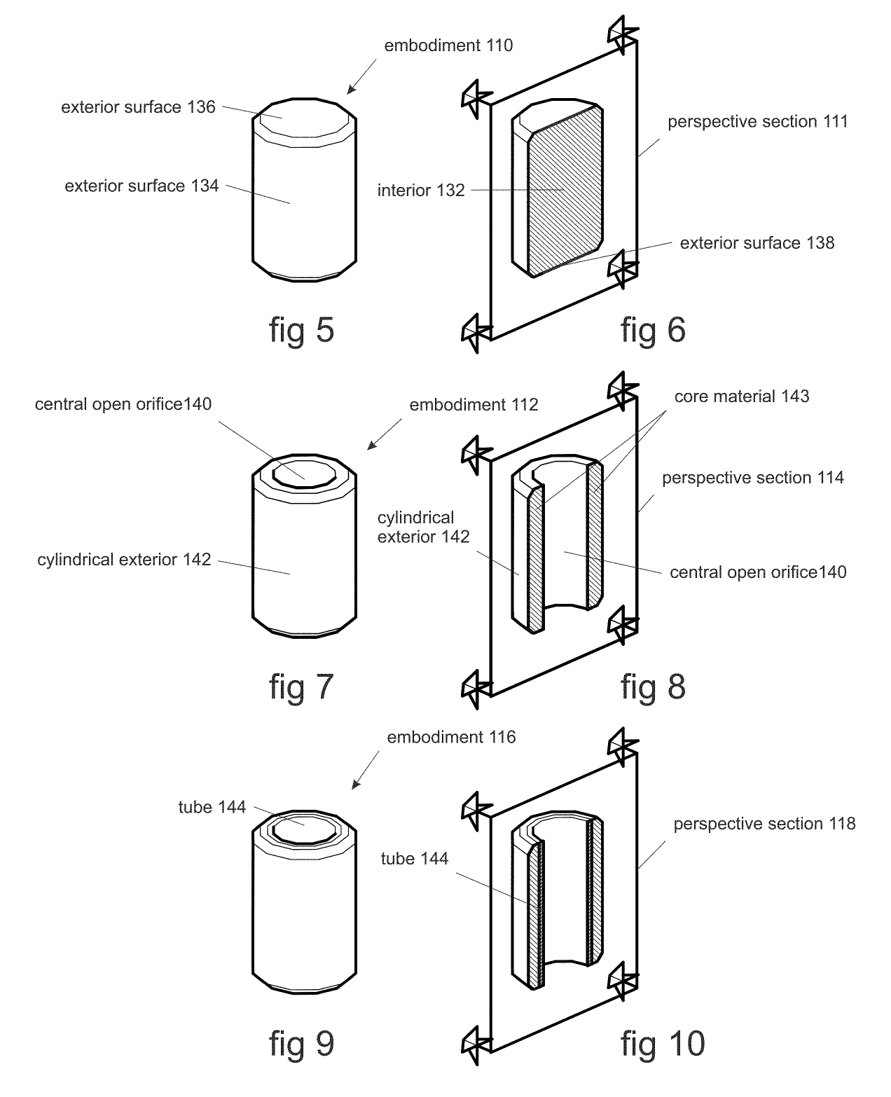

Such embodiments (100, 102, 104, and 106) might be of suitable construction, including, but not limited to, construction shown in FIGS. 5 through 16.

FIG. 5 shows embodiment 110: Embodiment 110 is a generally cylindrical block

constructed from a formable material, such as, as non-limiting and non-exhaustive examples, a formable plastic foam, or a formable fibrous material. This may be rigid, semirigid, and/or resilient.

It also may be permeable to active ingredients. Again, as a non-limiting and non-exhaustive examples it may be an open cell foam or a formed fibrous material, such as cellulose composites used as cushioning in commercial packaging, and in fast food cupholders.

Depending on construction, gas permeability might allow air and/or sound to pass through embodiment 110.

As a non-limiting and non-exhaustive example, reticulated foam, such as used for speaker grill covers, microphone covers, and earbud caps, might be used in embodiment 110, which in turn might allow embodiment 110 to be inserted into ears, while allowing sound to pass through; and to be inserted into nostrils, while allowing breathing through the nose to take place.

Embodiment 110 might have membranes, which are non-permeable, semi permeable, or permeable to active ingredients, covering one or more exterior surfaces 134, 136 and 138. Such membranes might allow more precise control of the dispersion of active ingredients.

Embodiment 110 might be constructed of resilient material. Resiliency, in any of the embodiments contained herein, might ease insertion effort, aid in transmission of active ingredients, and also help promote a secure and comfortable fit.

Active ingredients delivered by embodiment 110, might reside in interior 132, and/or might reside on one or more exterior surfaces 134 136 and 138.

In general, the term active ingredient when used herein, means any substance which alters biological functions or states, be they physical or mental. As non-limiting and non-exhaustive examples, this may be inclusive of, but not necessarily limited to, volatile and nonvolatile liquids, volatile and nonvolatile semi liquids, such as creams, syrups, or pastes; volatile and nonvolatile solids, inclusive of those which effervesce in air and/or body heat; and/or may be dissolved by fluids, including, but not limited to, body fluids, or of any other suitable form. Active ingredients, as used herein, may also include fluids in general, be they liquid and/or gaseous; as well as semi liquids, semi solids and/or solids and/or other matter states.

FIG. 7 shows embodiment 112: Embodiment 112 is similar in construction to embodiment 110, except for having open orifice 140 disposed generally centrally within generally cylindrical exterior 142.

Embodiment 112 may share construction details and active ingredient potentials with embodiment 110.

Central open orifice 140 allows sound and/or air to pass through. Because of this, as a non-limiting and non-exhaustive example, when used in nose and mouth; embodiment 112 may allow for breathing. And when used in years, embodiment 112 may allow for hearing.

Embodiment 112 may be constructed from materials such as slow rebound foam, or resilient close cell foam, or other suitable materials, and still allow hearing and/or breathing. Slow rebound foam is used in memory foam mattresses and disposable sound blocking earplugs, such as are given out on airlines.

Embodiment 112, may deliver plural active ingredients. As a non-limiting and non-exhaustive example, when intended for insertion into nostrils, cylindrical exterior 142 might be coated with a non-permeable flexible membrane coated on its exterior with a first osmosis delivered active ingredient, while core material 143 might contain a second active ingredient which is volatilized by body heat and is delivered by being breathed in through central open orifice 140.

Central open orifice 140 may or may not be coated with a membrane which may or may not be permeable or non-permeable to active ingredients. As a non-limiting and non-exhaustive example, such a membrane might be used to control the dissemination of active ingredients.

FIGS. 9 and 10 show embodiment 116: Embodiment 116 is similar in construction to embodiment 112, with the addition of flexible or rigid tube 144, disposed generally at its center. Tube 144 may be permeable or non-permeable to active ingredients.

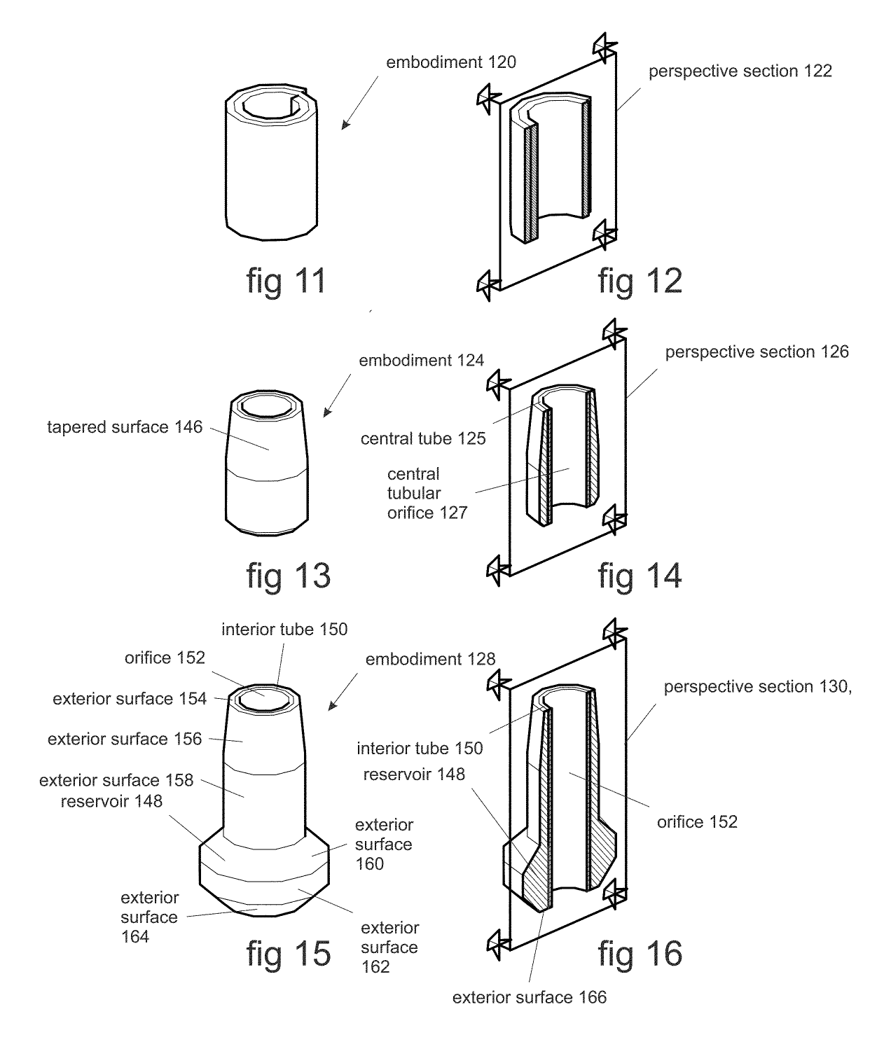

FIGS. 11 and 12 show embodiment 120: Embodiment 120 is essentially a generally rectangular piece of material which can hold active ingredients, and which is rolled into a tubular form.

As a non-limiting and non-exhaustive example, embodiment 120 can be constructed from felt as a substrate.

FIGS. 13 and 14 show embodiment 124: Embodiment 124 shares construction details with embodiment 116, except that embodiment 124 has tapered surface 146, which may aid in the easy insertion of embodiment 124 into body cavities. Embodiment 124 may have variants which are analogous in construction to embodiments 112 and 110, with no central tube 125, and/or no central tubular orifice 127.

FIGS. 15 and 16 show embodiment 128: Embodiment 128 shares construction details with embodiment 124, with the addition of reservoir 148 which, during use, is disposed exterior to the inserted body cavity (like shown in FIGS. 3 and 4).

Like embodiment 124, embodiment 128 can have variants which are analogous in construction to embodiments 112 and 110, with no central tube 125, and/or no central tubular orifice 127.

Interior tube 150 may be flexible or rigid, and may be permeable or nonpermeable to active ingredients.

Exterior surfaces 154, 156, 158, 160, 162, 164 and 166 may, each, any, or all; be covered with membranes which are either permeable or non-permeable to active ingredients, and/or which may hold active ingredients.

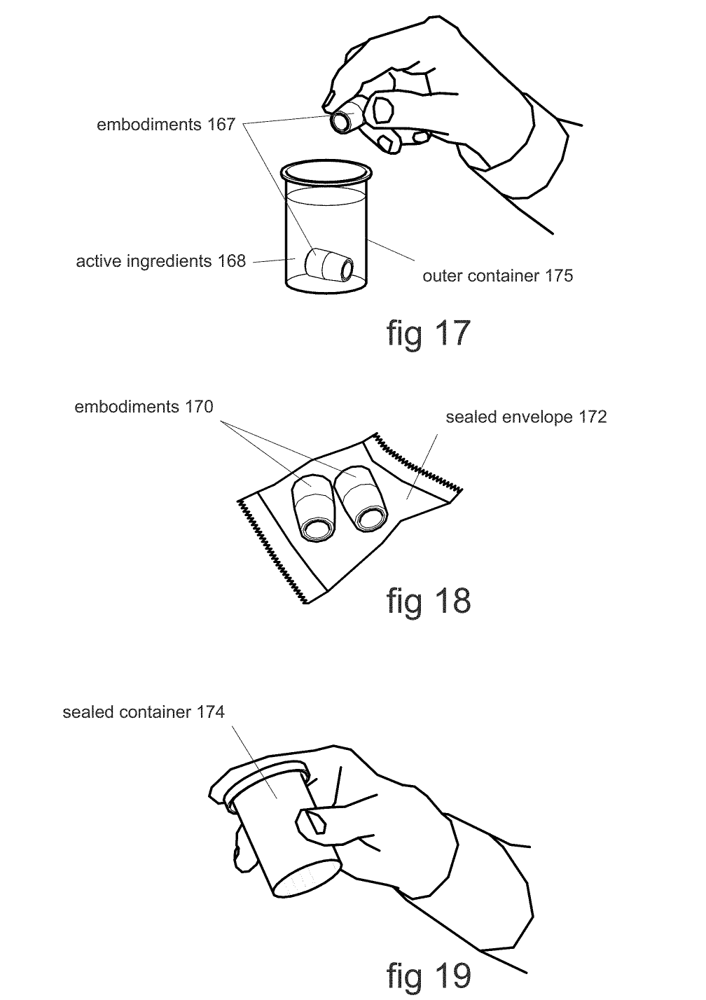

FIG. 17 shows preparation and/or packaging of embodiments 167, which may be any of the embodiments already described herein, by dropping said embodiments 167 into active ingredients 168, which may be liquid, semi liquid, powders, or other physical states.

FIG. 18 shows embodiments 170 inside of sealed envelope 172. Such packaging, as a non-exhaustive and non-limiting example, could be non-permeable to active ingredients. Embodiments 170 could be preloaded with active ingredients, or might be placed inside of envelope 172 along with active ingredients.

Likewise, as a non-limiting and non-exhaustive example, sealed envelope 172 could be re-sealable utilizing a zip closure. This would allow active ingredients to be added to envelope 172 to prepare embodiments 170 for use. This might also allow a way for embodiments 170 to be packaged between uses, if multiple uses were specified.

Embodiments 170 could be any of the embodiments contained herein, including, but not necessarily limited to: embodiments 110, 112, 116, 120, 124, and 128.

FIG. 19 shows re-sealable sealed container 174: Sealed Container 174 is similar in function to, and may be used as a replacement for, sealed envelope 172, or outer container 175 (FIG. 17).

FIGS. 20 through 29 are documents which are generally related to aromatherapy.

As shown in the documents shown in FIGS. 20 through 29, practitioners of aromatherapy claim specific aromatherapy active ingredients may predictably alter physical and/or mental states, and/or promote healing, and/or have other effects.

Many other biological altering substances may also benefit from use of embodiments herein.

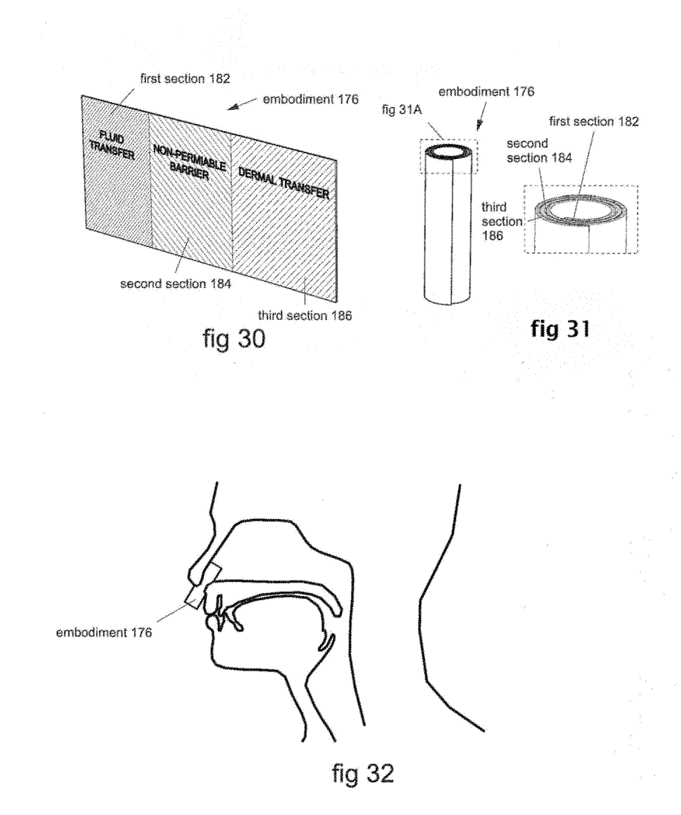

FIGS. 30 and 31 show embodiment 176: Referring to FIG. 30, embodiment 176 is comprised of a generally sheet-like rectangular strip, which is configured to be rolled up, as shown in figure. This rectangular strip includes three discrete sections.

First section 182 (nearest the rolled up center—FIGS. 31 and 31 A) includes substrate which may disperse fluid and/or other active ingredients, including, but not limited to gases, vapors, colloidal suspensions, as well as other fluids, as well as other compositions.

These may be activated by exposure to ambient atmosphere, or by body heat, or by breathing, or by other means.

Second section 184 of embodiment 176 is comprised of a barrier which is non-permeable to active ingredients within embodiment 176.

Third section 186 (exterior and farthest from the center) includes substrate holding active ingredients which may be delivered through contact with outer body surfaces, such as, by way of non-limiting and non-exhaustive examples, inner nose surfaces, or surfaces of ear canals.

As shown in FIG. 31, embodiment 176 is configured to be rolled up, with first section 182 on its interior. Rolling embodiment 176 makes it easy to adapt embodiment 176 to various nostril sizes, simply by rolling it tighter or looser.

Substrate for first section 182 and third section 186 may be fabricated from any suitable material. As non-limiting and non-exhaustive examples, it may made from fabric, felt, pliable foam, resilient or slow rebound foam, open or closed cell foam, self-skinning foam, combinations of any of the foregoing, or any other suitable material.

Non-permeable barrier, section 184, as non-exhaustive and non-limiting examples, could be constructed from aluminum foil, Mylar plastic, or other suitable material.

Embodiment 176 may be particularly appropriate for insertion into ears, nose, or mouth.

In use, as non-limiting and non-exhaustive examples, 2 embodiment 176s might be shipped flat in a sealed, non-permeable to active ingredients, plastic bag. The user would simply open the bag, roll 2 embodiment 176s to the desired diameter, and insert them into their nostrils. After use, the user may simply dispose of the embodiment 176s, or save them for future use.

As a non-limiting and non-exhaustive example, 2 embodiment 176s might be rolled, and sealed inside a non-permeable plastic zipper bag before leaving the factory, leaving the user only to open the bag and insert the embodiment 176s into their ears. After use, the two embodiment 176s might be disposed of or might be stored in the plastics zipper bag.

Embodiment 176 might also be inserted into ear canals, as shown in FIGS. 1 and 3.

FIGS. 34 through 37 show embodiment 178, which, like embodiment 176, is configured to transfer active ingredients simultaneously through inhalation and exterior body surface contact.

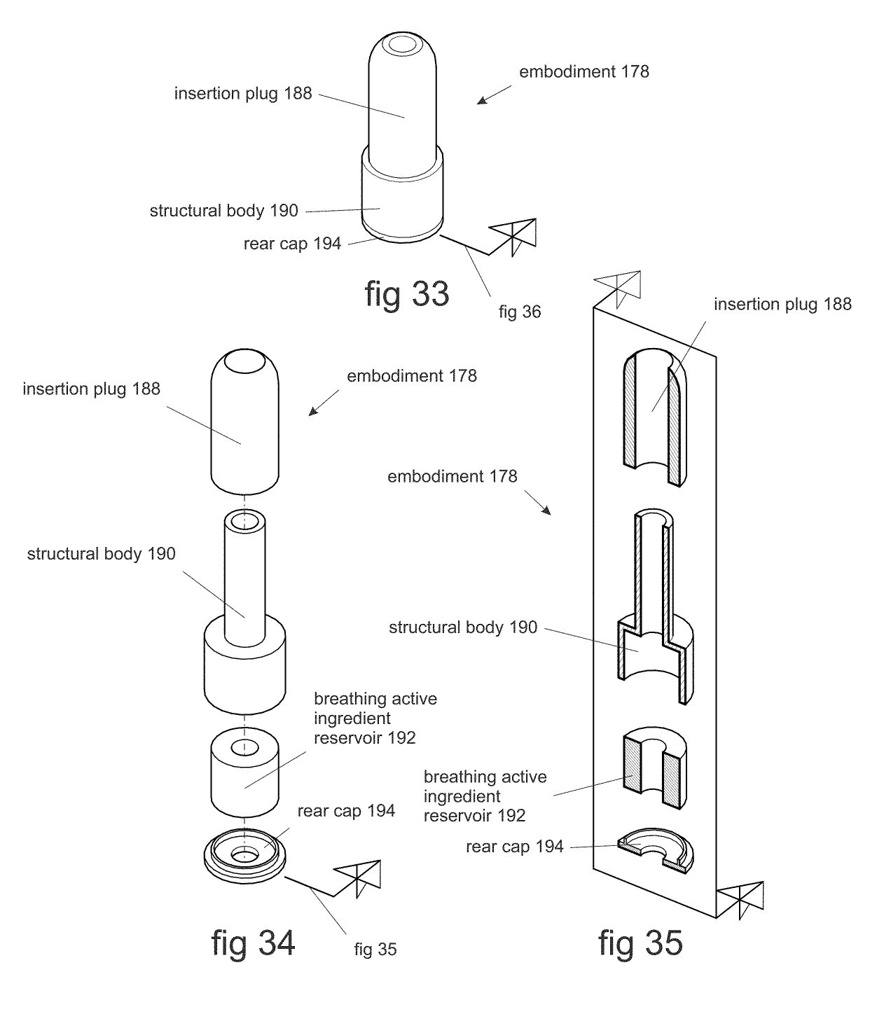

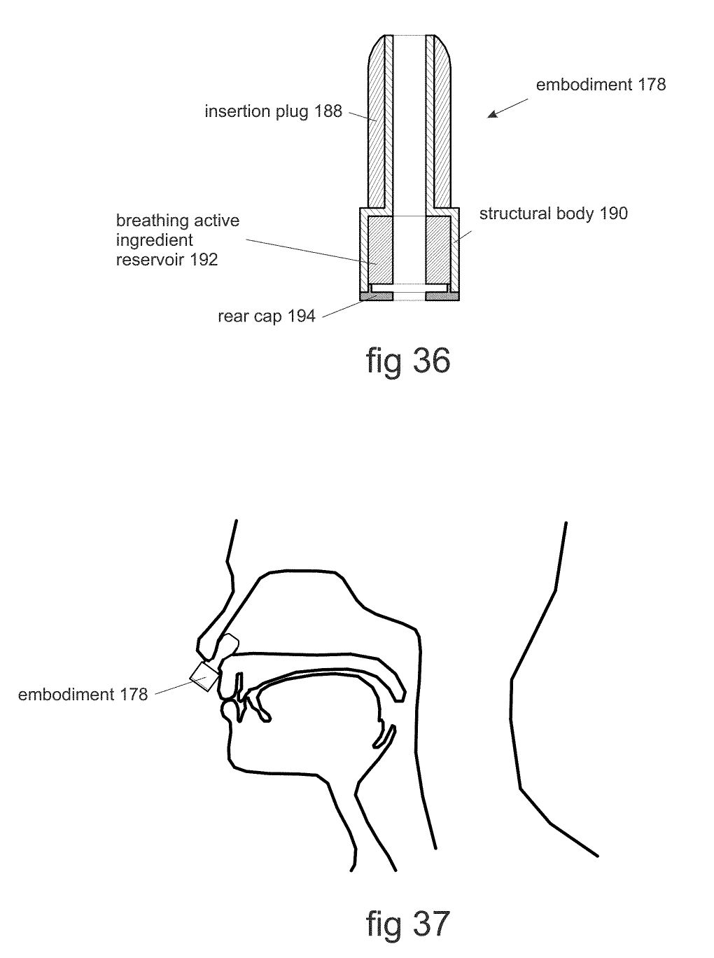

As shown best in FIGS. 33 through 36, embodiment 178 is essentially comprised of four pieces: insertion plug 188, structural body 190, breathing active ingredient reservoir 192, and rear cap 194. The four pieces are simply snapped together as shown best in FIGS. 33 and 36.

As a non-limiting and non-exhaustive example insertion plug 188 might be fabricated from a self-skinning slow rebound foam, such as used in disposable earplugs the airlines give out.

Insertion plug 188 would contain the active ingredients to be delivered through external body surface contact.

Structural body 190, and rear cap 194, as non-limiting and non-exhaustive examples, might be fabricated by injection molding plastic, such as polypropylene, or ABS.

Also as a non-limiting and non-exhaustive example, breathing active ingredient reservoir 192, might be fabricated from resilient or rigid plastic foam, such as polyurethane foam. Again its active ingredients may be activated by body heat, breath, exposure to atmosphere, or by other suitable means.

In use, as non-limiting and non-exhaustive examples, 2 embodiment 178s might be vacuum packed in a plastic envelope. The user would simply open the envelope, and insert the 2 embodiment 178s into their nostrils. After use, again, the 2 embodiment 178s might simply be disposed of, or stored for future use.

As a non-limiting and non-exhaustive example, embodiment 178 might also be inserted into ear canals, as shown in FIGS. 1 and 3.

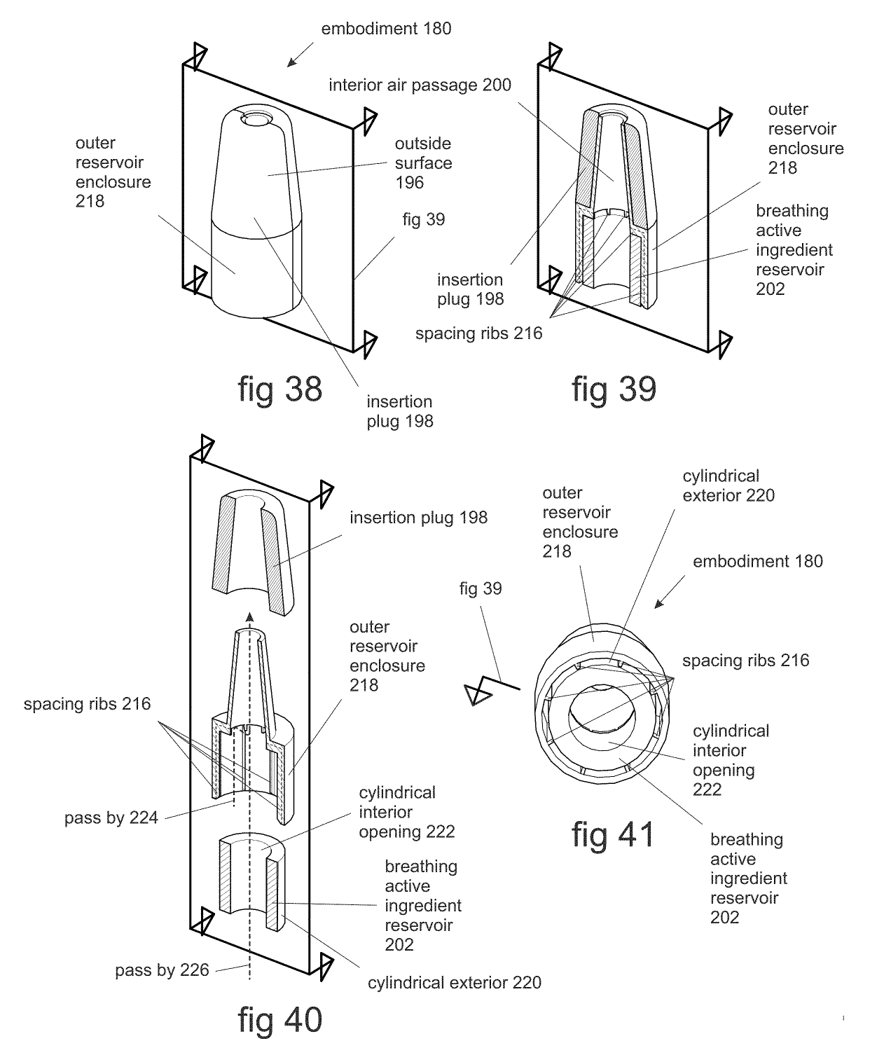

FIGS. 38 through 41 show embodiment 180. Embodiment 180 is a variant of embodiment 178, and may share manufacturing and use features. Embodiment 180 differs from embodiment 178, by tapering outside surface 196, of insertion plug 198, making it both easier to insert, and more adaptable to various nostril and/or ear canal sizes.

Embodiment 180 also differs from embodiment 178 by tapering interior air passage 200. This may allow breathing air or sound to pass through it more freely.

Finally embodiment 180 differs from embodiment 178 by allowing breathing air to pass by all sides of breathing active ingredient reservoir 202. This may facilitate transference of active ingredients within breathing active ingredient reservoir 202 into air passing through it.

More specifically, spacing ribs 216, projecting inward and downward from outer reservoir enclosure 218, distance breathing active ingredient reservoir 202 from inner walls of outer reservoir enclosure 218. This allows incoming air to both pass by 224 cylindrical exterior 220 of breathing active ingredient reservoir 202, as well as to pass by 226 cylindrical interior opening 222.

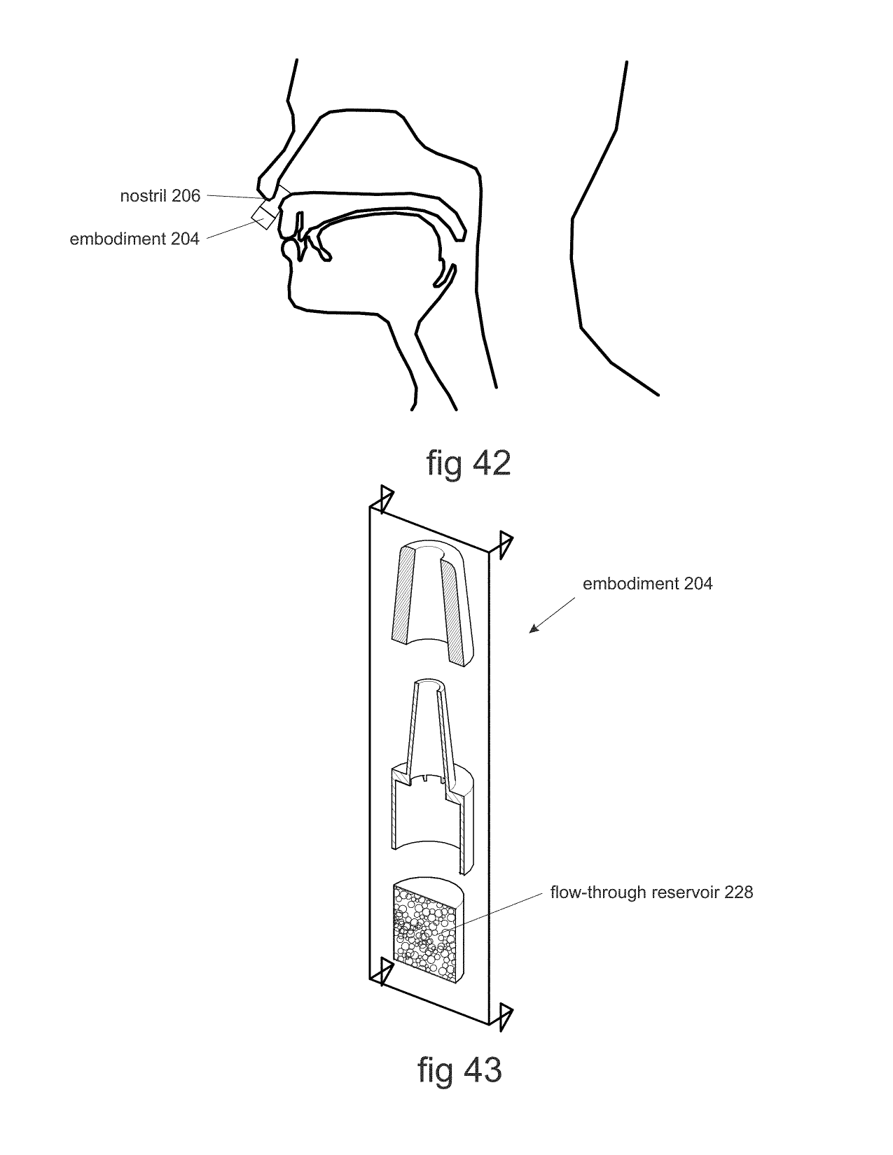

FIGS. 42 and 43 show embodiment 204. Embodiment 204 is a variant of embodiment 180 which substitutes flow-through reservoir 228, holding active breathable ingredients, for breathing active ingredient reservoir 202. Flow-through reservoir 228 may be constructed of any suitable material which can both retain active ingredients, and allow air to pass by them. As non-limiting and non-exhaustive examples, it might be constructed from reticulated foam, or nonwoven polyester, or cotton, or other suitable material.

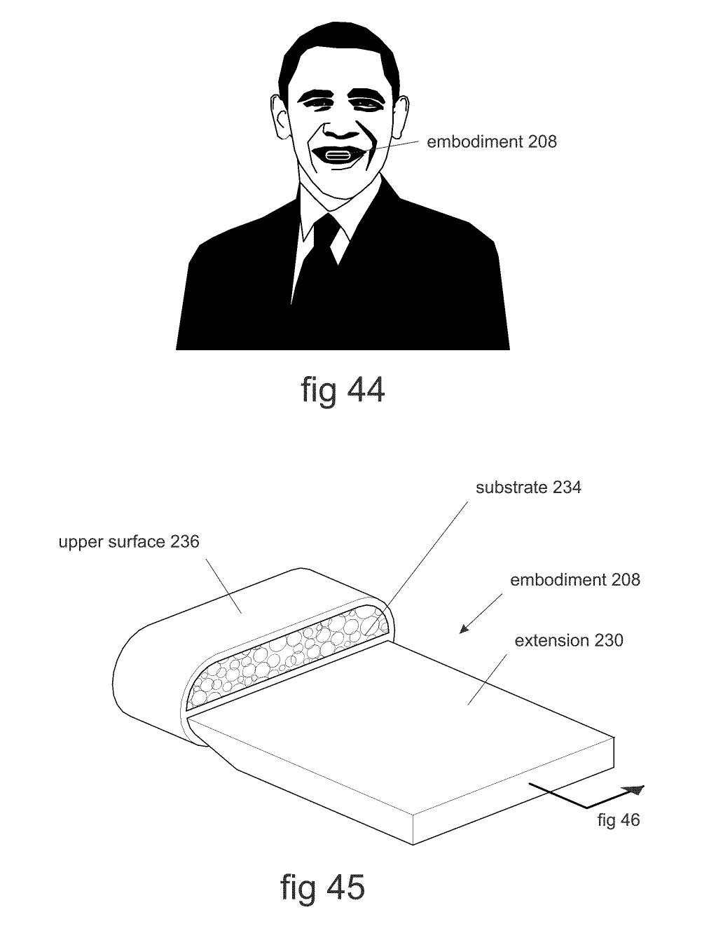

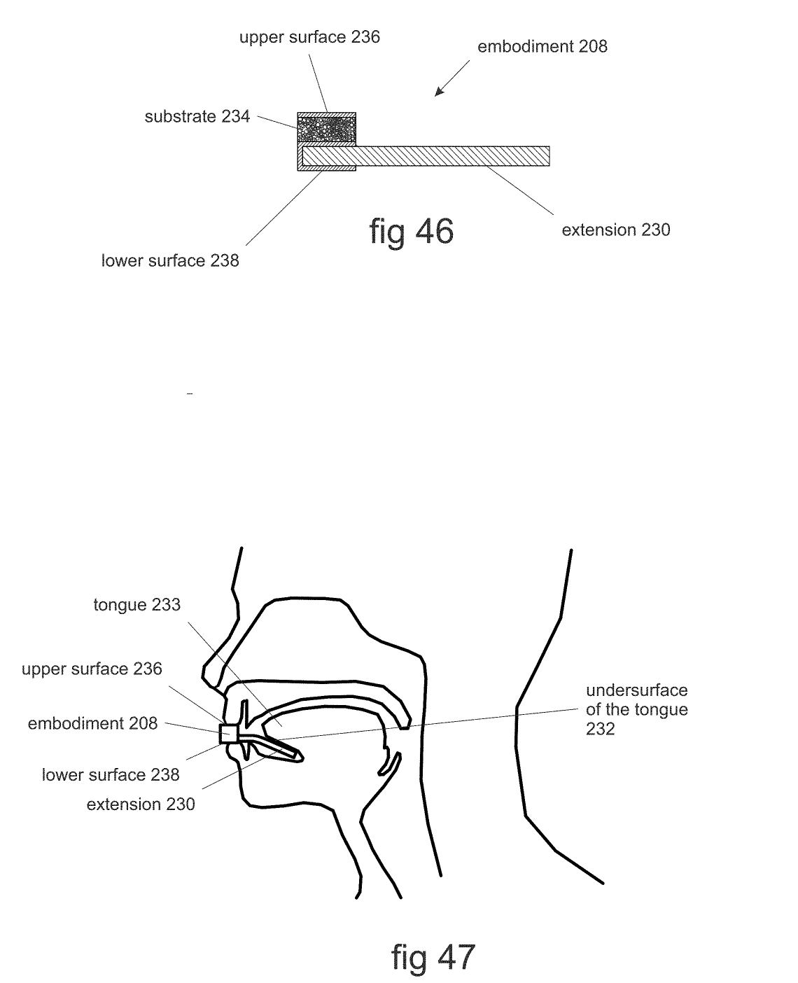

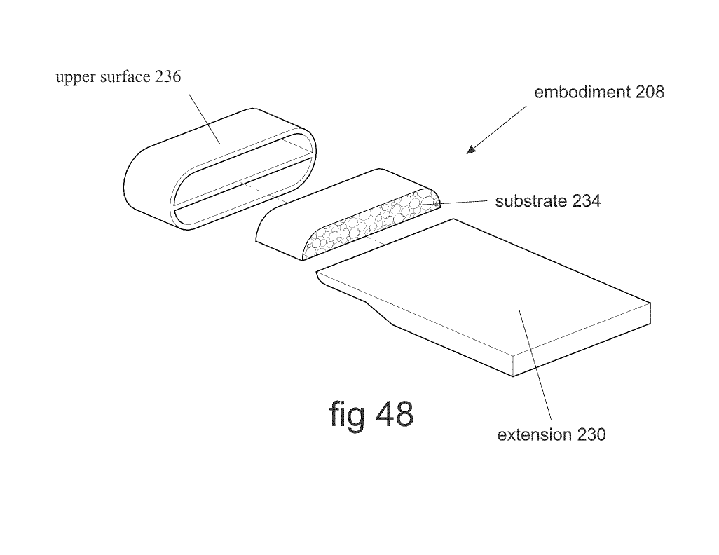

FIGS. 44 through 48 show embodiment 208. Embodiment 208, as best shown in FIGS. 44 and 47, is placed in the mouth, with extension 230 projecting inward to the undersurface of the tongue 232 (FIG. 47). This sublingual positioning allows transference of active ingredients carried by extension 230 directly to sublingual surfaces, which are efficient in passing active ingredients directly into the bloodstream.

Embodiment 208, as a non-limiting and non-exhaustive example, like other embodiments herein, may include specific instructions on their use

Substrate 234 is an air transmissive material configured to hold active ingredients and transfer active ingredients to air passing through substrate 234.

In use, embodiment 208 is placed in the mouth, with lips sealing against upper surface 236 and lower surface 238; and extension 230 projecting into the mouth and contacting undersurface 232 of tongue 233 (FIGS. 44 and 47 in particular).

Depending on the desired outcome, active ingredients which are effective when inhaled, are contained within substrate 234, and active ingredients which may be effective using sublingual transfer are contained within extension 230.

As with all embodiments herein which deliver active ingredients with two or more transfer means (in this case, through inhalation and sublingual transfer), active ingredients contained within substrate 234 can at least be: complementary to, in opposition to, or non-associated with, active ingredients contained within extension 230.

As a non-limiting and non-exhaustive example, essential oils of juniper, which have an invigorating effect, might be contained within substrate 234; and an amphetamine, which also has a stimulating effect, might be contained in extension 230. Each active ingredient reinforcing the energy imparting effects of the other. This makes the two active ingredients complementary to, or cooperative with one another.

As another non-limiting and non-exhaustive example, essential oils of Juniper might be contained within substrate 234, and a barbiturate, which has a calming effect, might be contained in extension 230, providing both energy and relaxation. This constitutes two ingredients providing opposing effects.

As yet one more non-limiting and non-exhaustive example, essential oils of Juniper might be contained within substrate 234, providing a stimulating effect; while Duloxetine HCL (CYMBALTA), which is an anti-depressant, might be contained in extension 230. The first active ingredient (Juniper) in this case providing a stimulating effect, which is non-associated with the antidepressant effect of the second active ingredient (Duloxetine HCL).

It should be noted that active ingredients listed herein, as well as any other suitable active ingredients, may be currently illegal in some localities, or legal only with specific restrictions, such as, as non-limiting and non-exhaustive examples, by prescription only, or for medical or research applications only. Teachings herein are intended for use only where, when, and under conditions that their application complies with all relevant laws.

It should also be noted that relevant laws to the above are currently changing, and may be changed in the future; and that no one has ways of predicting such changes. Certain embodiments which may be reasonably derived from teachings herein, whereas not lawful today, may become lawful in the future, including during statutory periods which may be covered by derivatives of this application document.

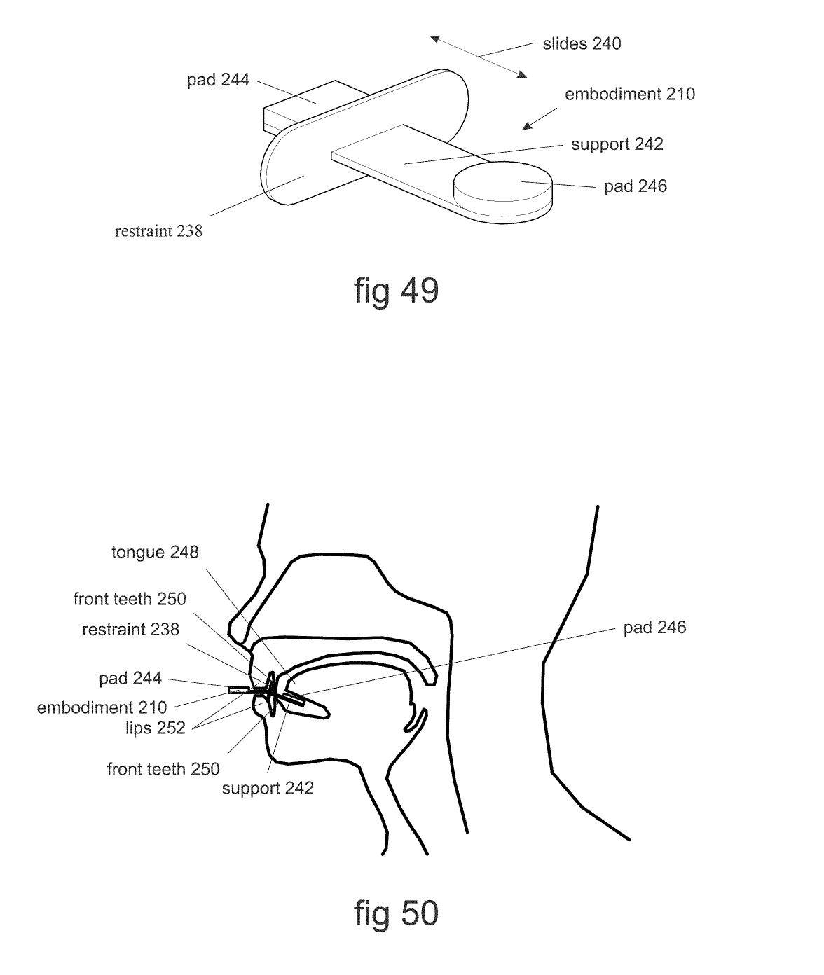

FIGS. 49 and 50 show embodiment 210: Embodiment 210 is comprised of: restraint 238, which is mounted on, and slides 240 along, support 242. Mounted to one end of support 242 is pad 244, which contains active ingredients configured to be inhaled through the nose.

Mounted to the opposite end of support 242, is pad 246, which delivers active ingredients sublingually.

In use, restraint 238 is adjusted along support 242 so that embodiment 210 fits comfortably into the mouth, with pad 246 resting on the underside of tongue 248. As shown best in FIG. 50, restraint 238 is fitted to rest between the front of the front teeth 250, and the back of the lips 252.

Pads 244 and 246 may each or both be configured to have outer coverings which control active ingredient distribution and transfer properties. Such outer coverings may regulate distribution rates, and/or they may facilitate active ingredient transference, as a non-limiting and non-exhaustive example, using texture and/or form.

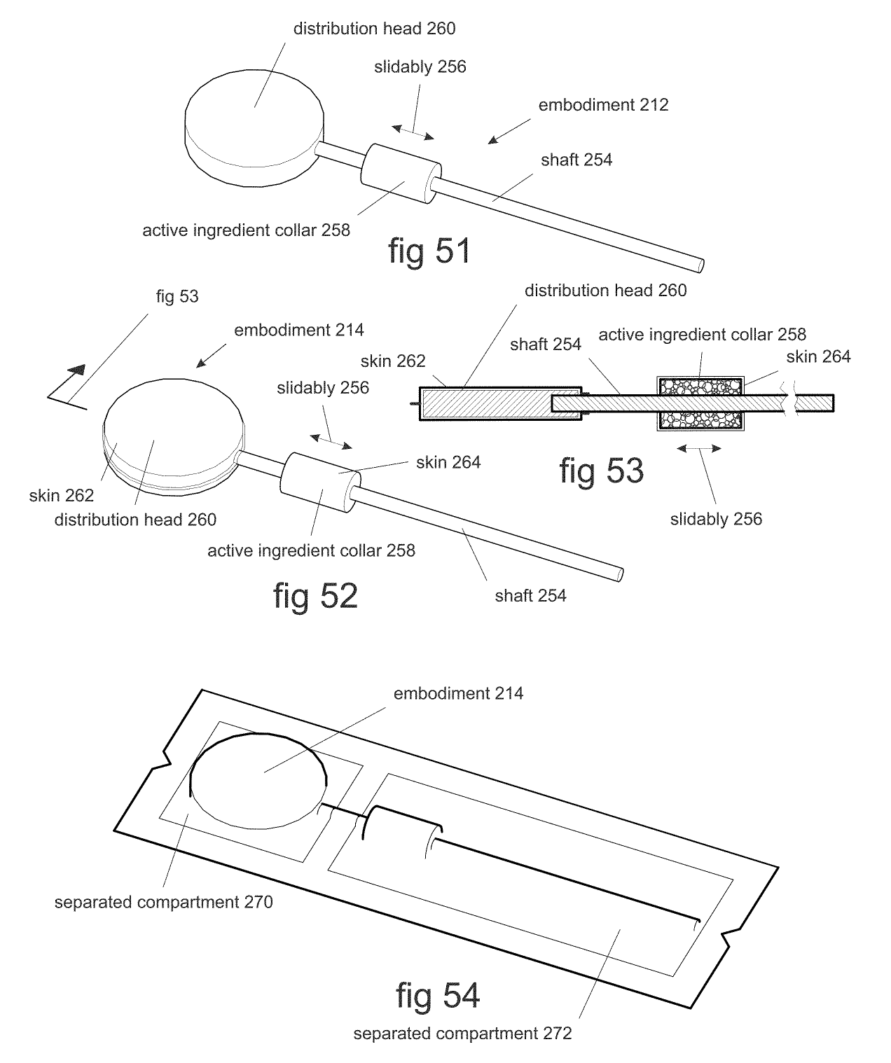

FIGS. 51 through 55 show embodiments 212 and 214: Embodiment 212 is comprised of shaft 254, which slidably 256 and removably mounts active ingredient collar 258. Mounted on one end of shaft 254, is distribution head 260.

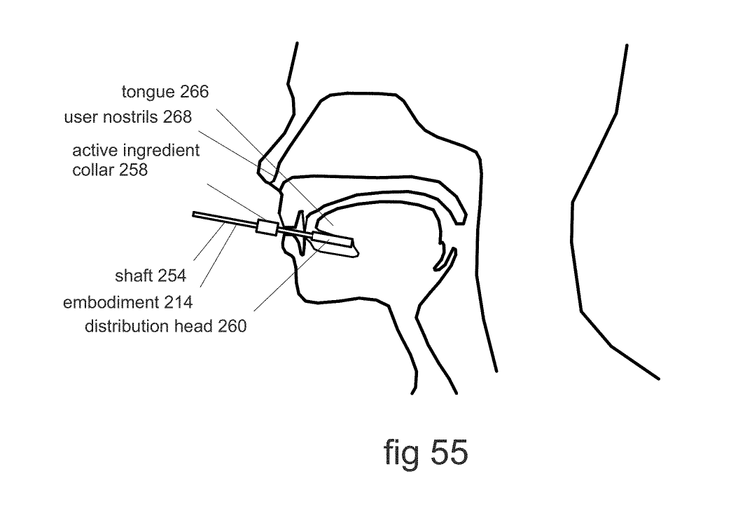

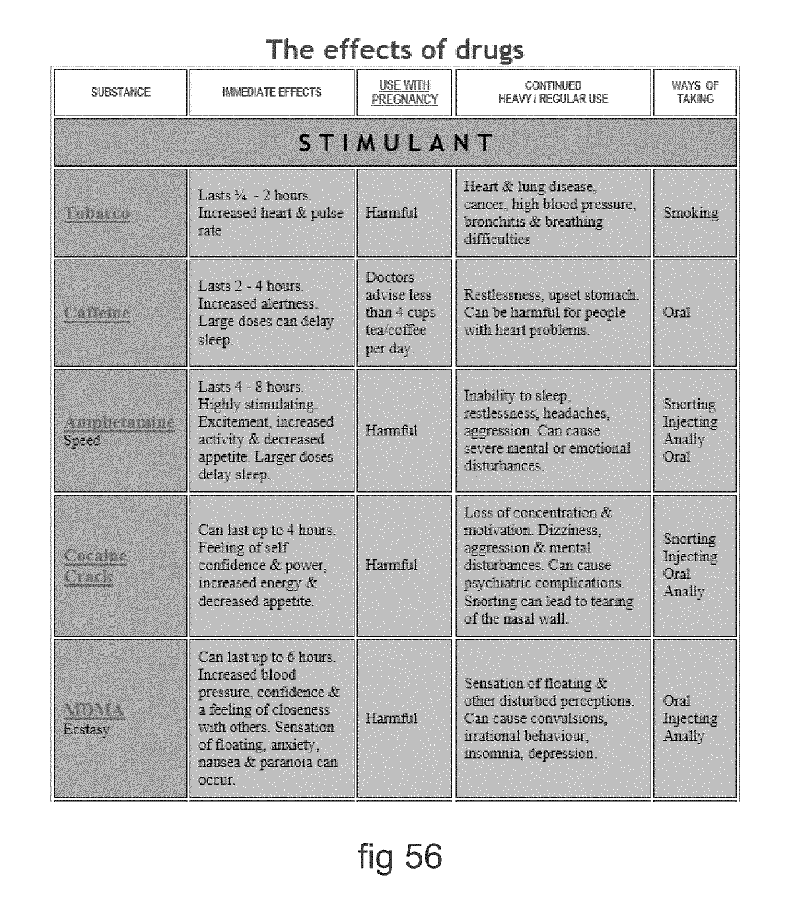

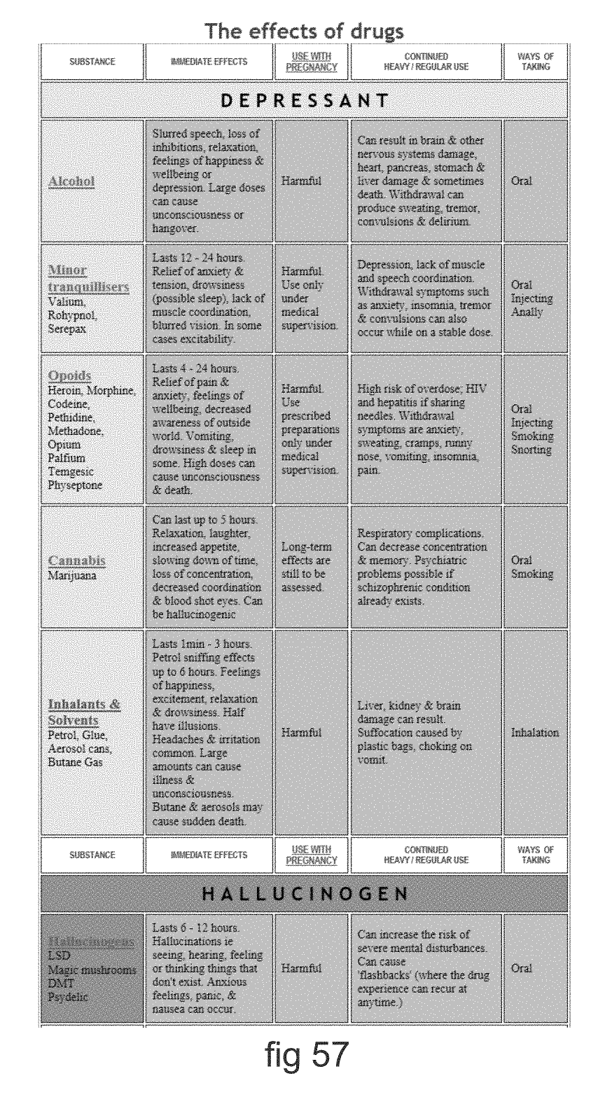

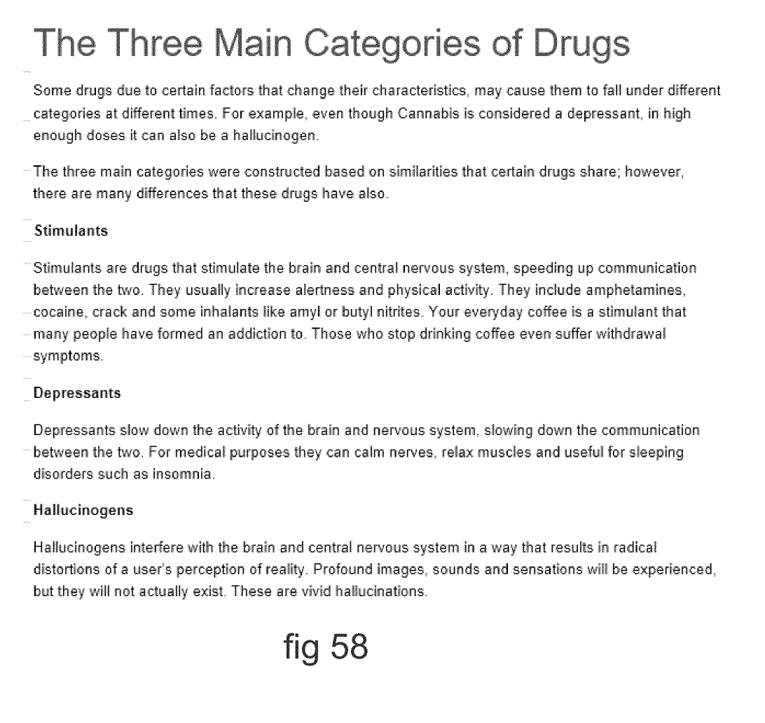

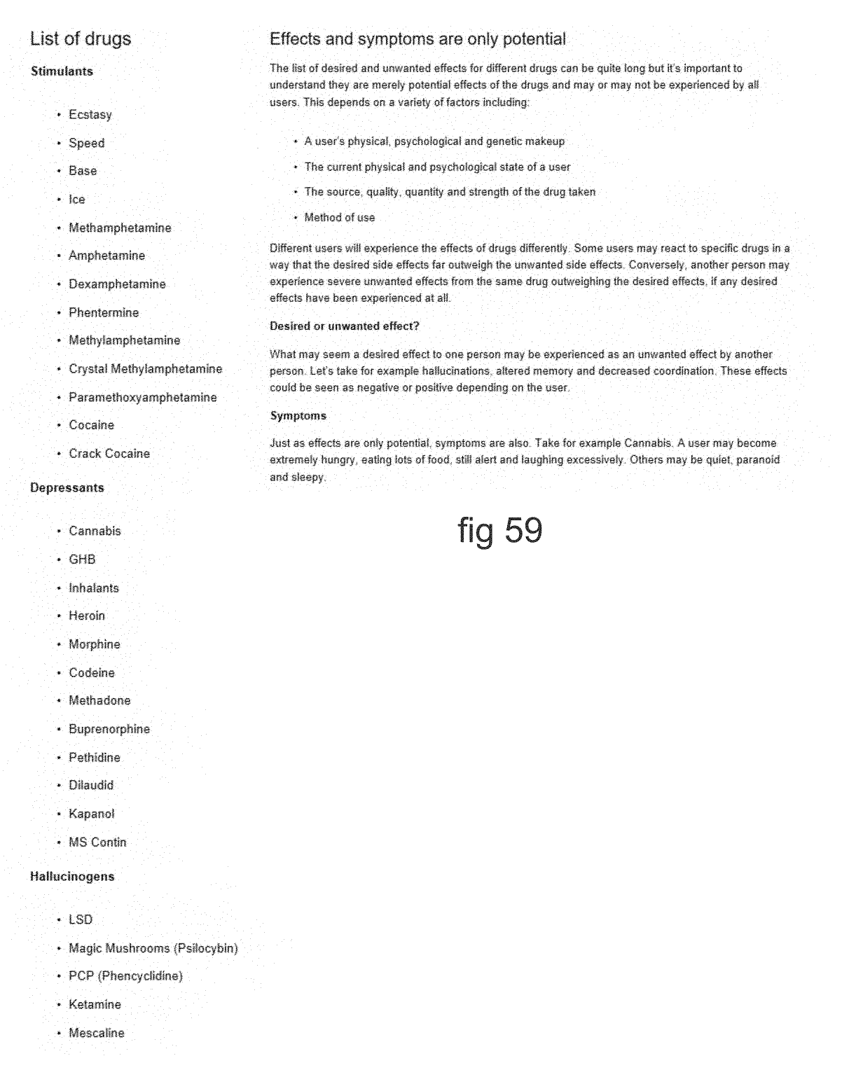

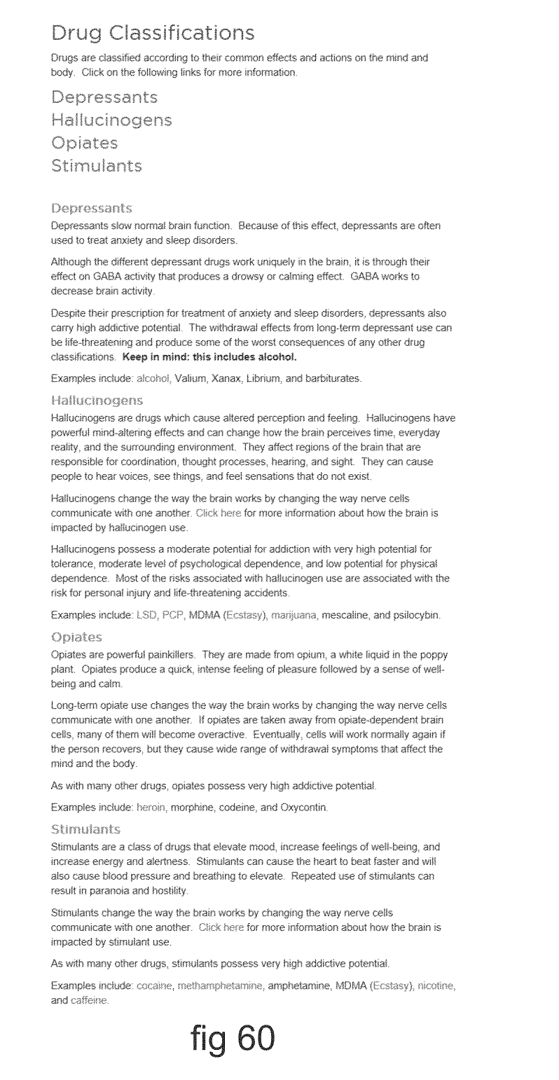

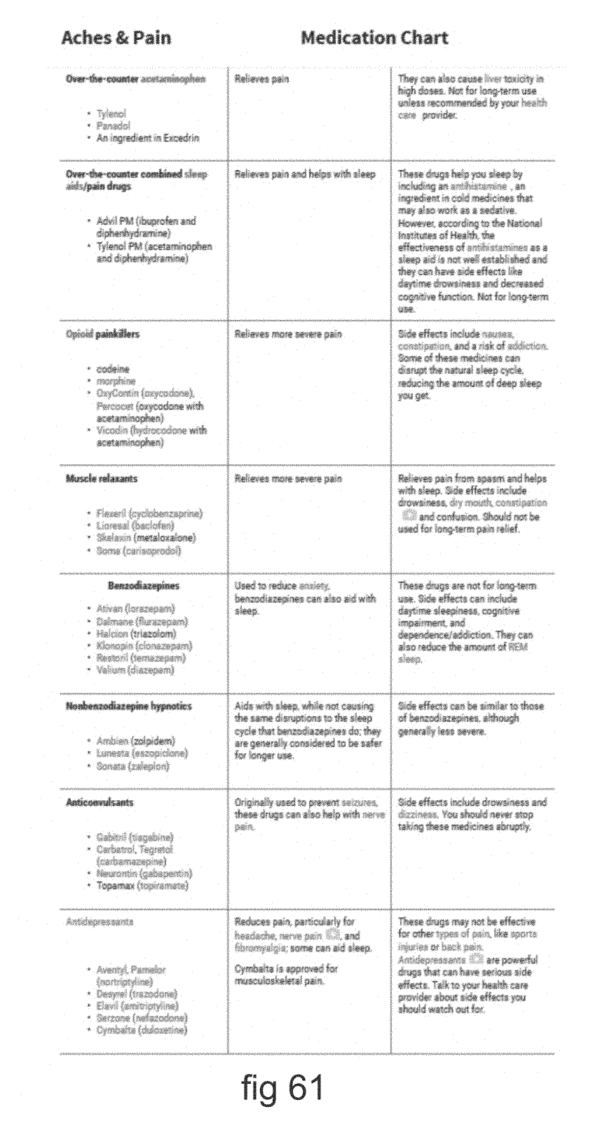

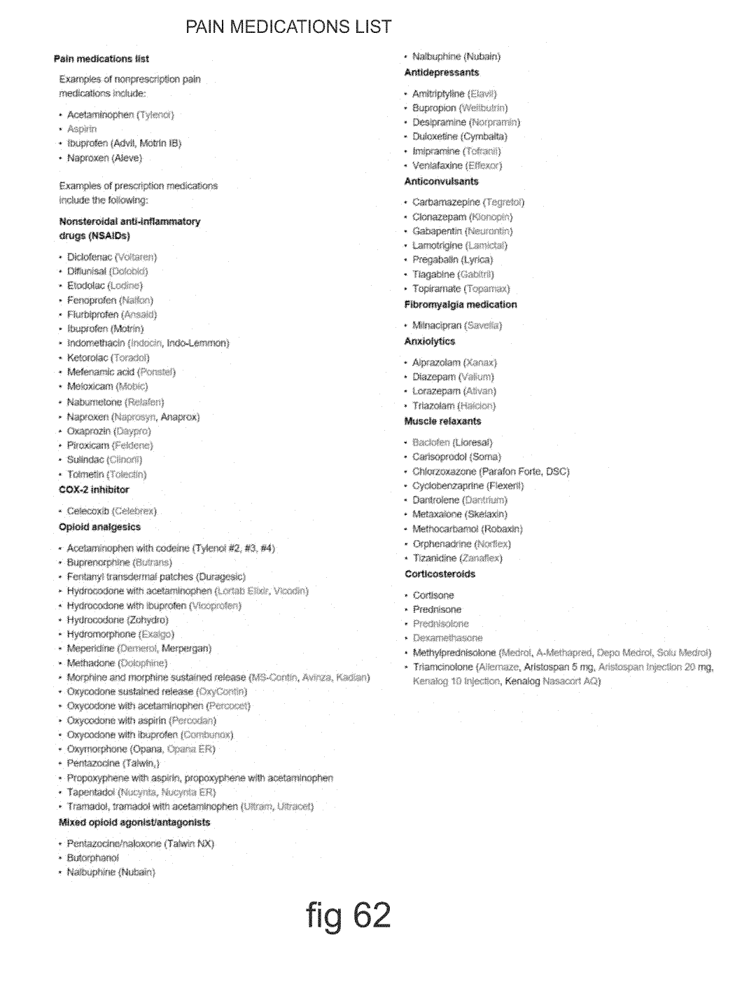

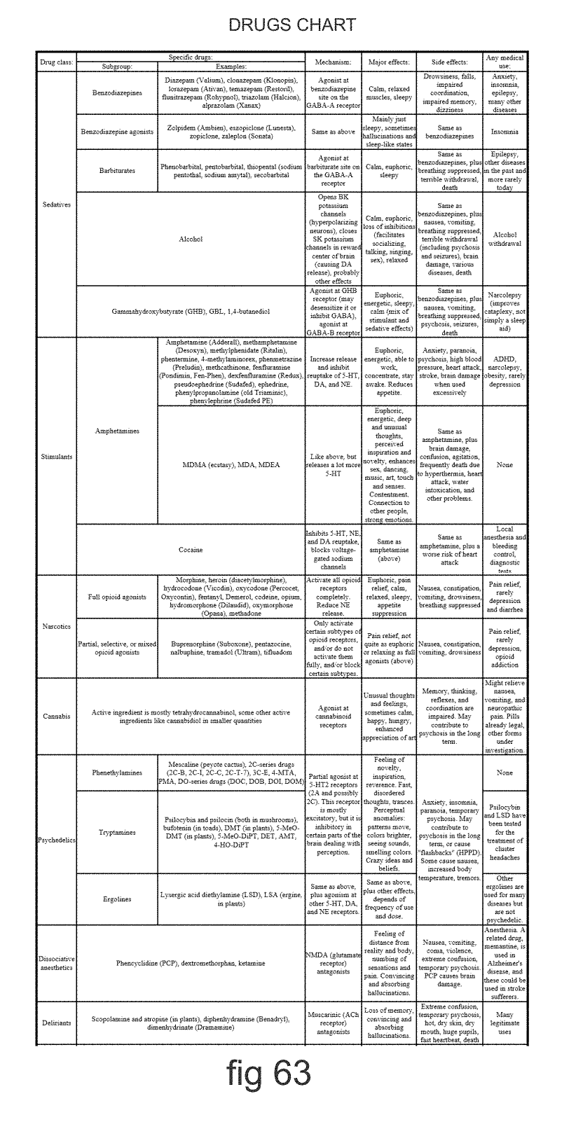

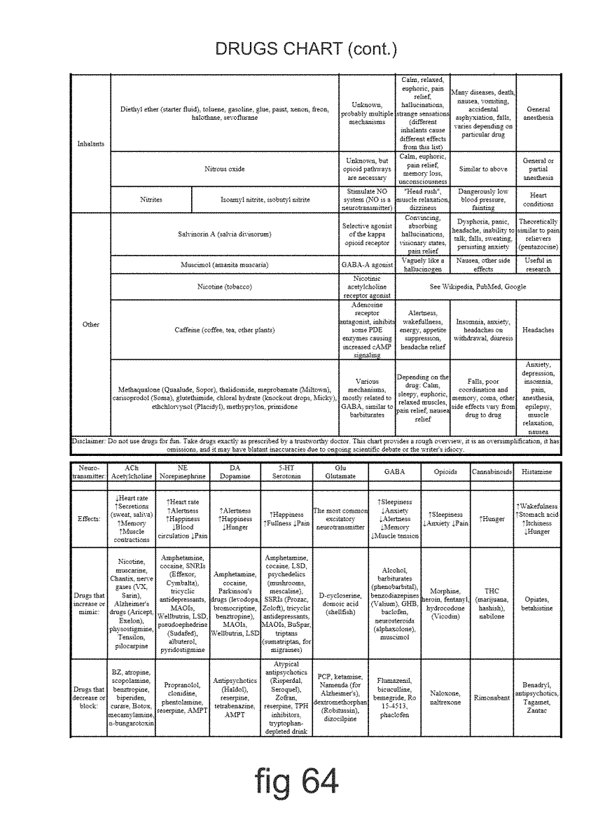

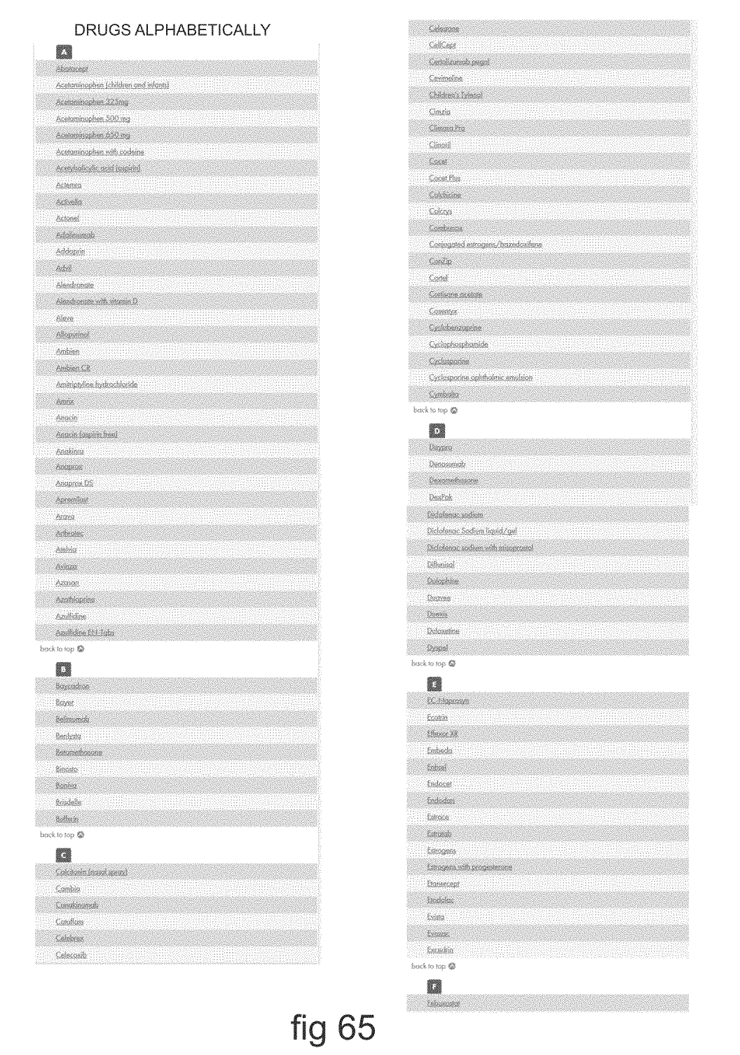

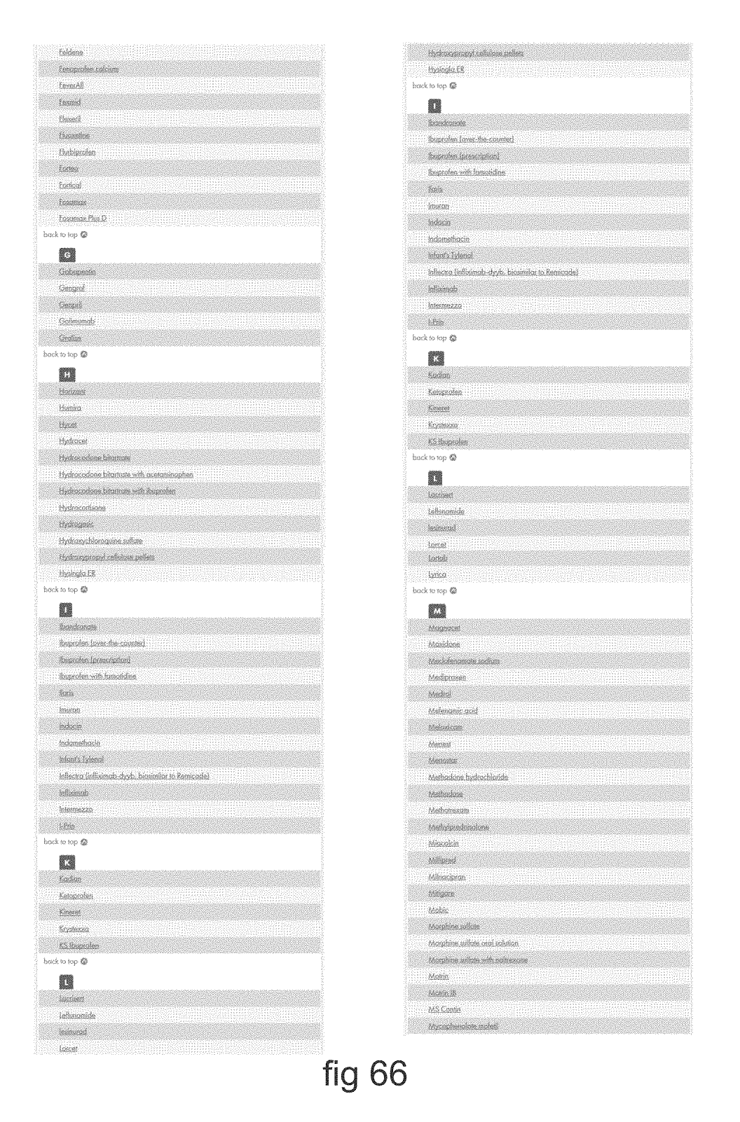

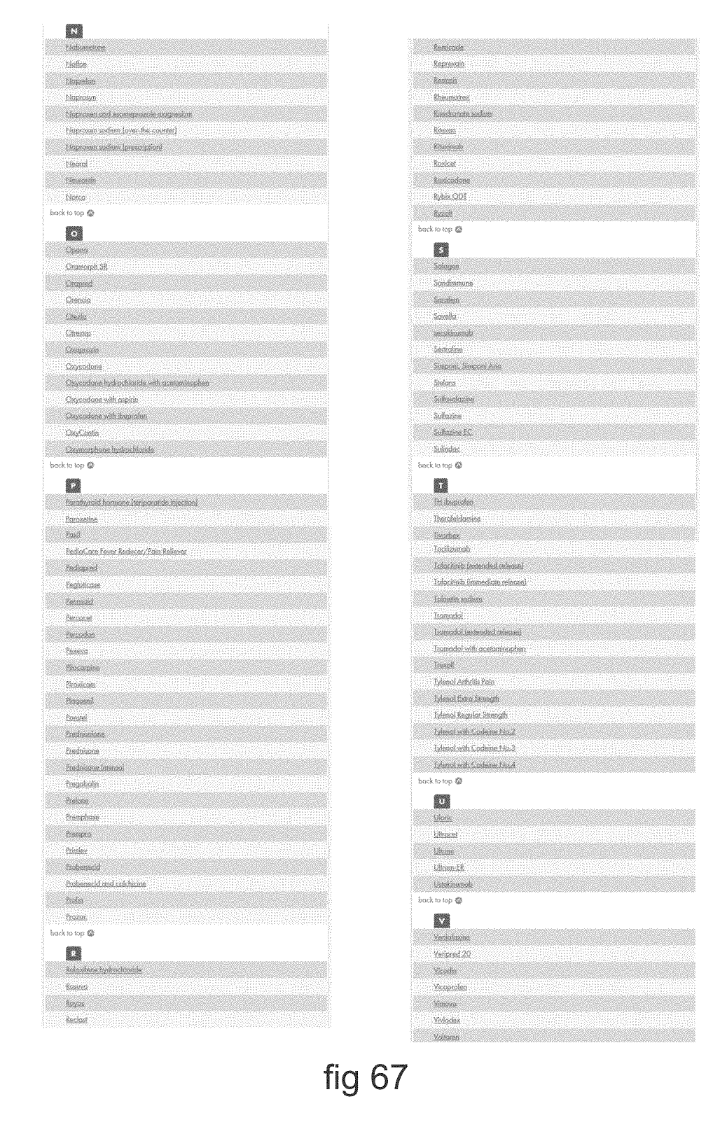

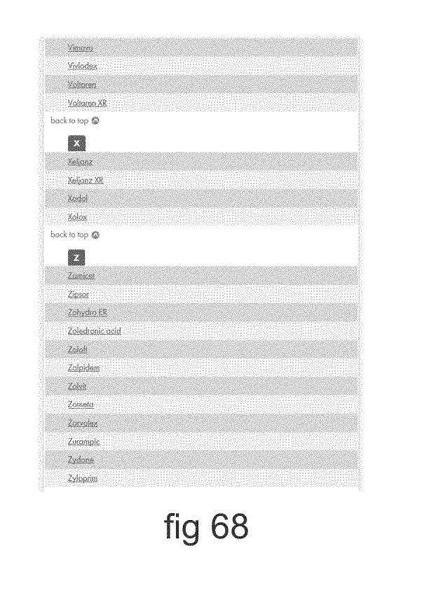

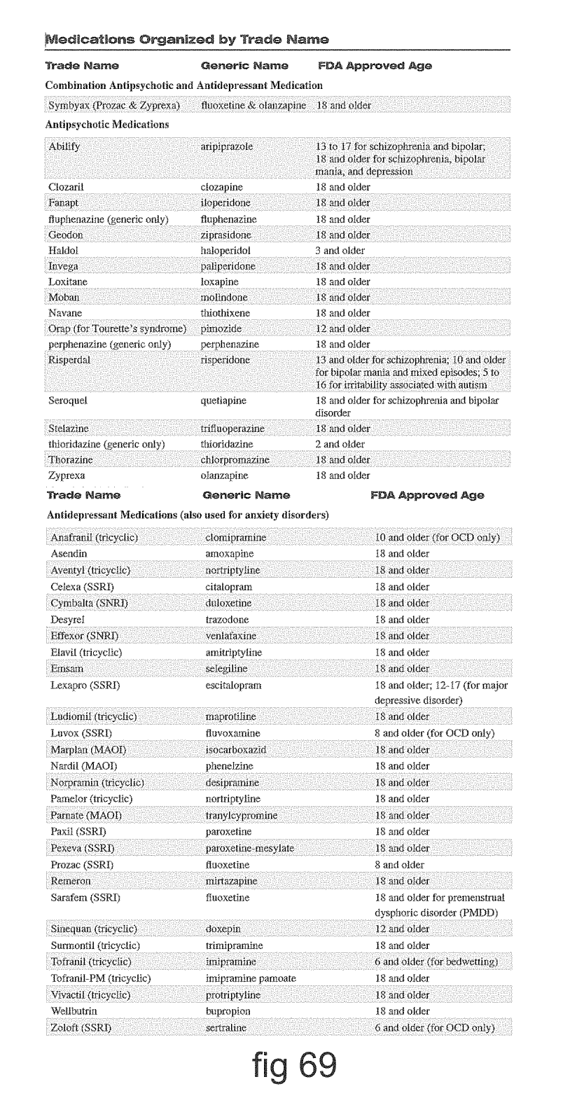

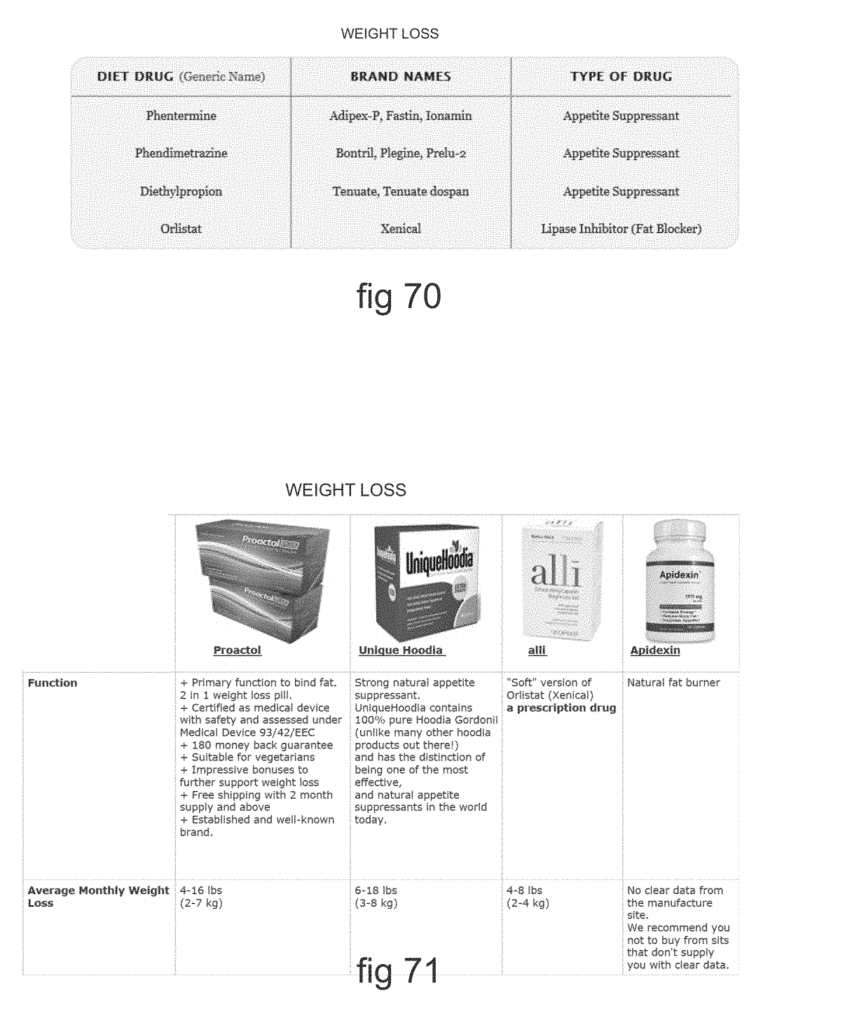

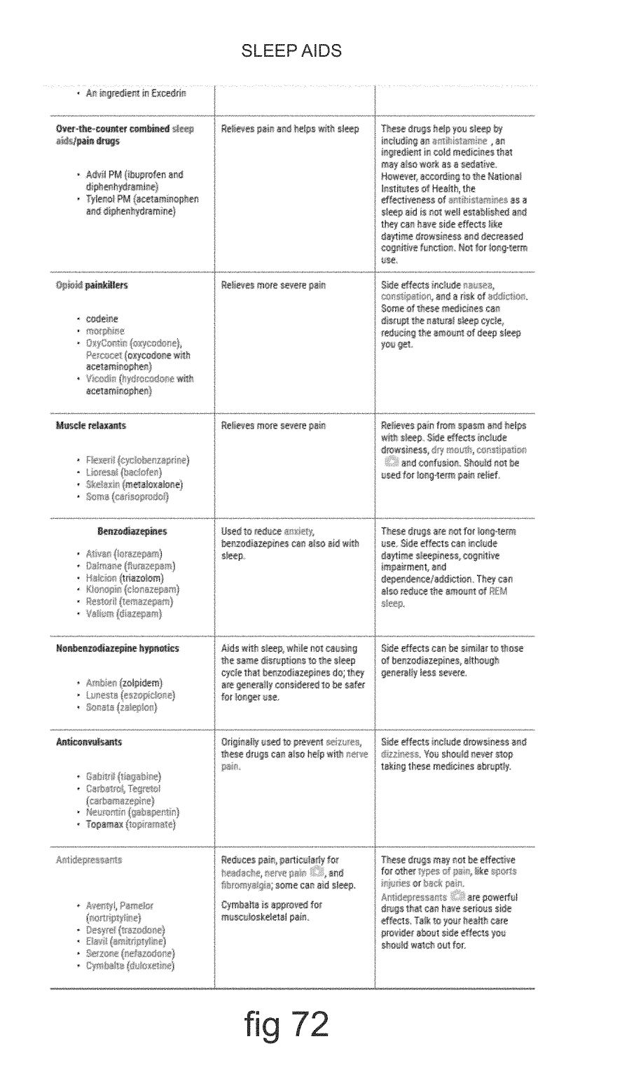

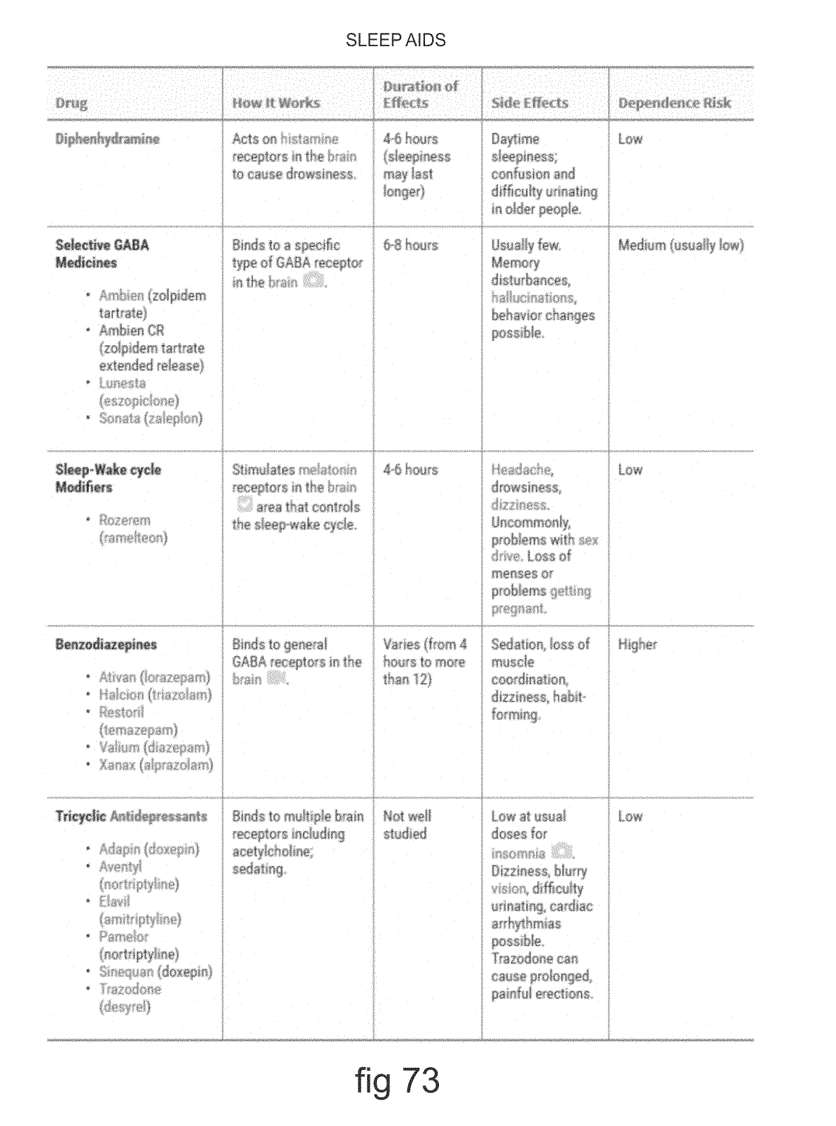

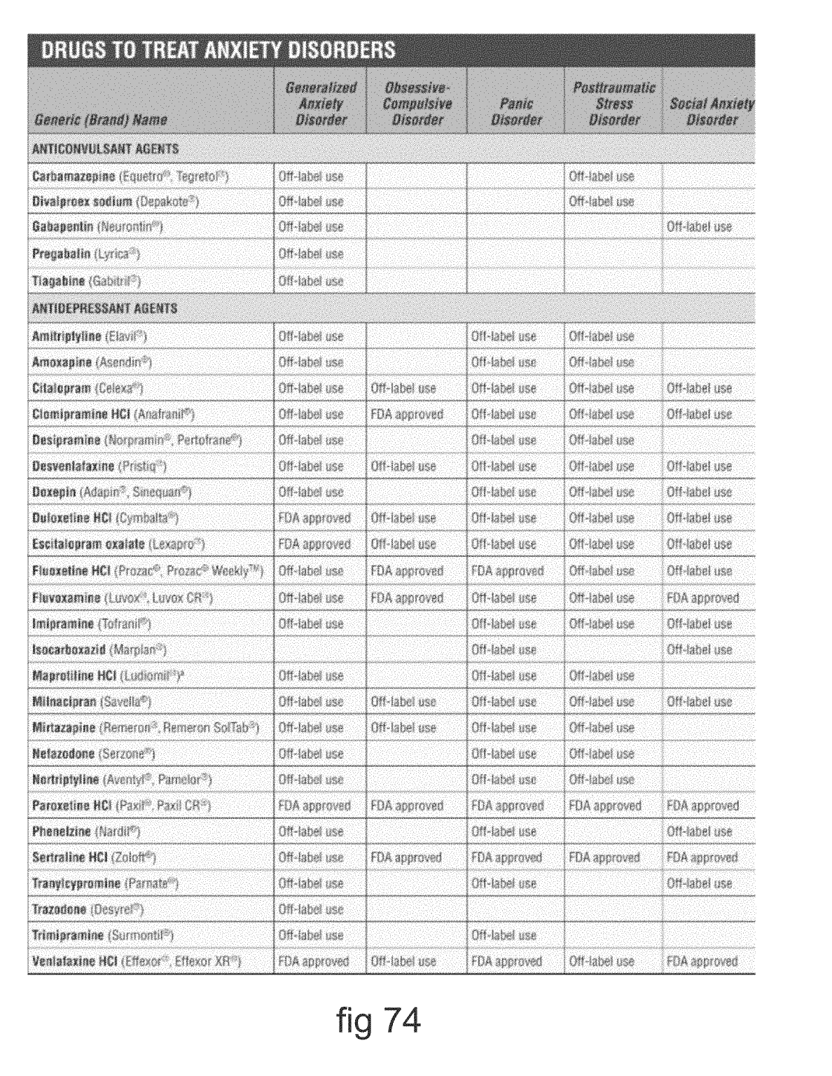

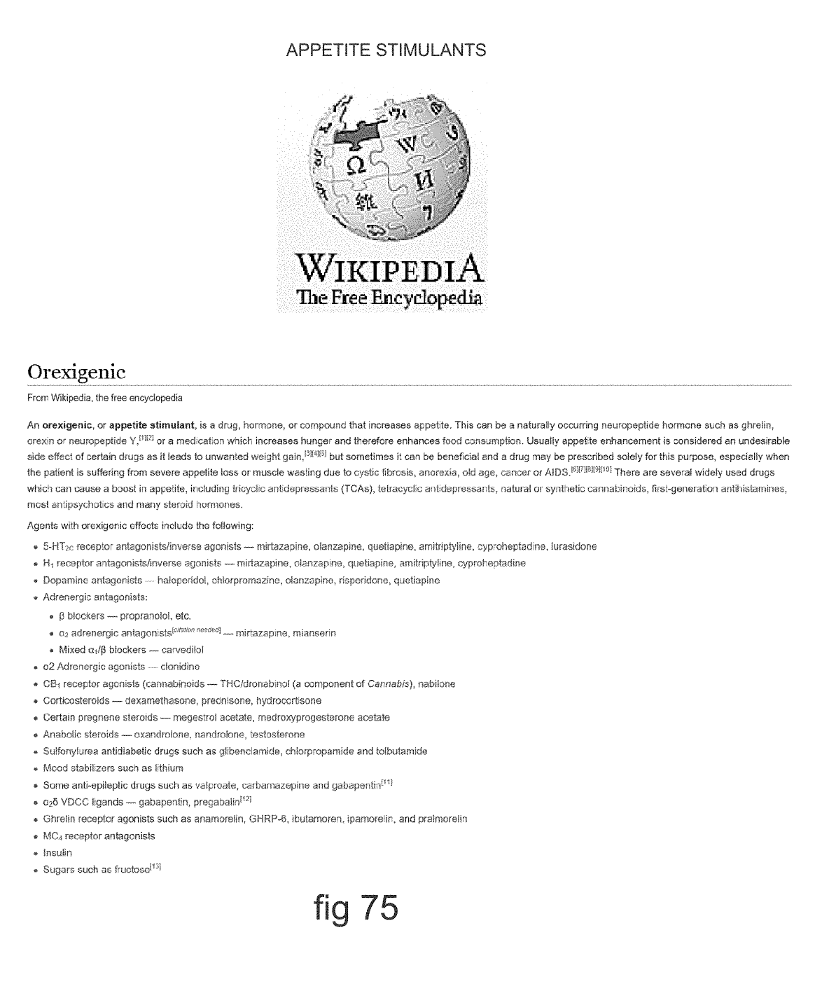

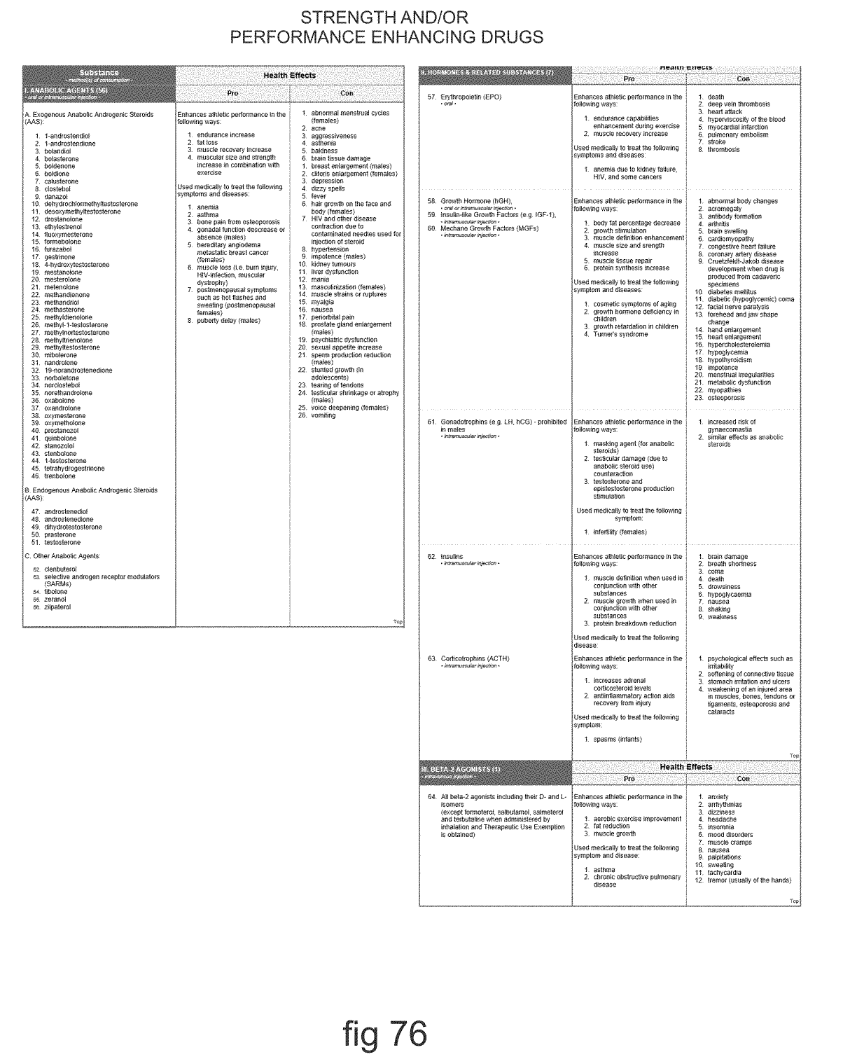

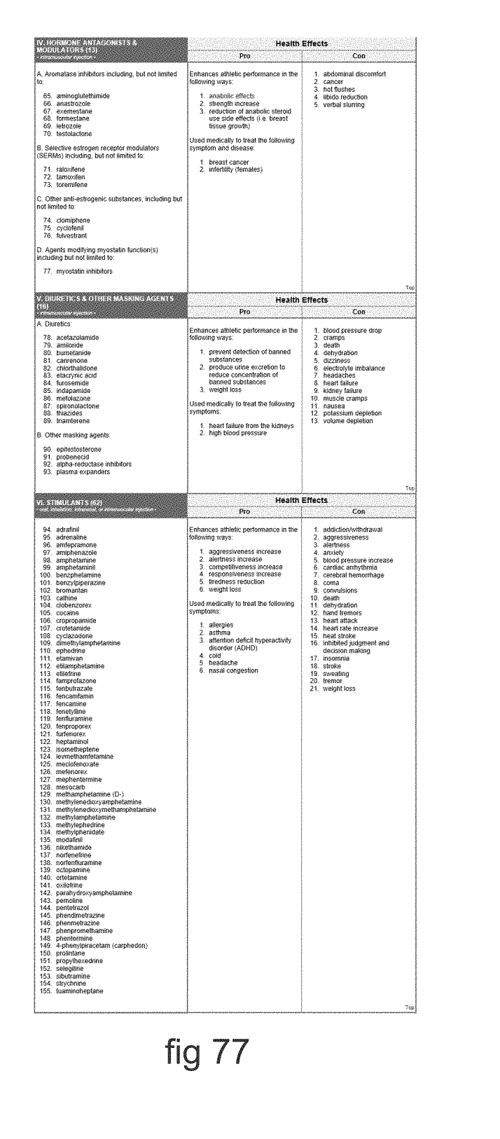

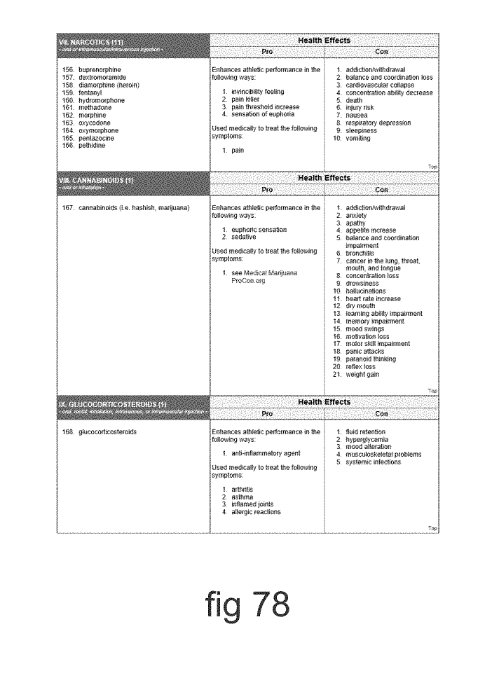

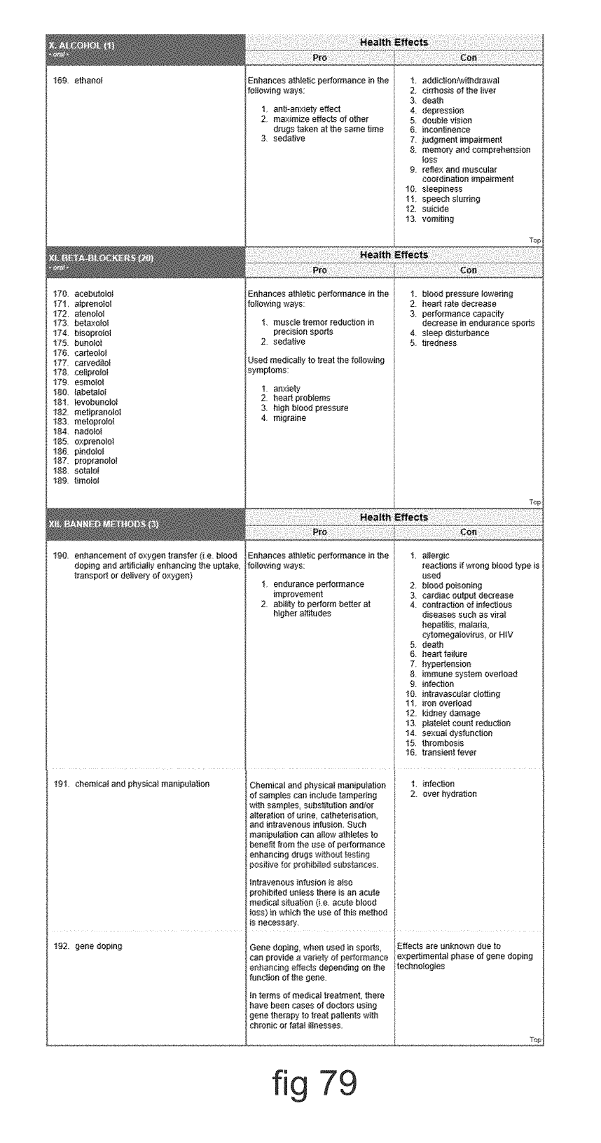

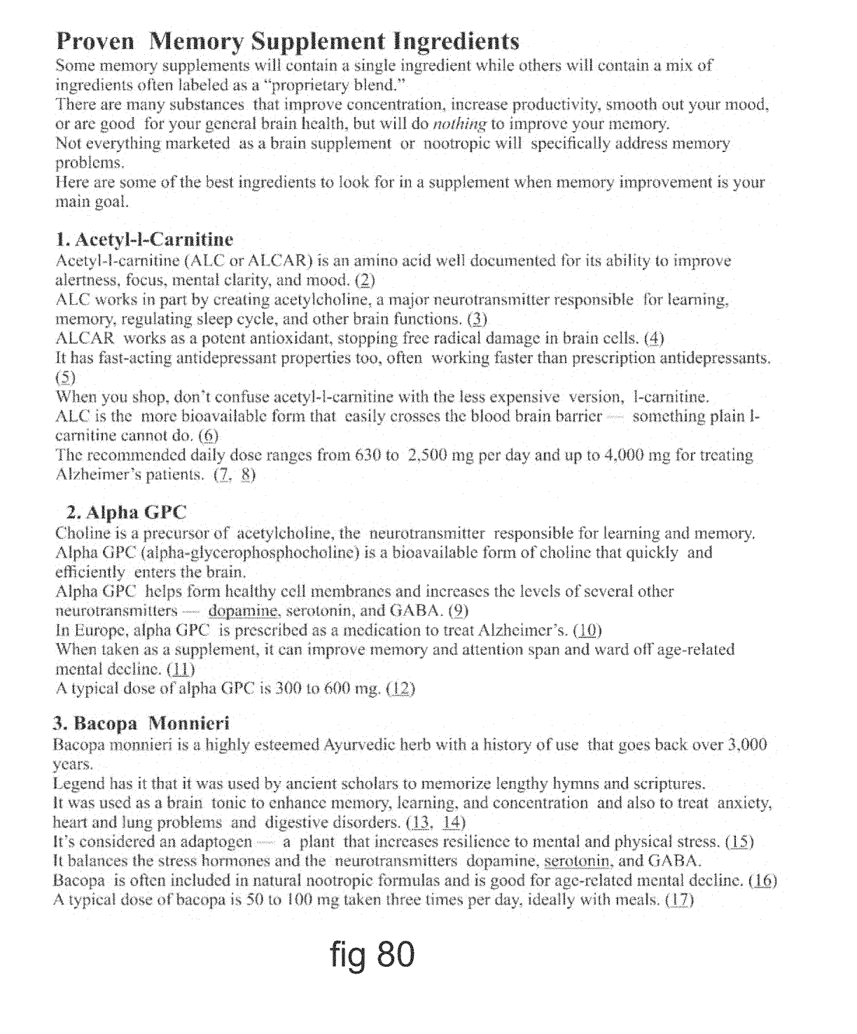

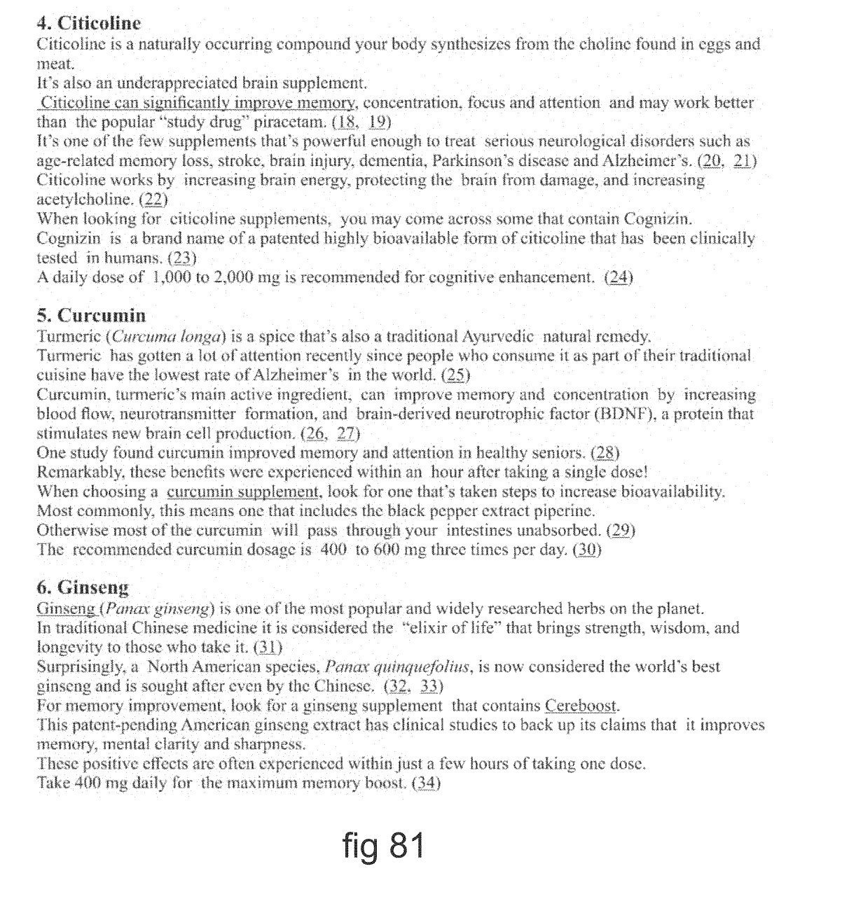

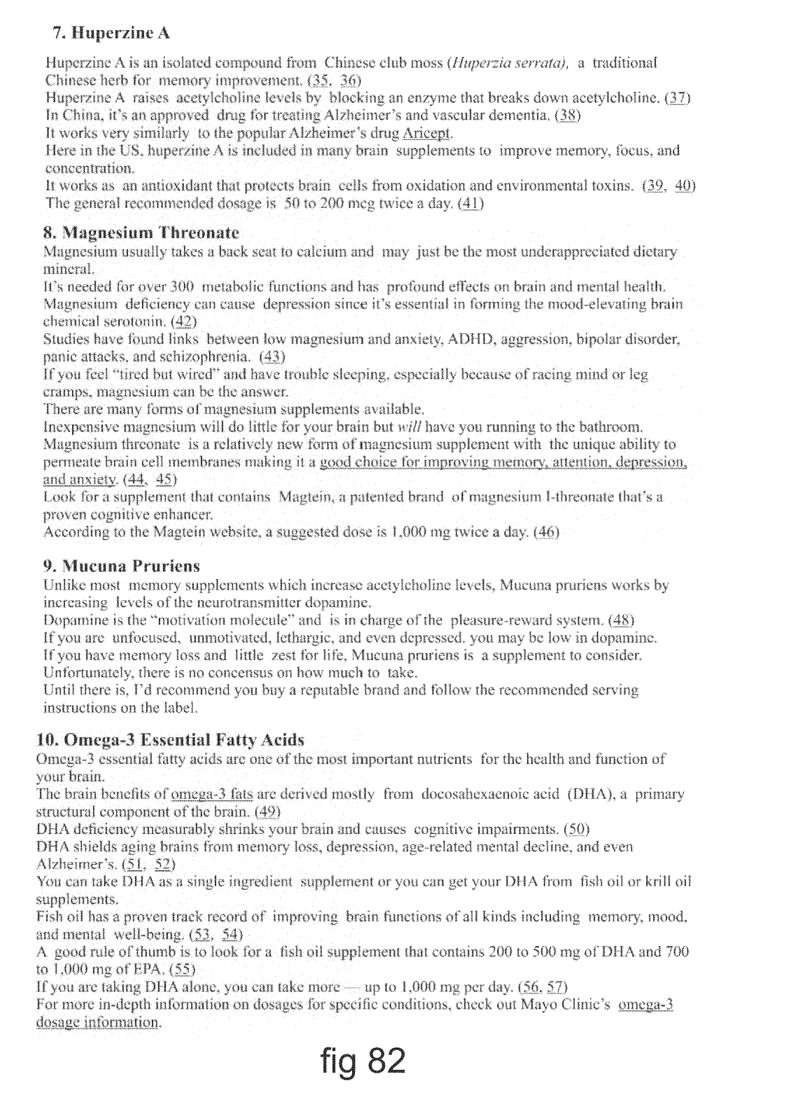

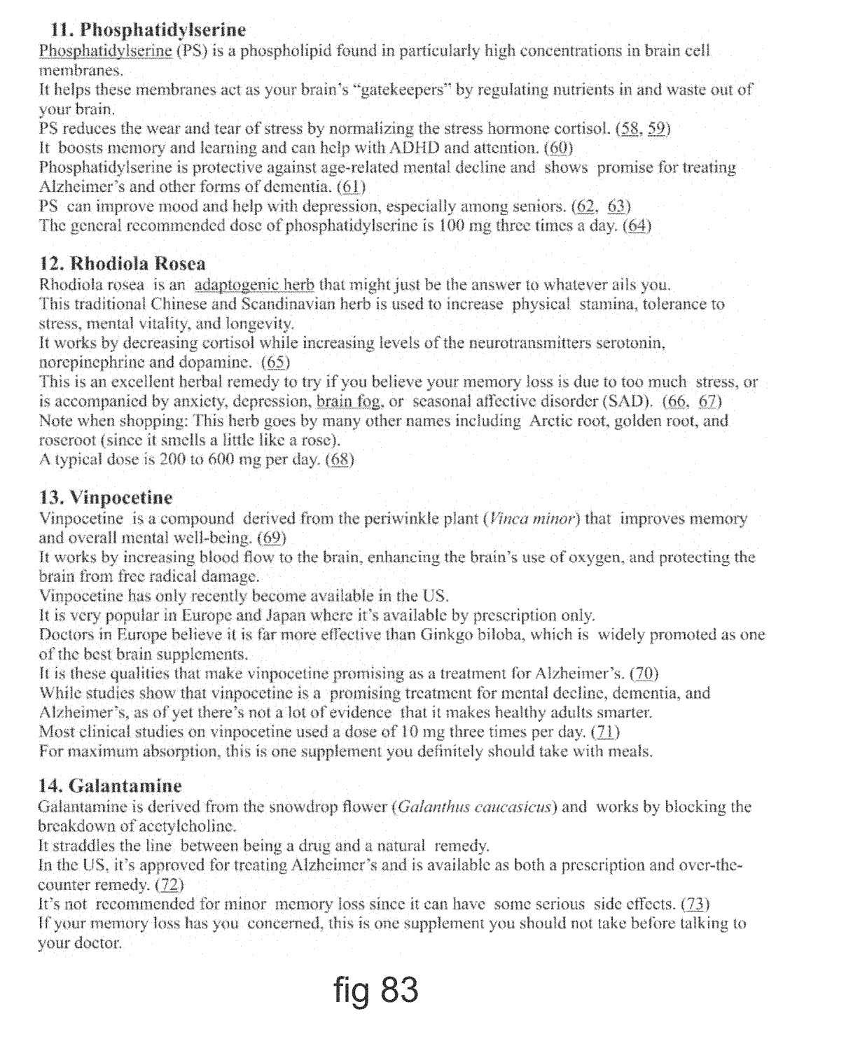

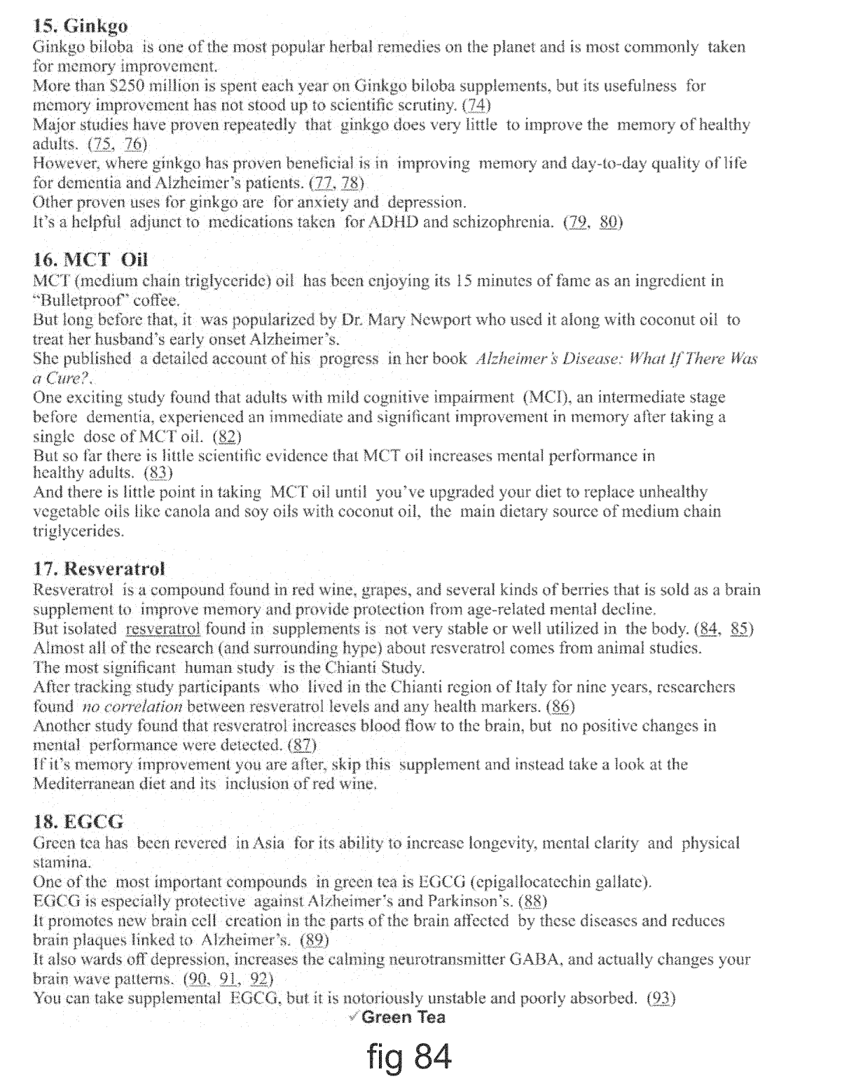

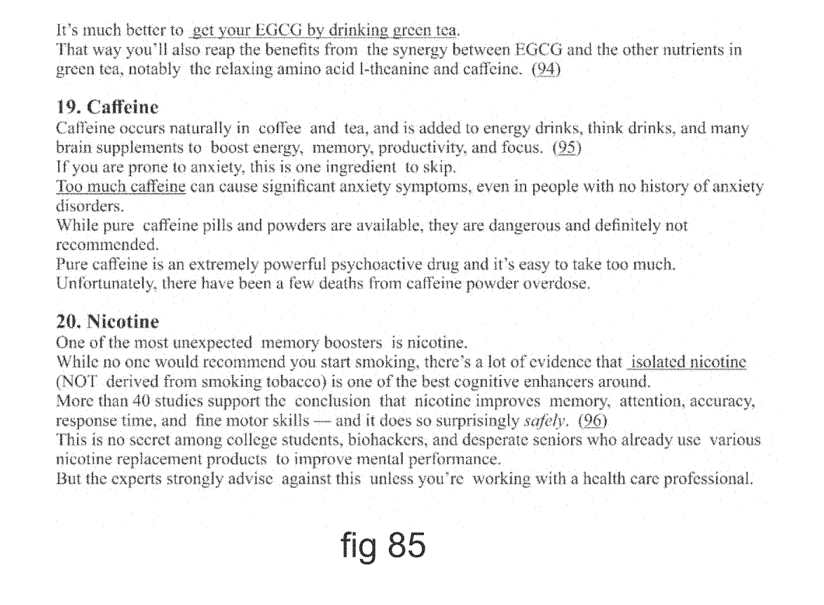

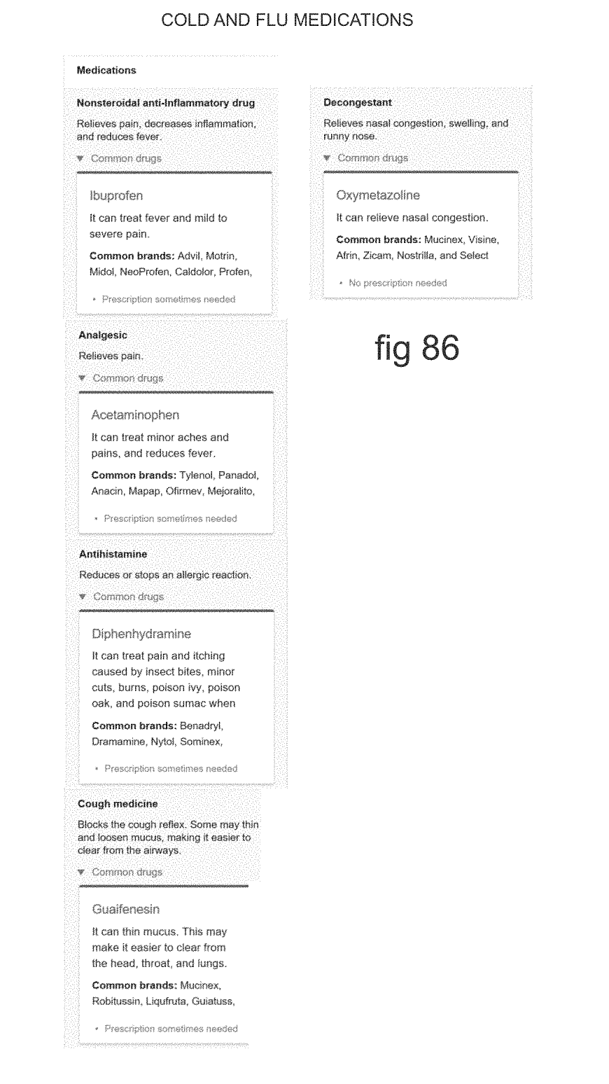

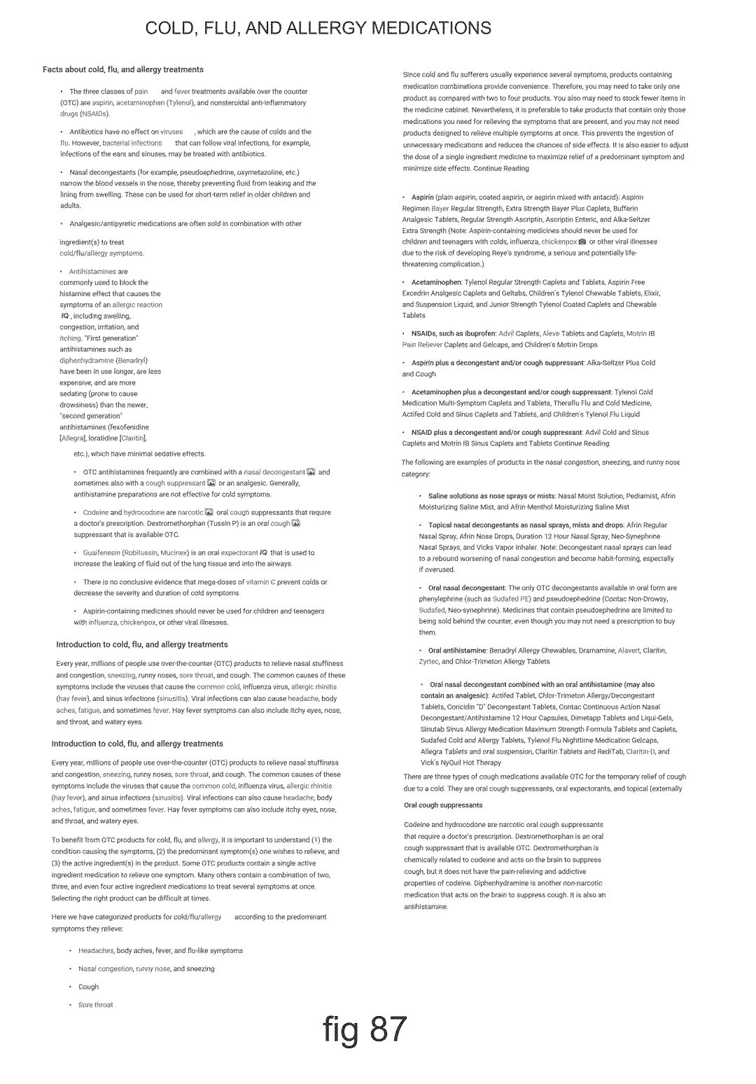

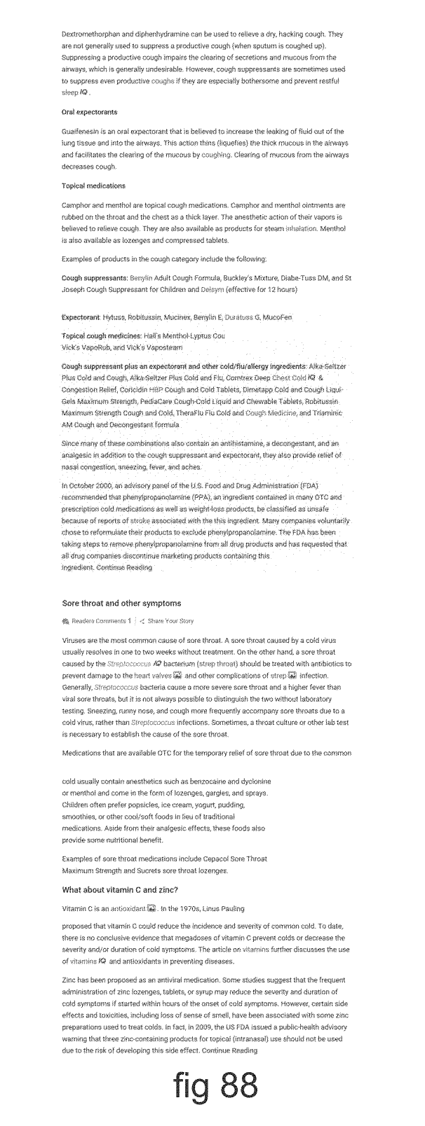

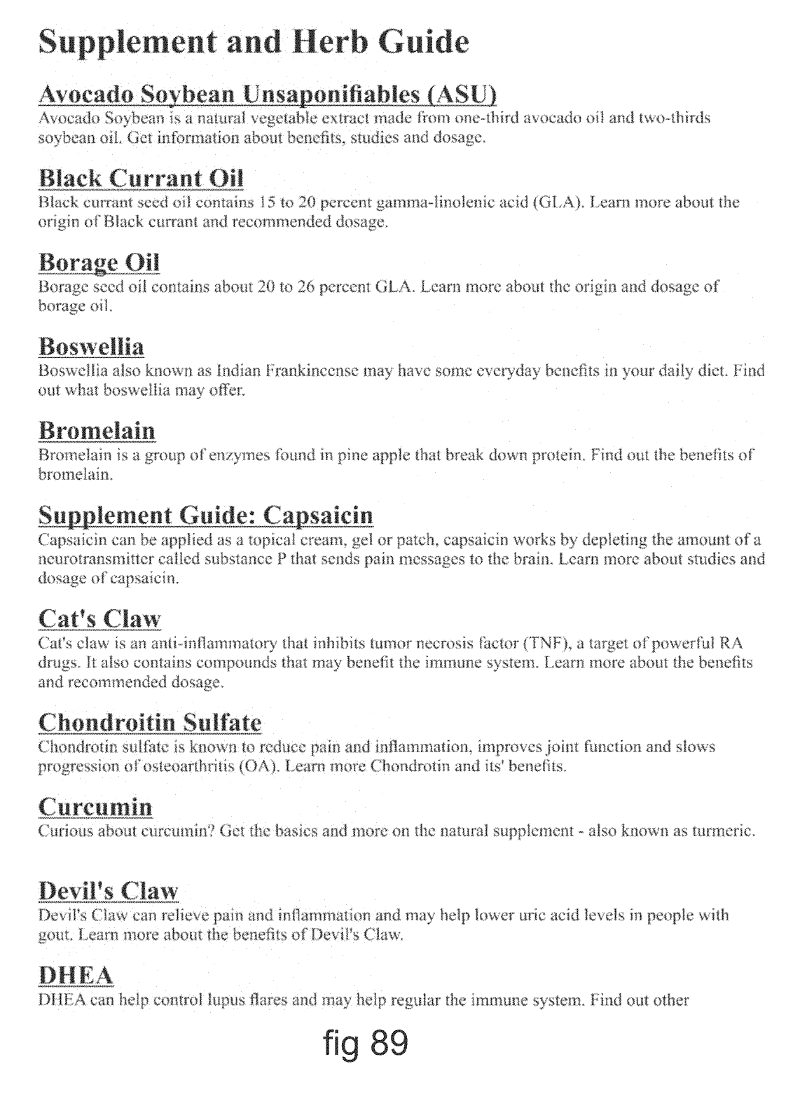

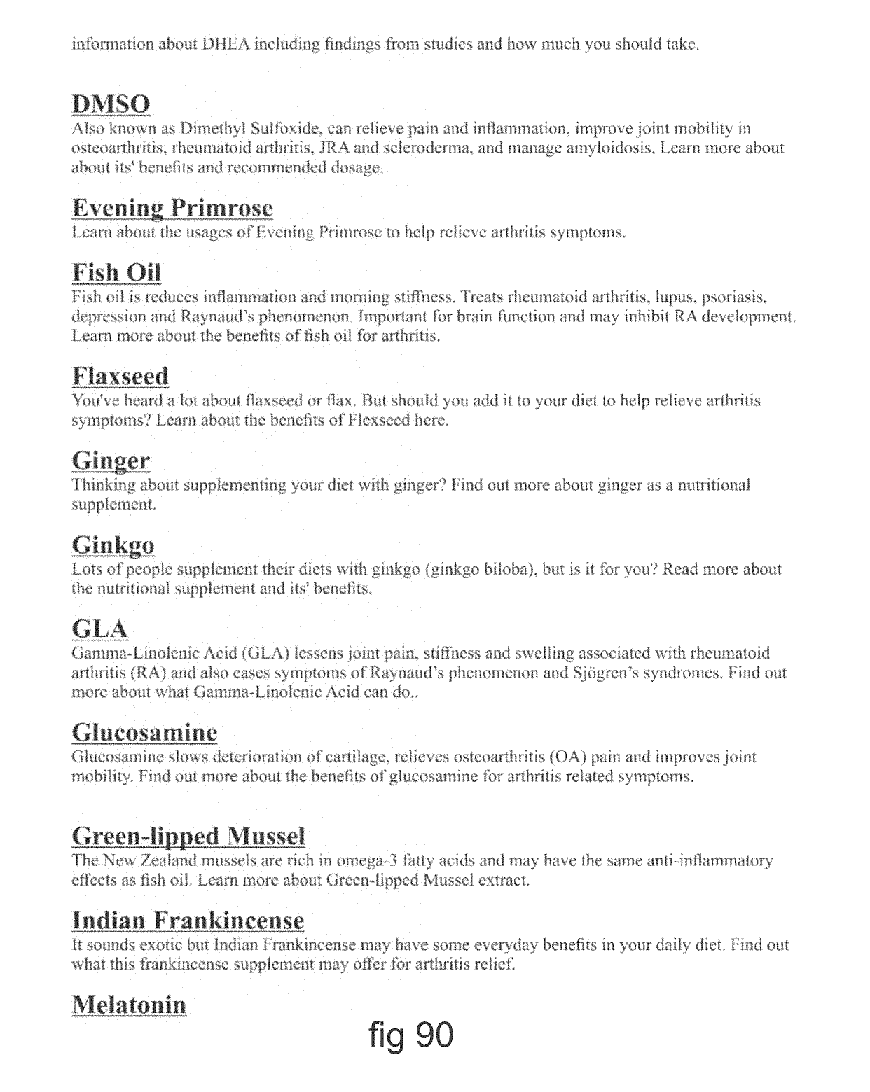

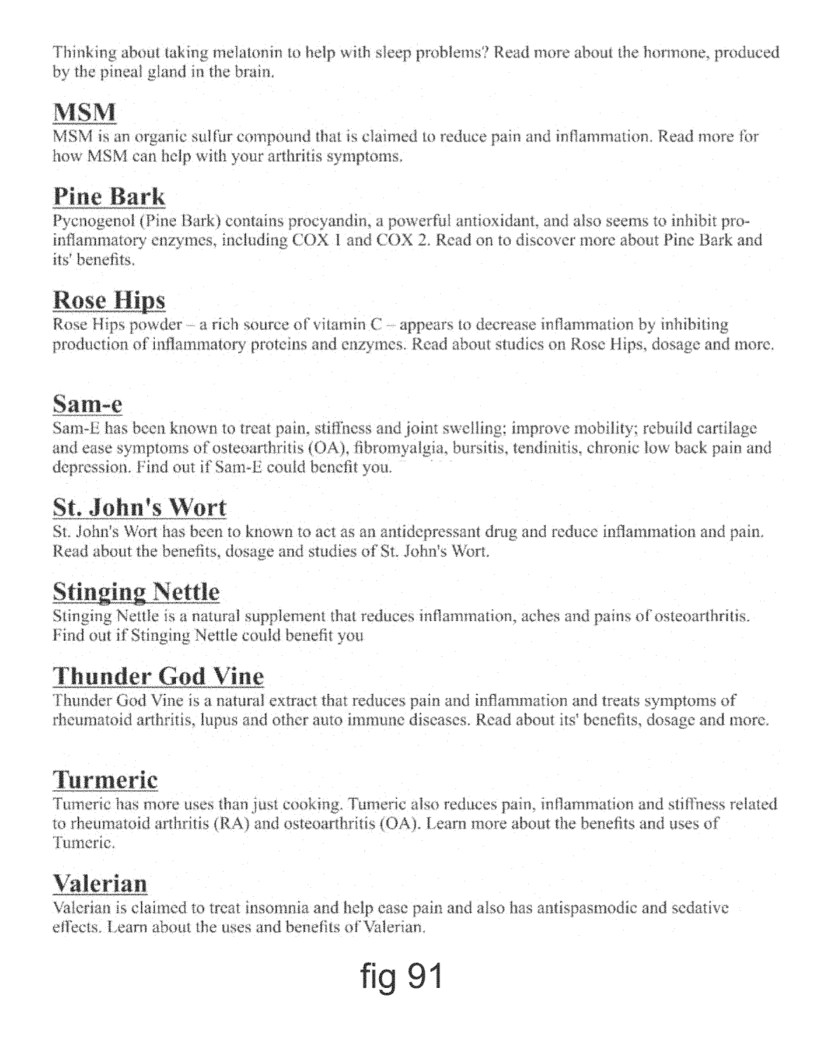

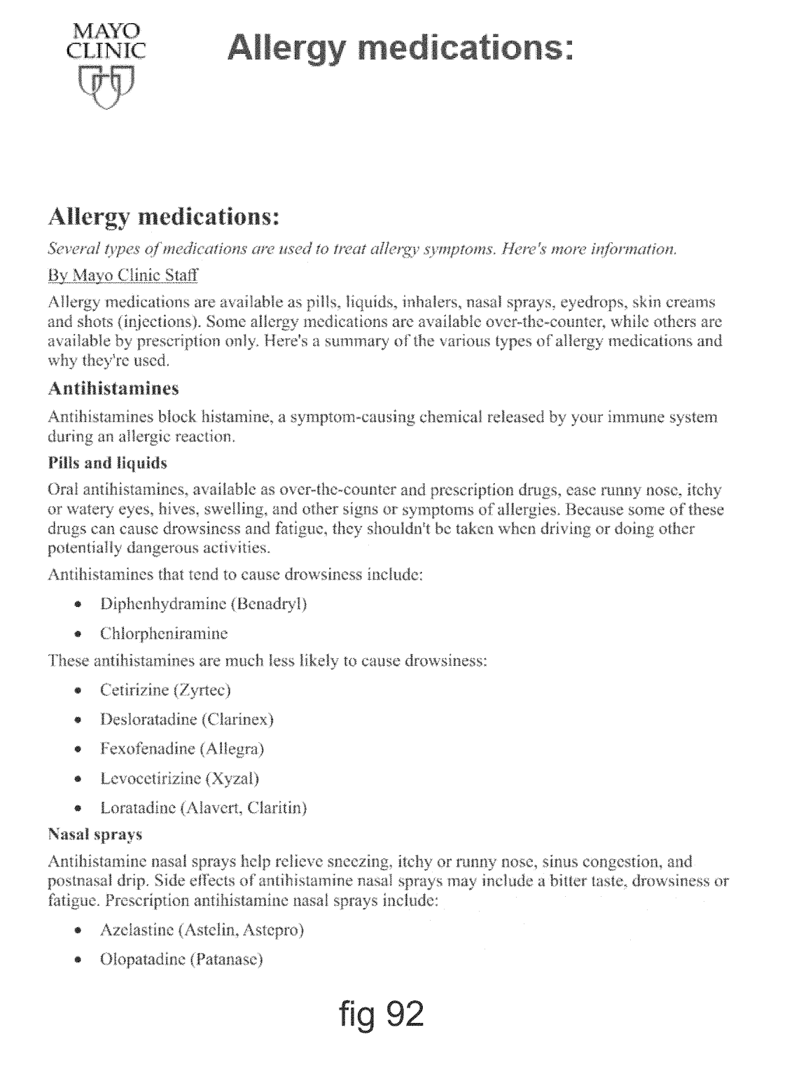

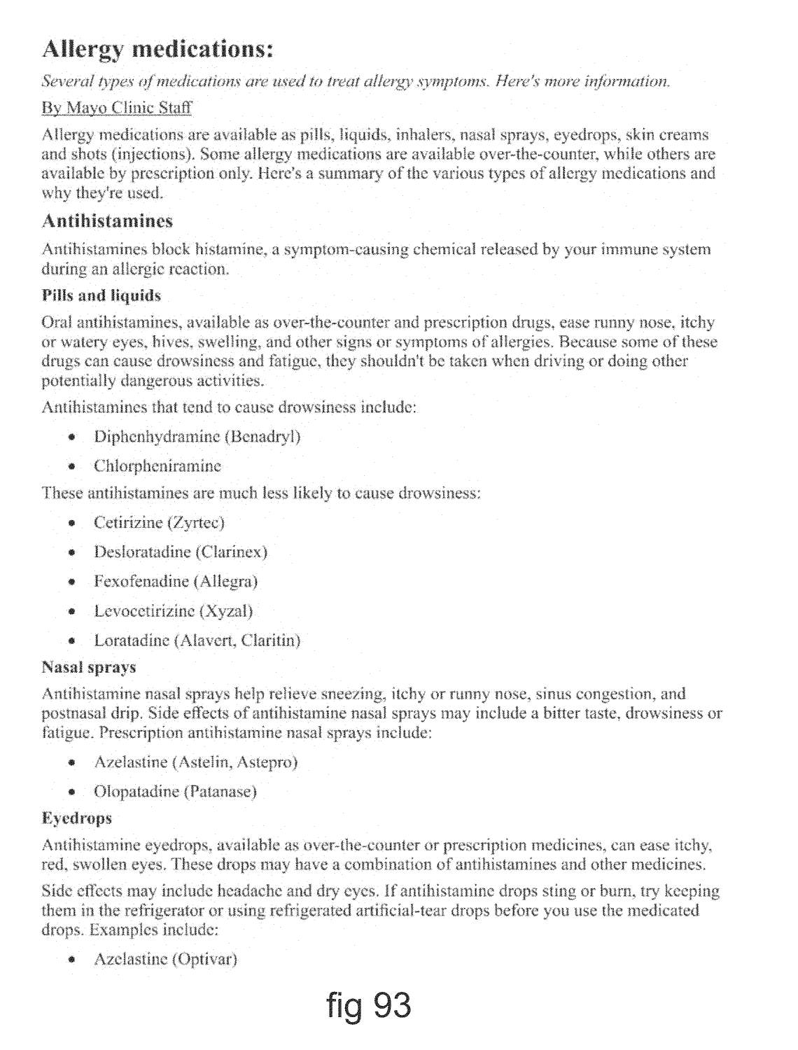

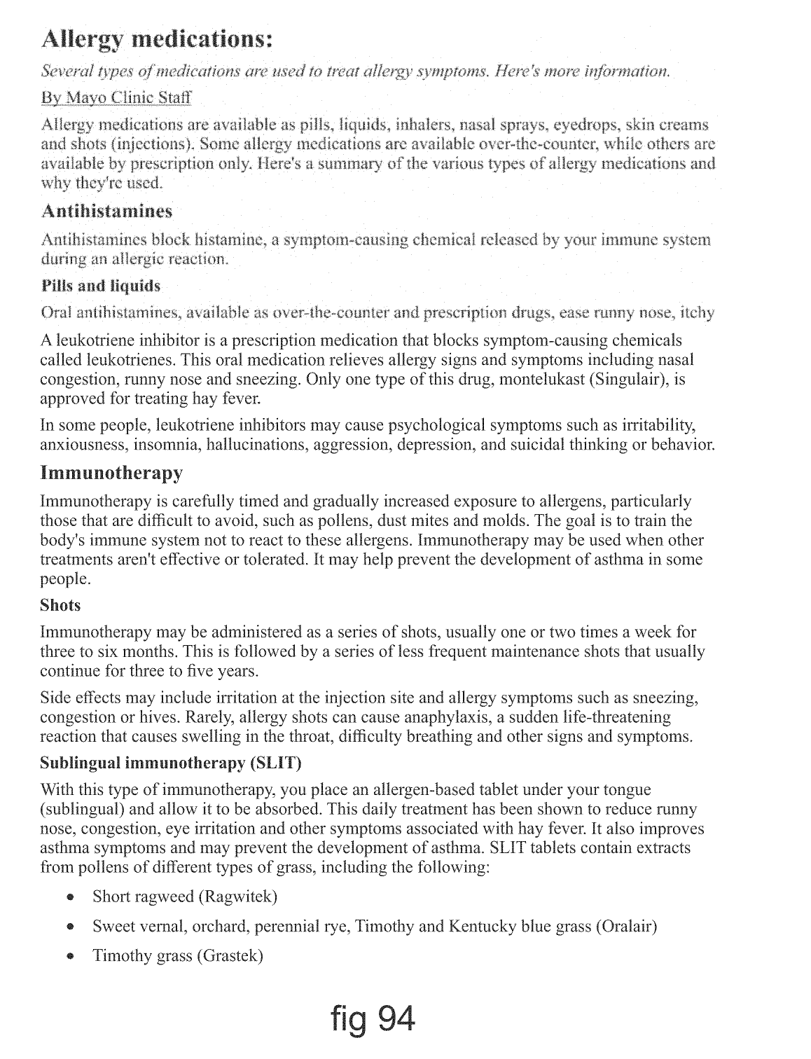

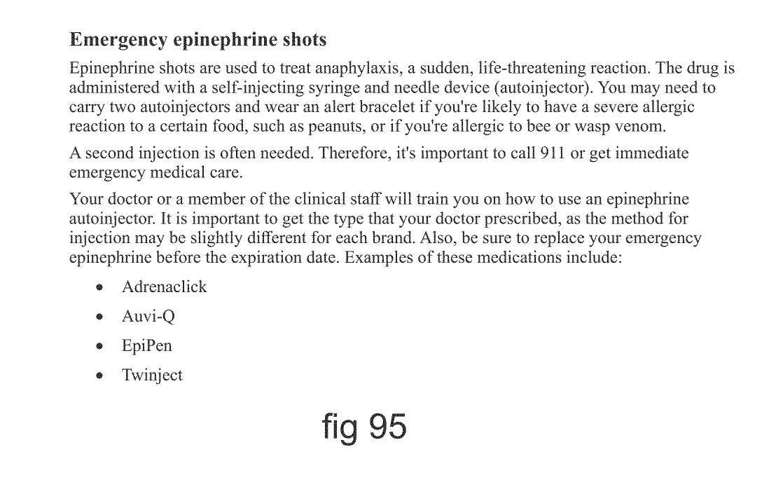

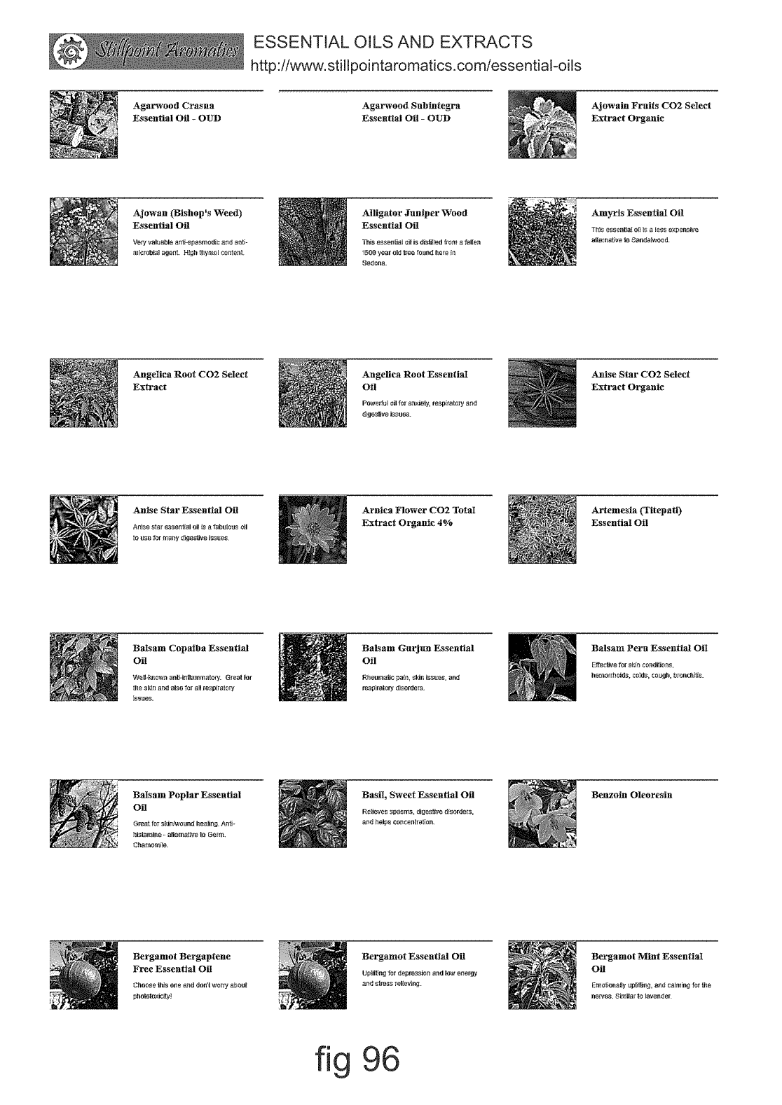

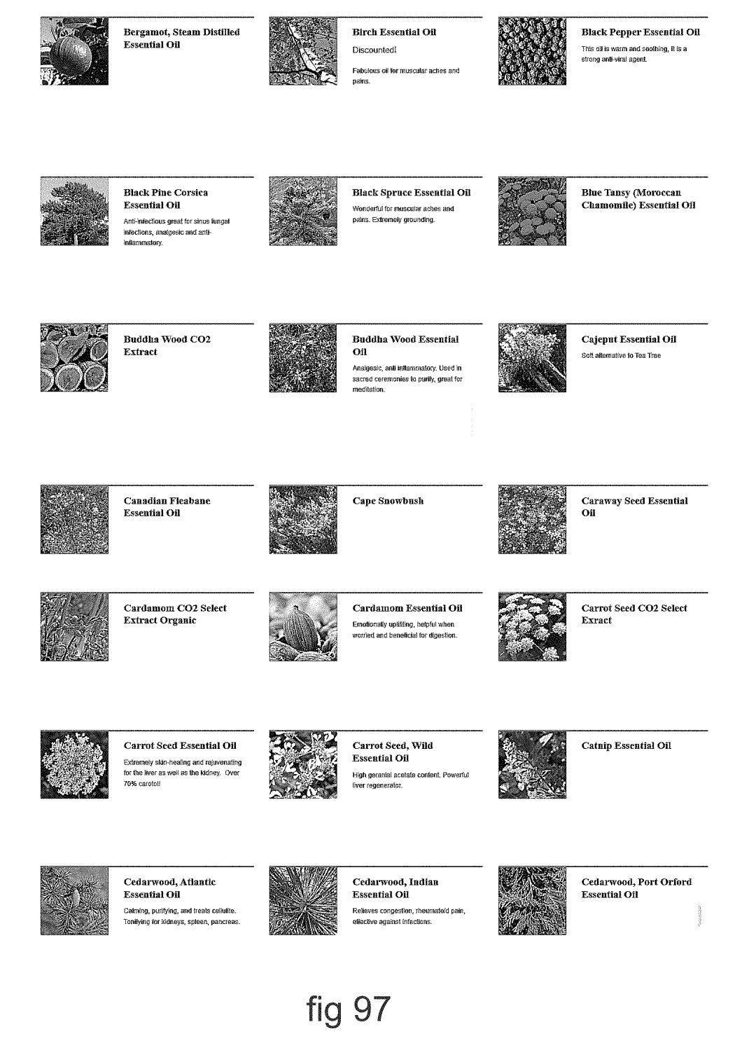

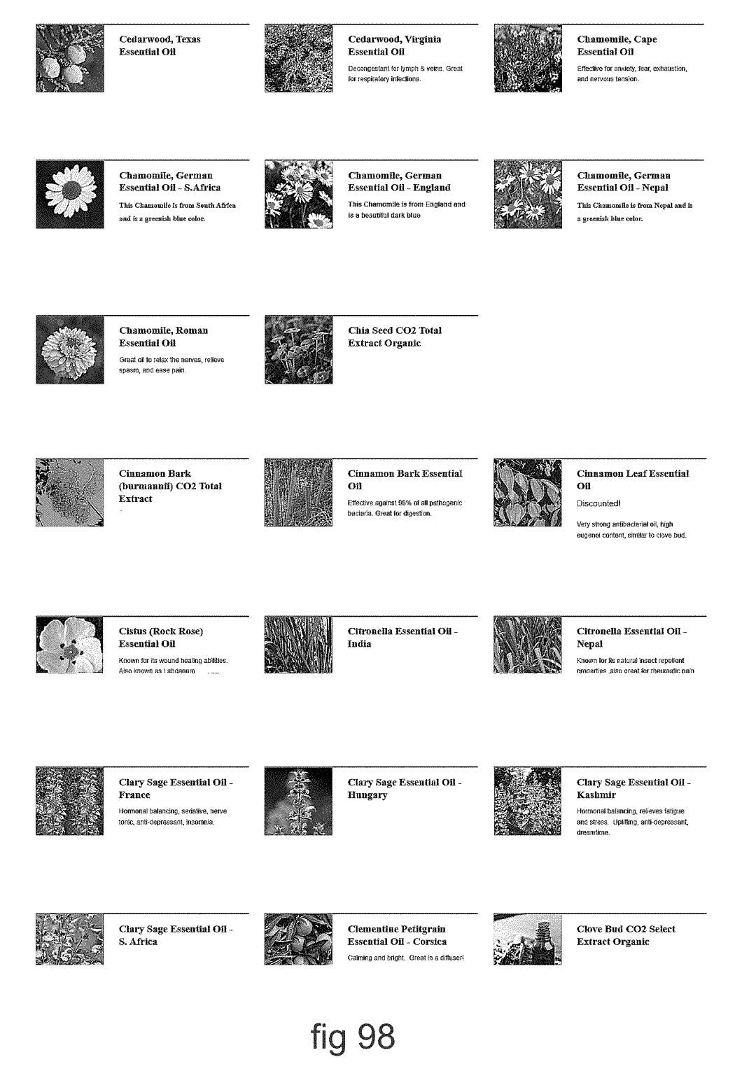

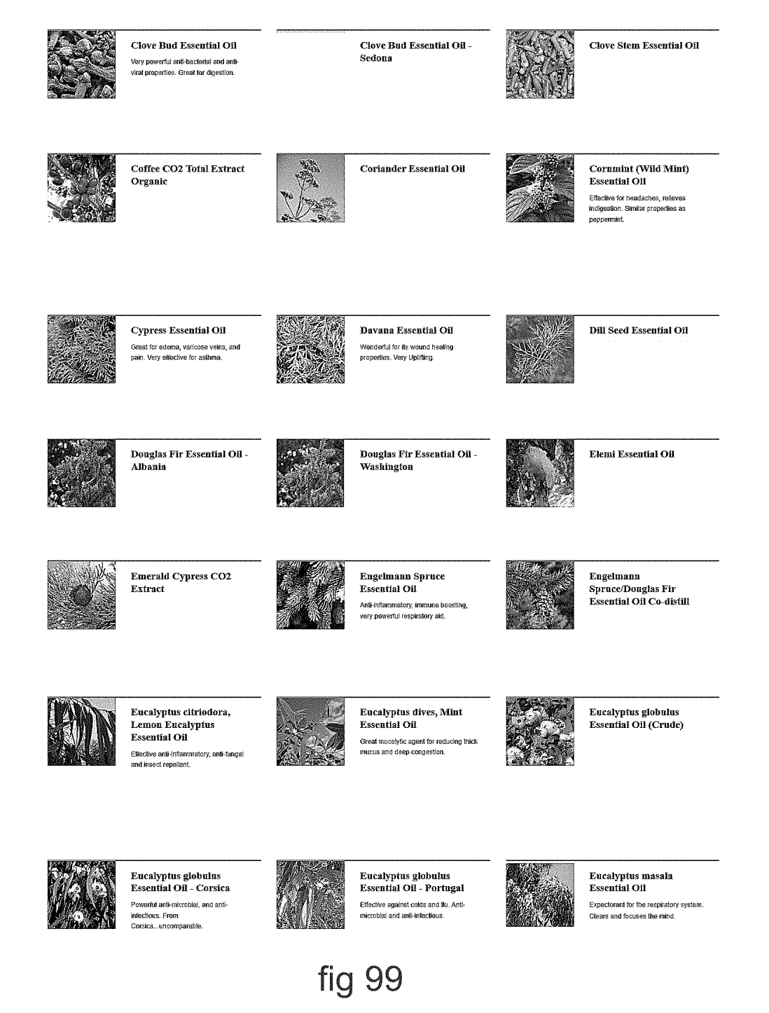

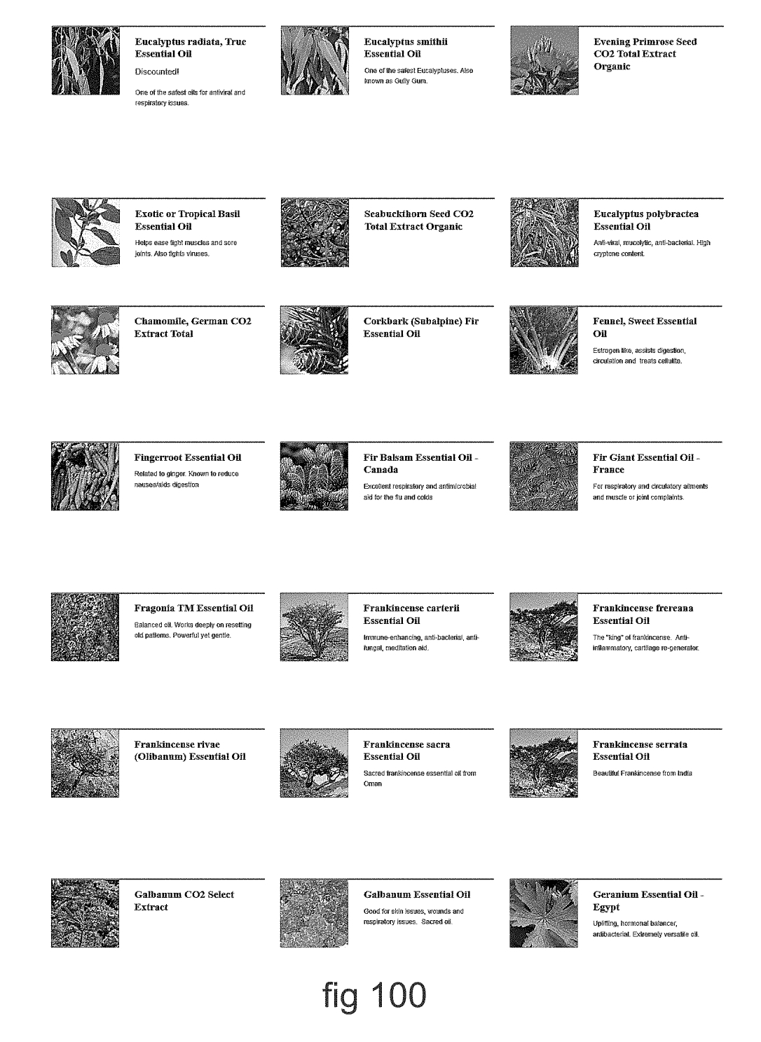

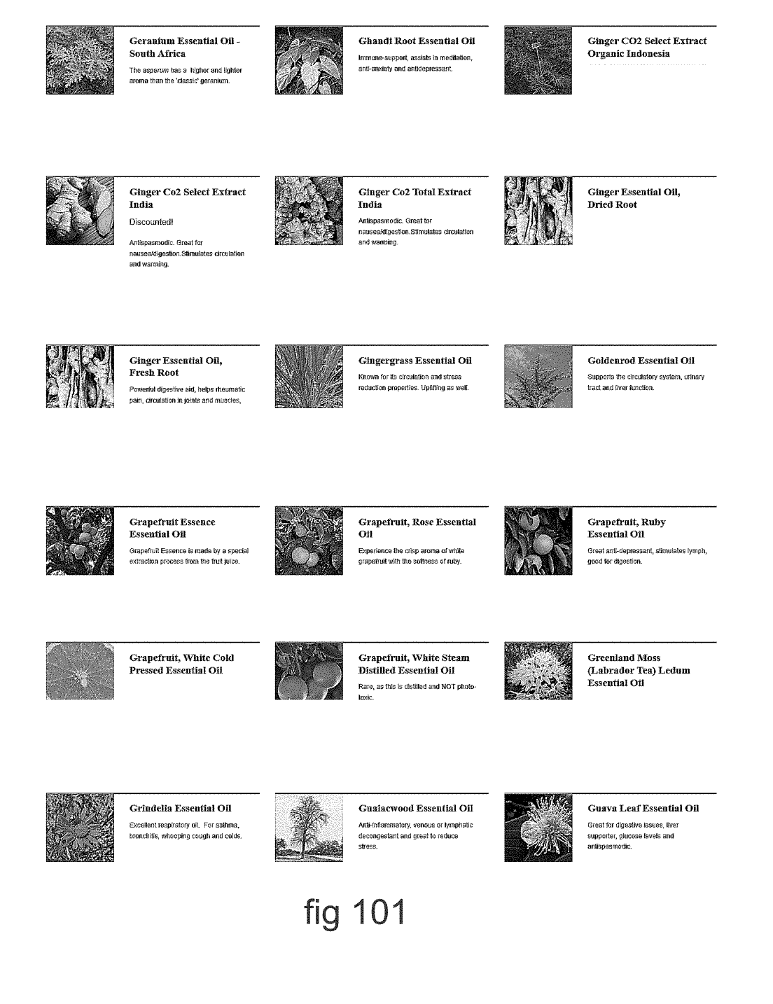

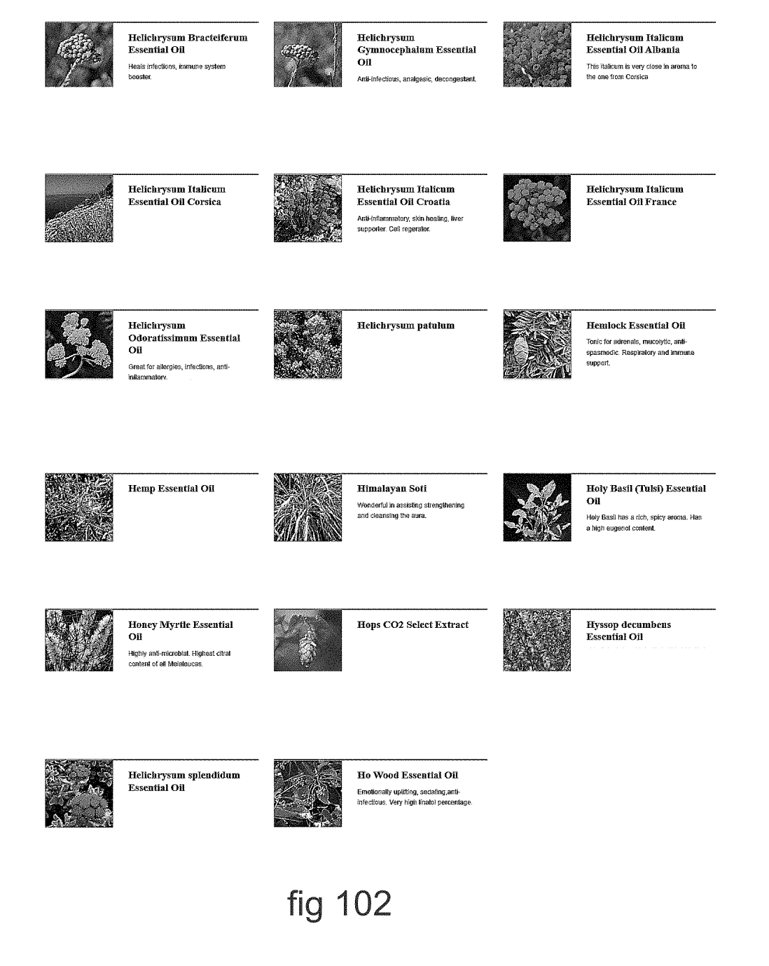

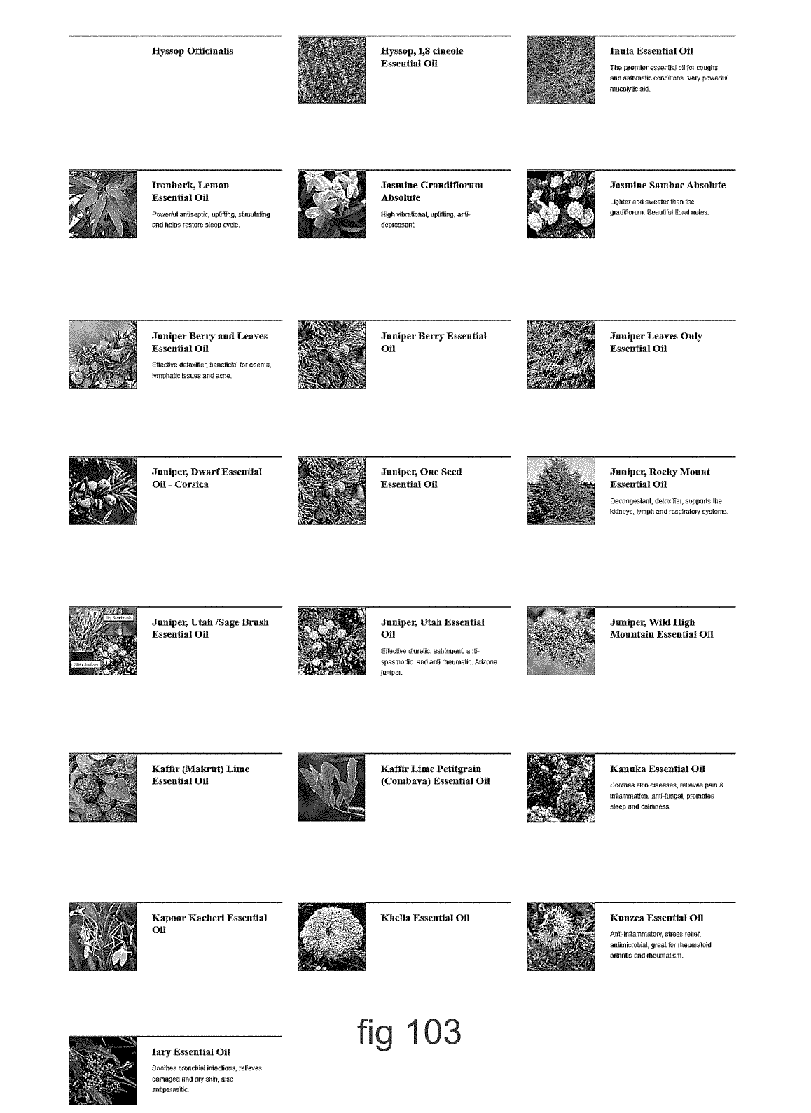

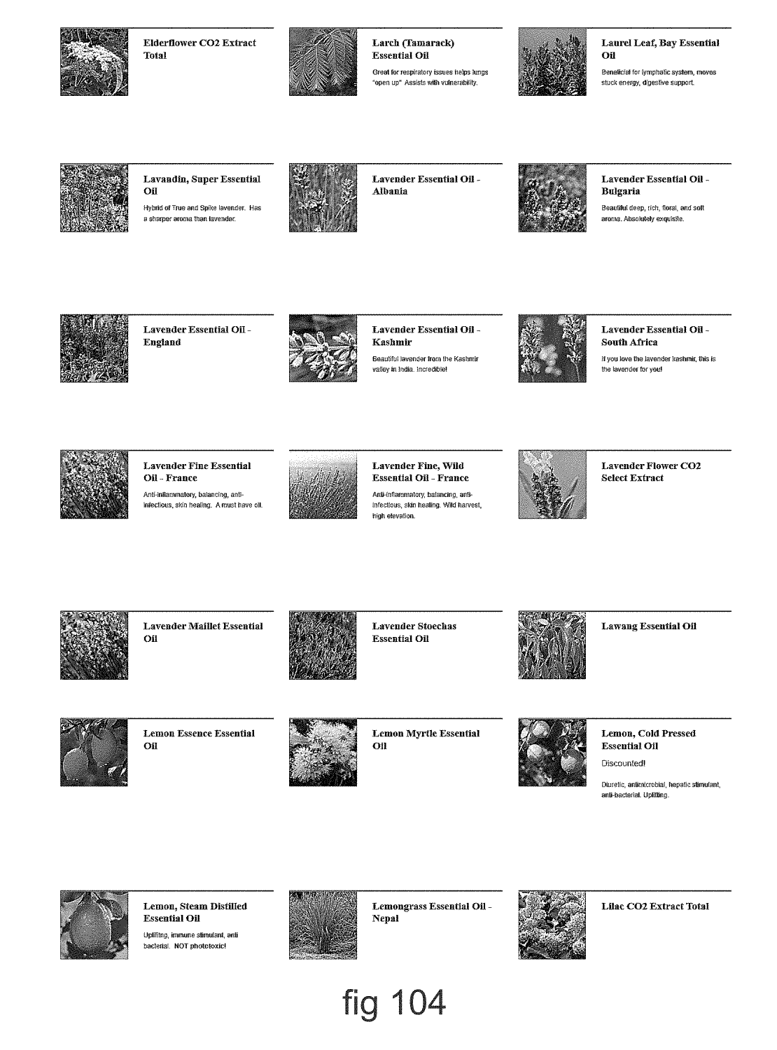

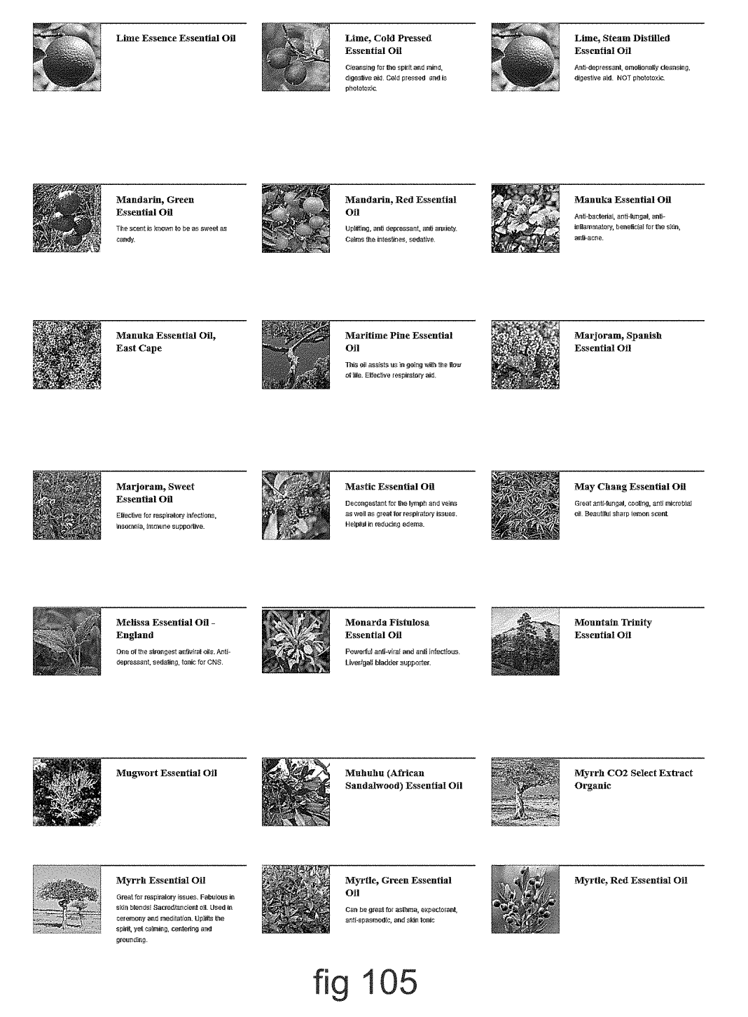

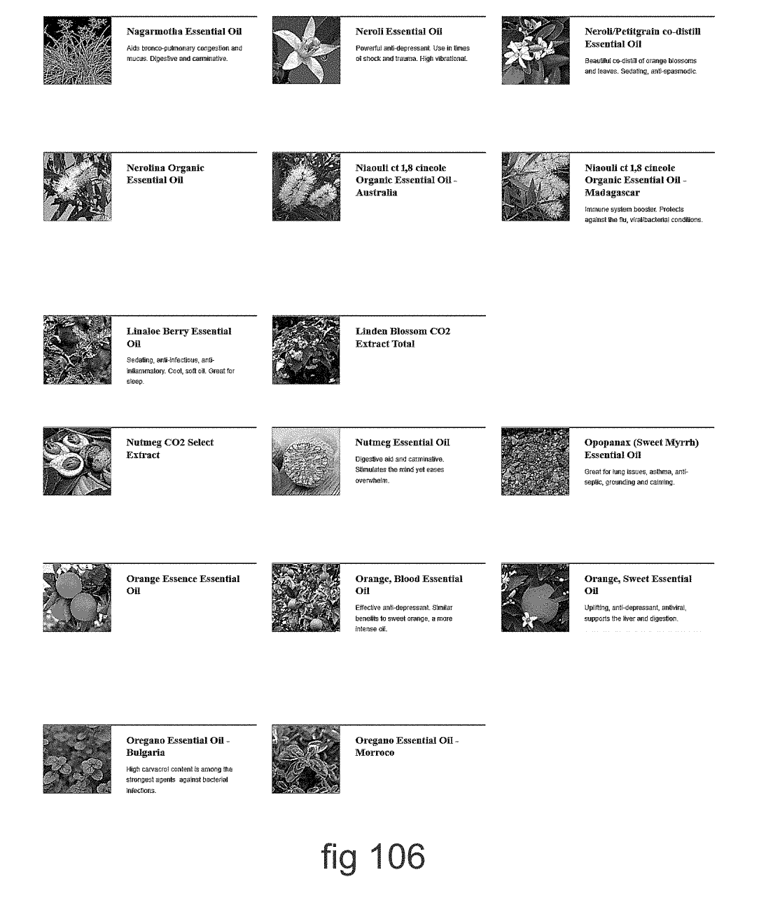

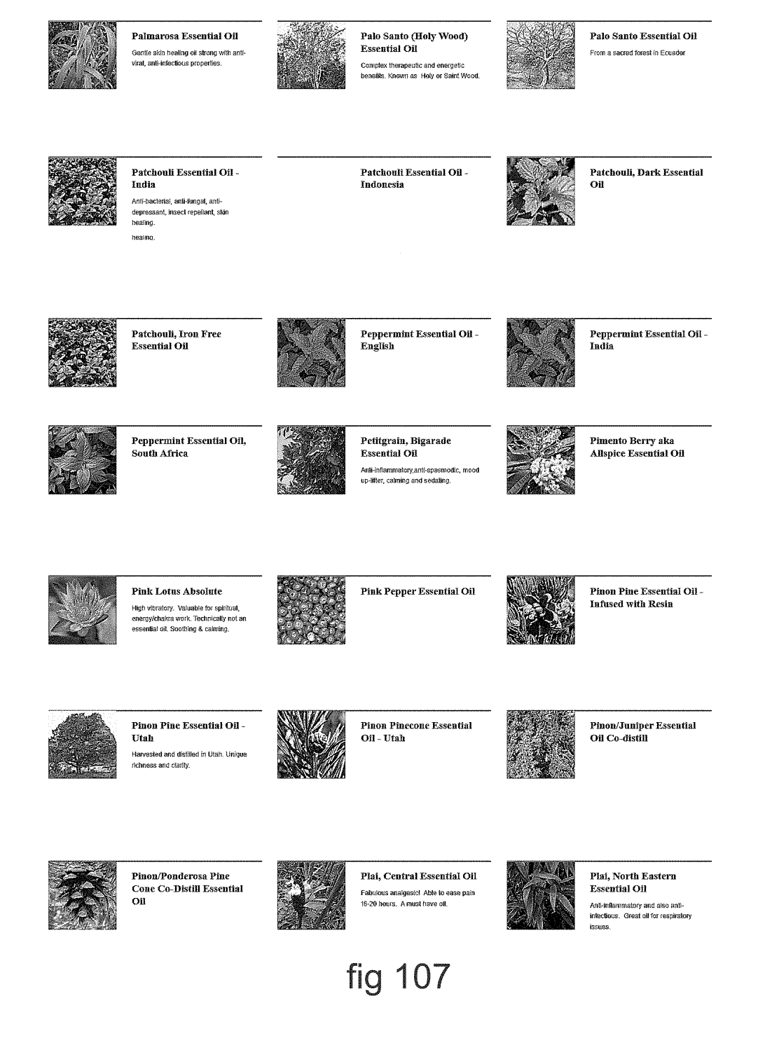

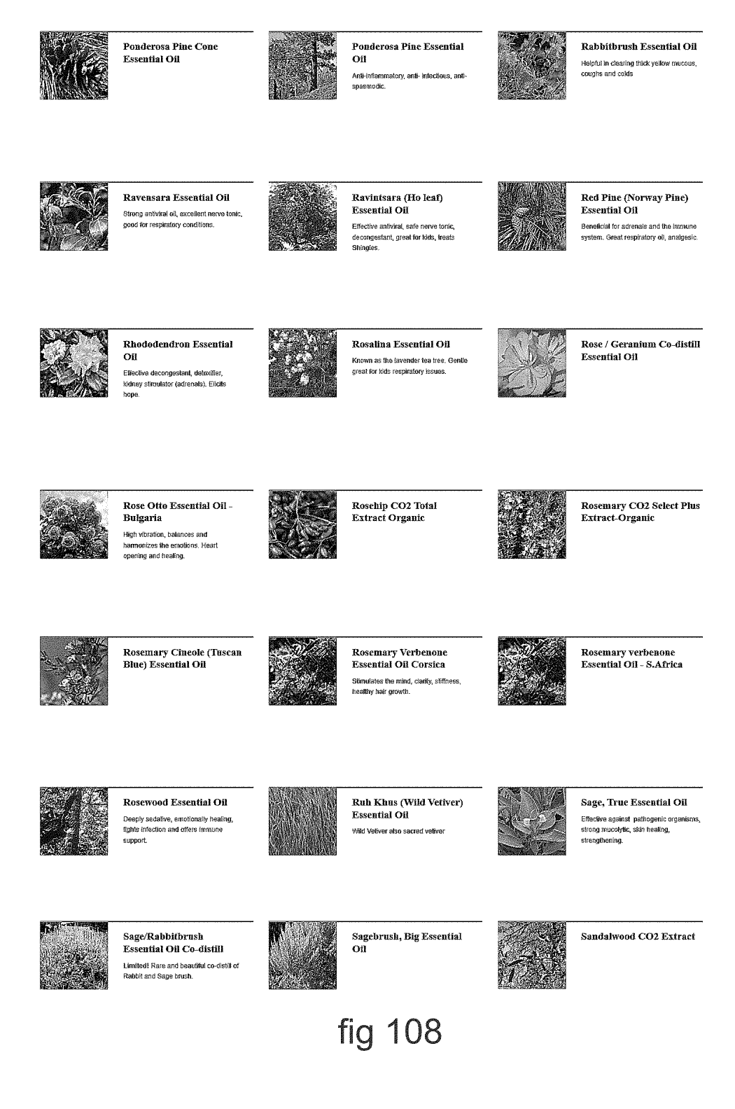

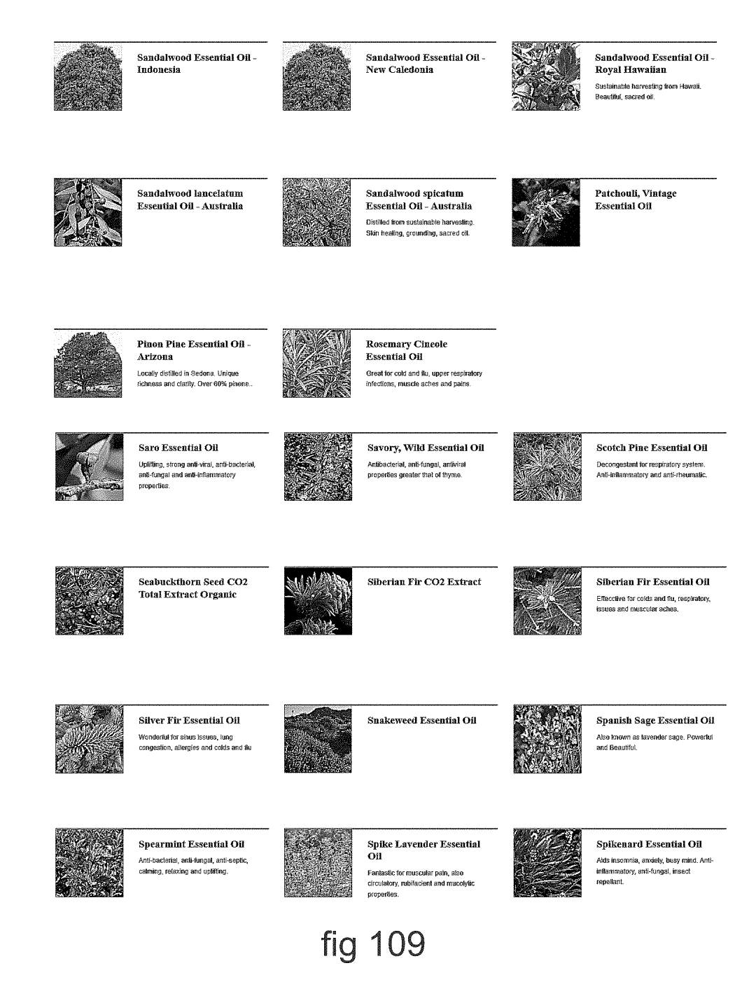

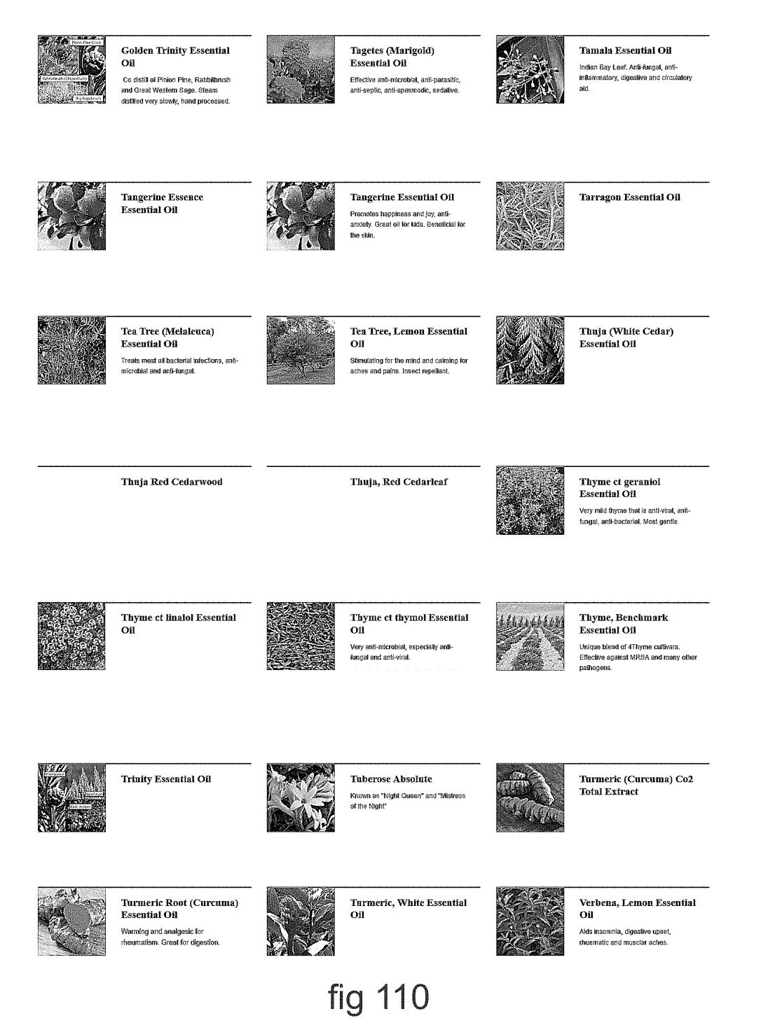

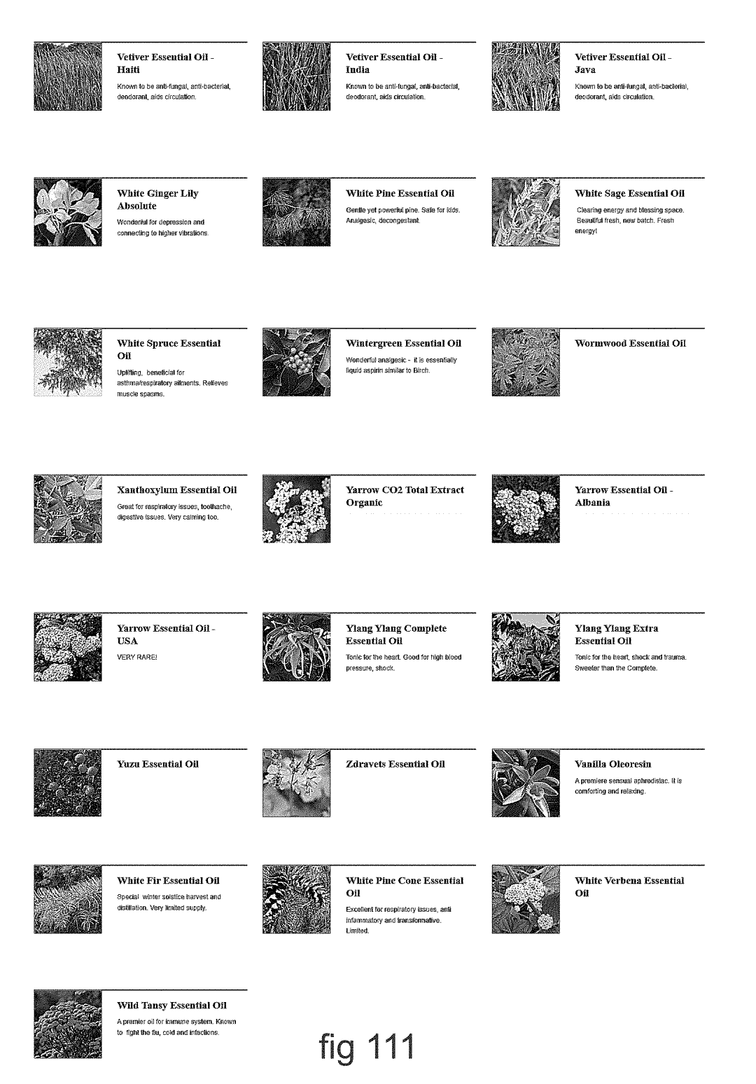

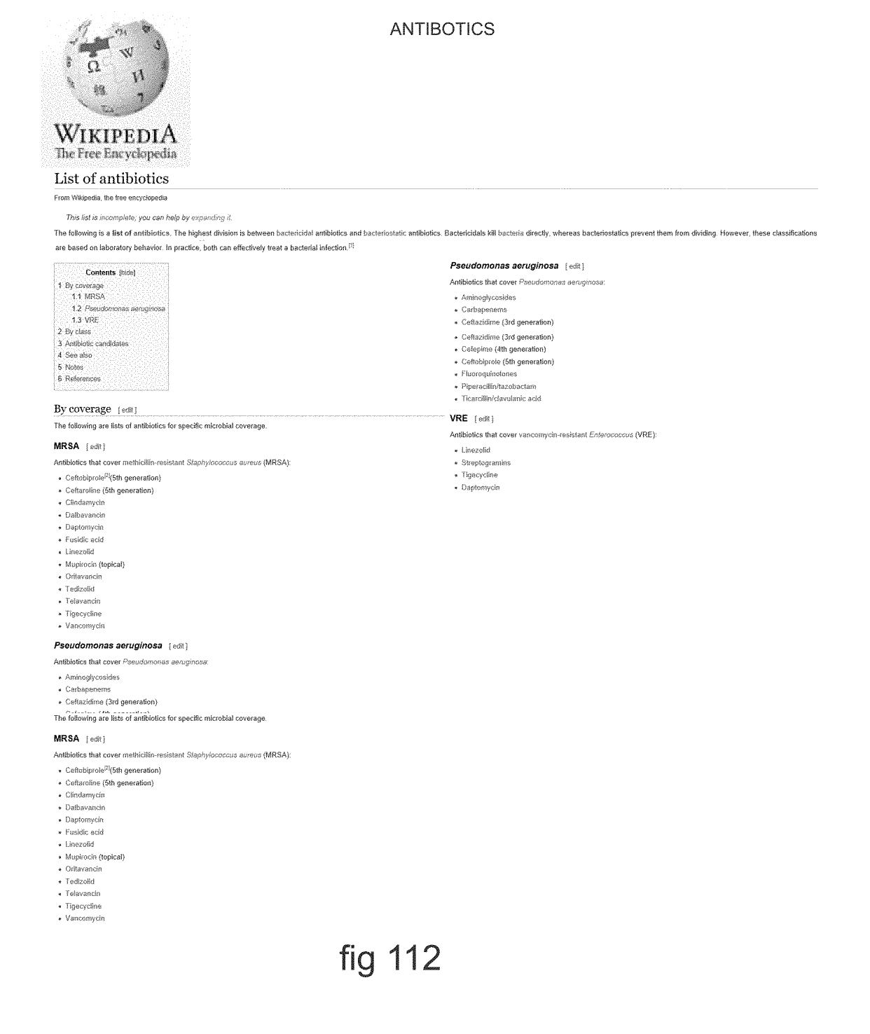

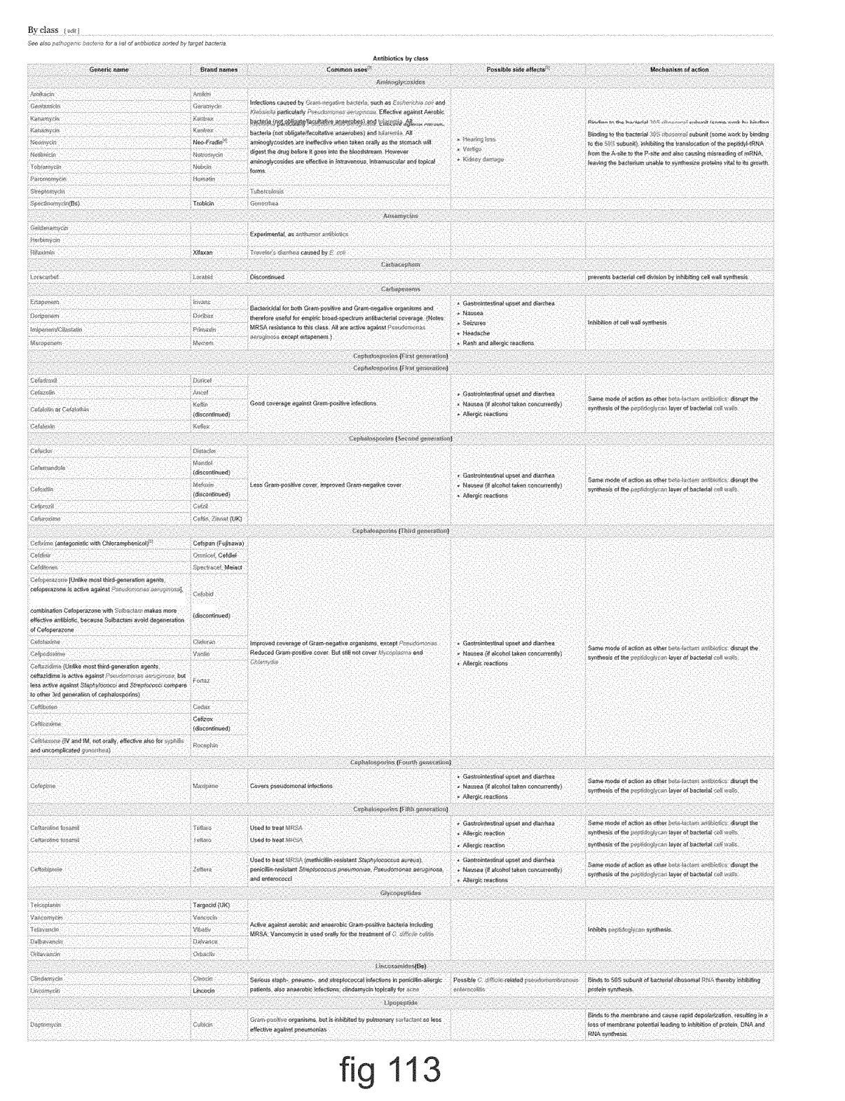

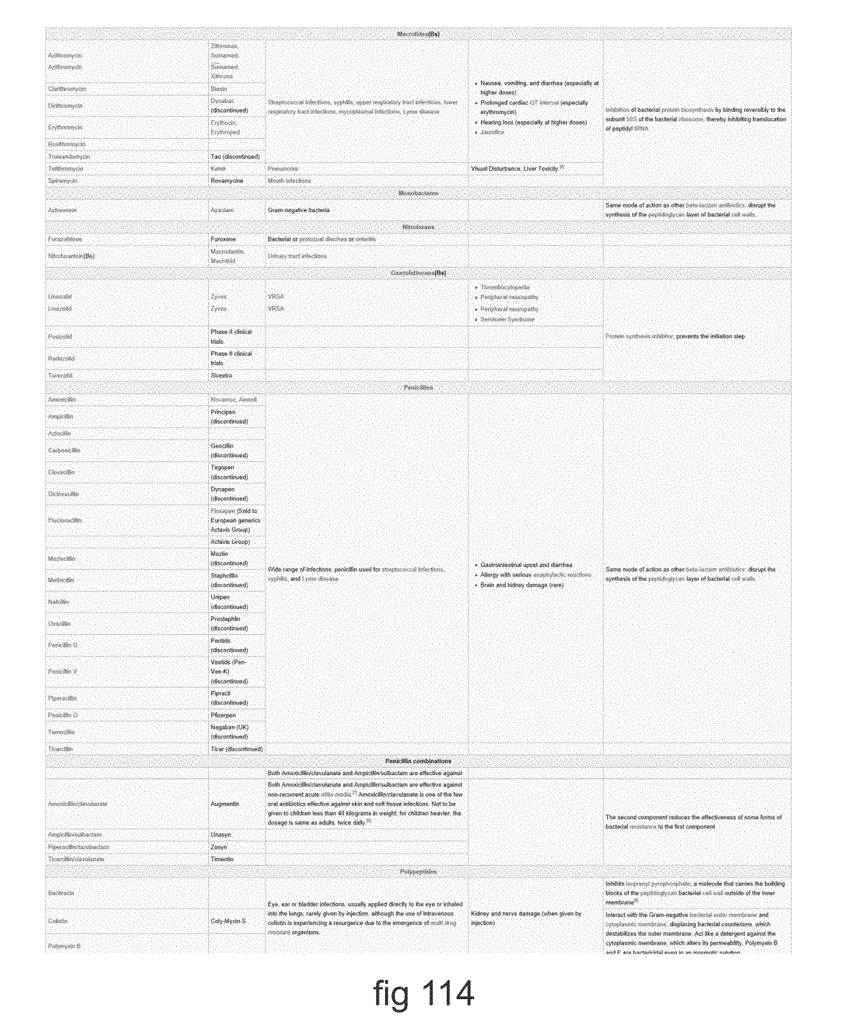

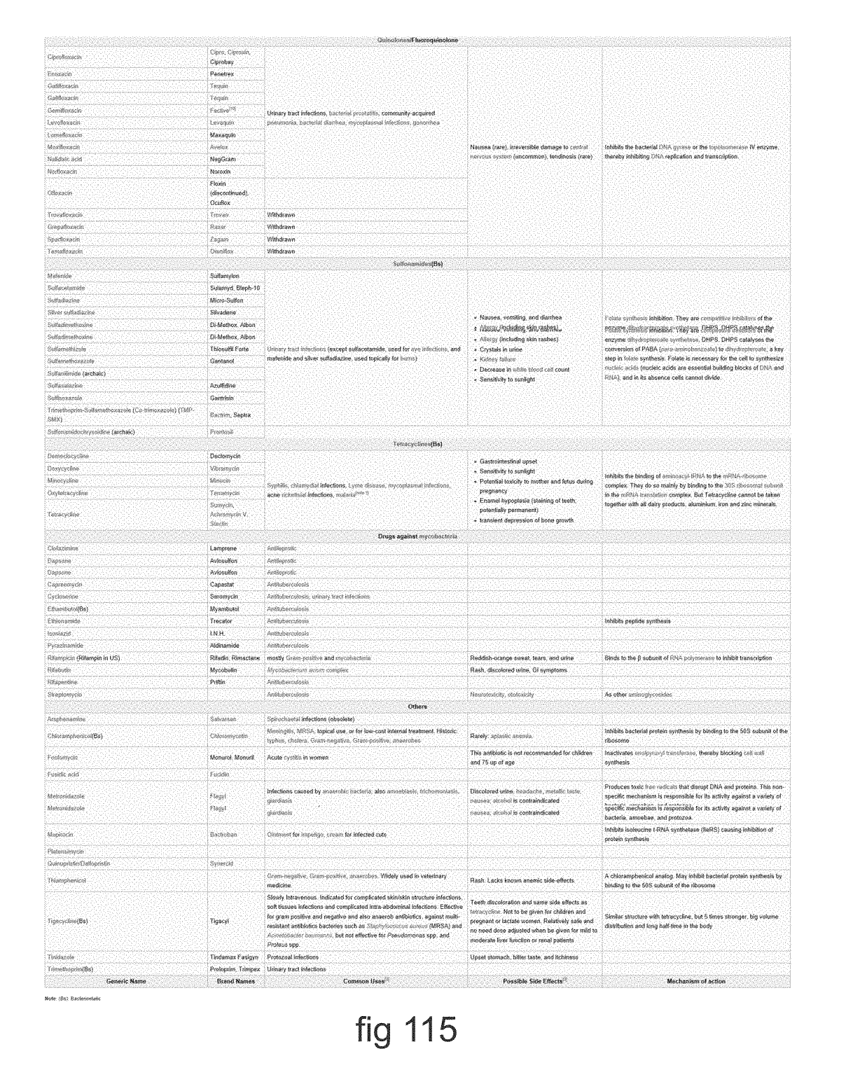

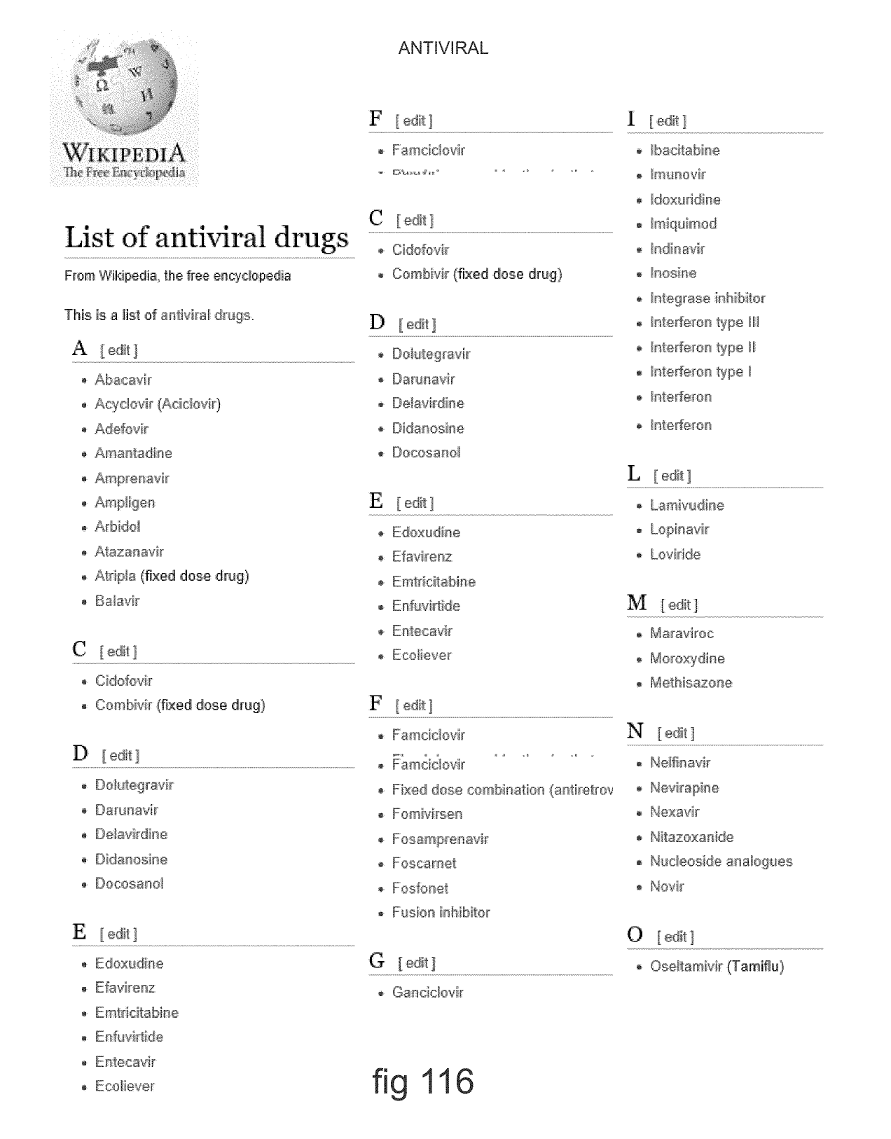

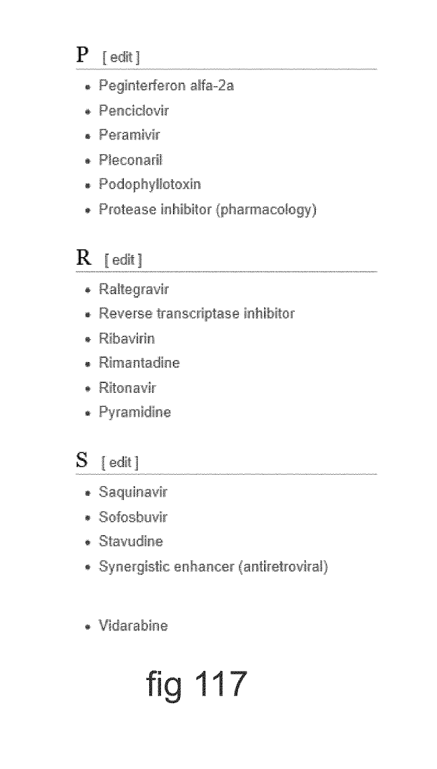

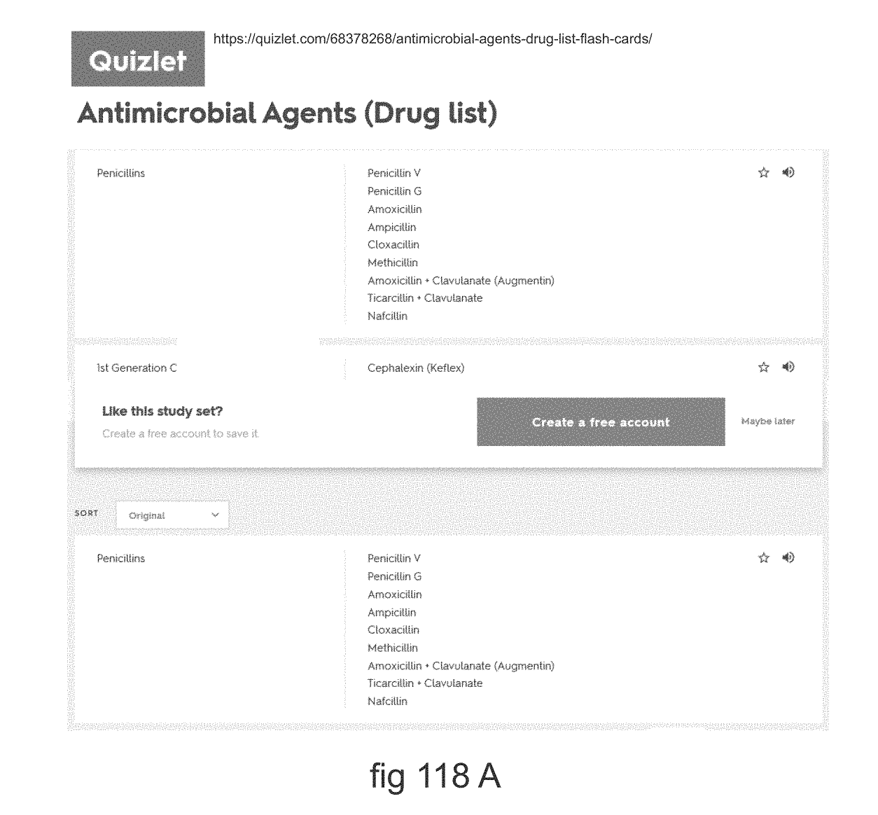

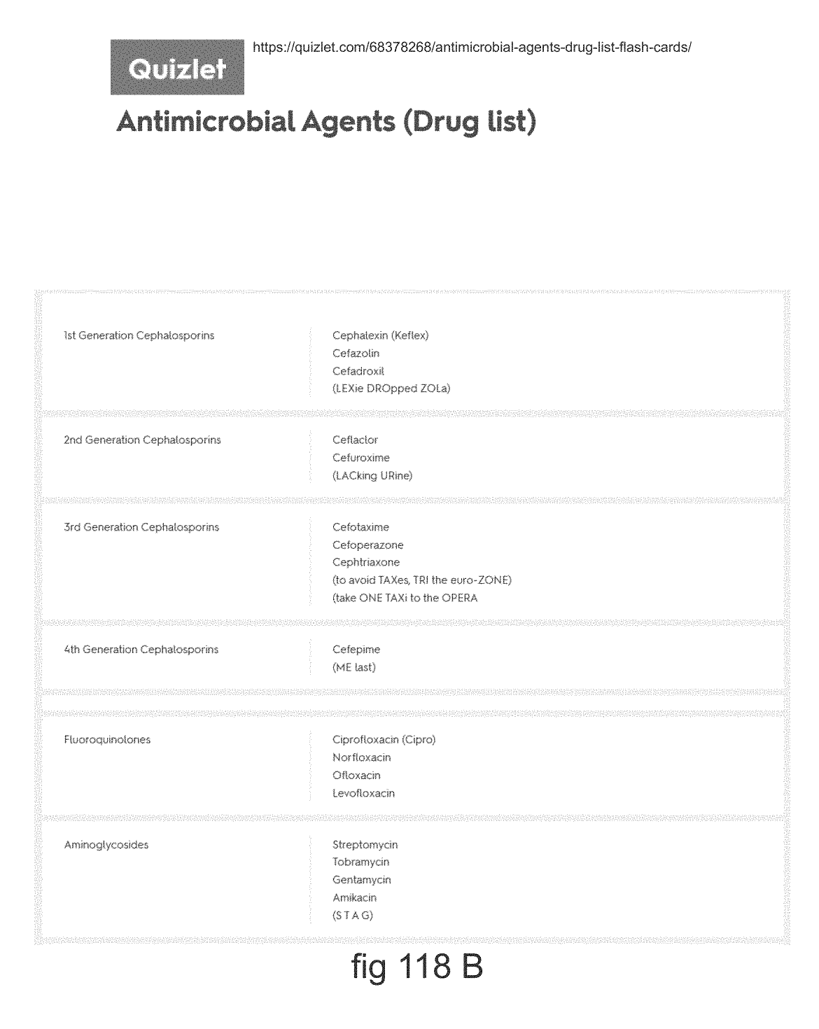

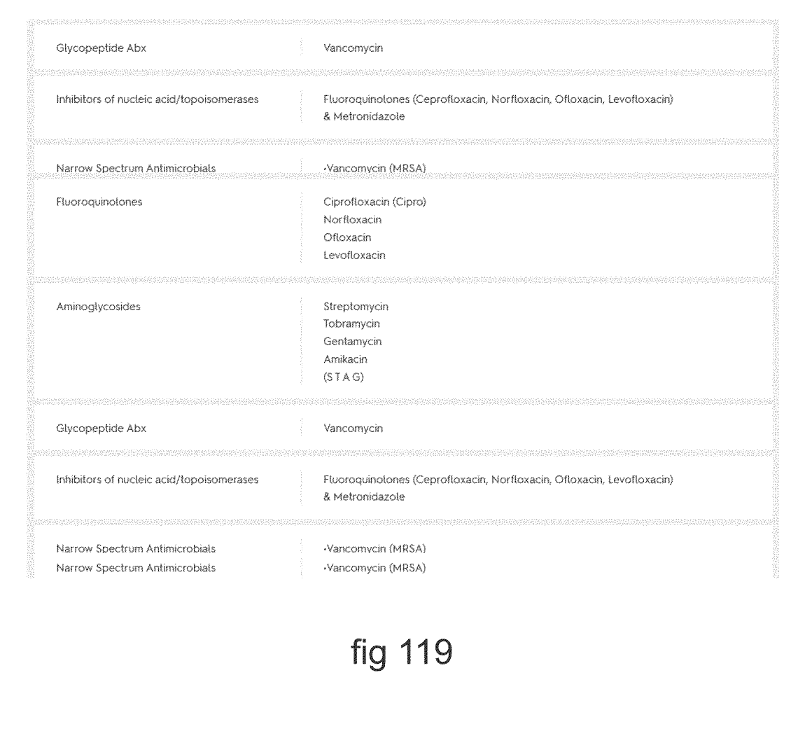

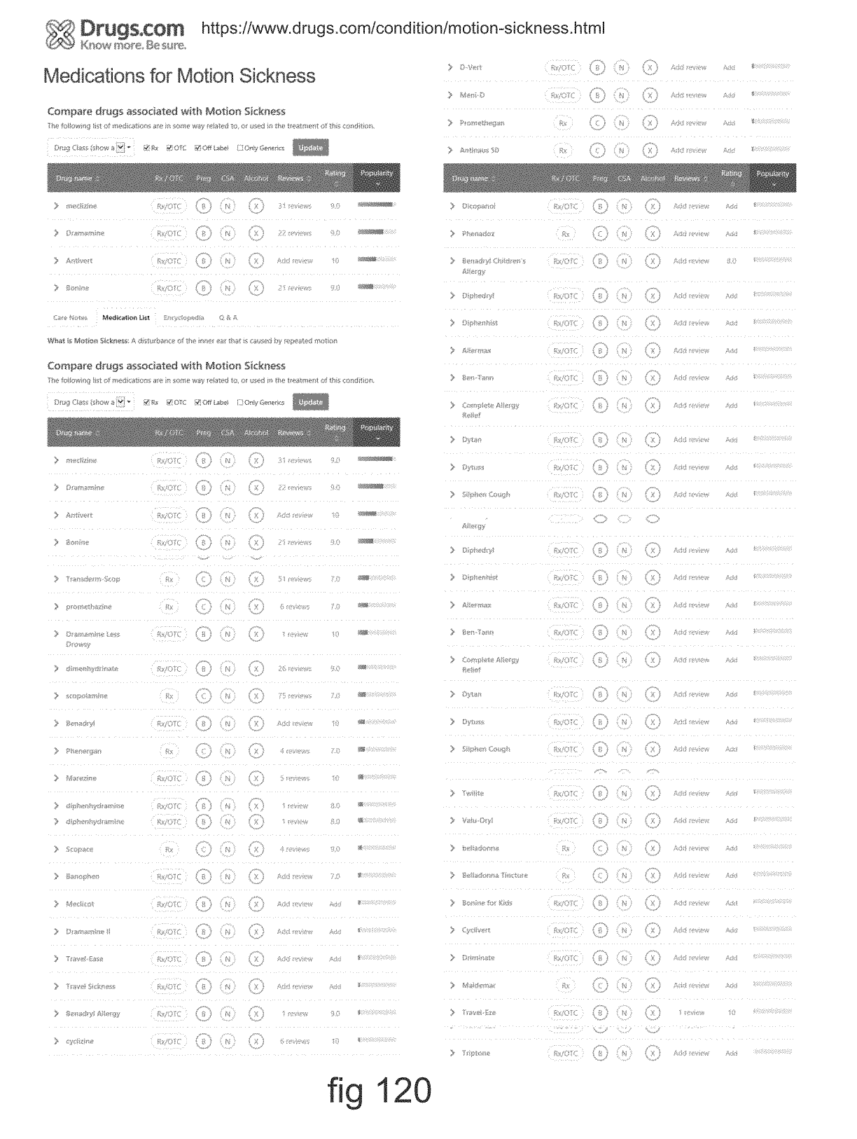

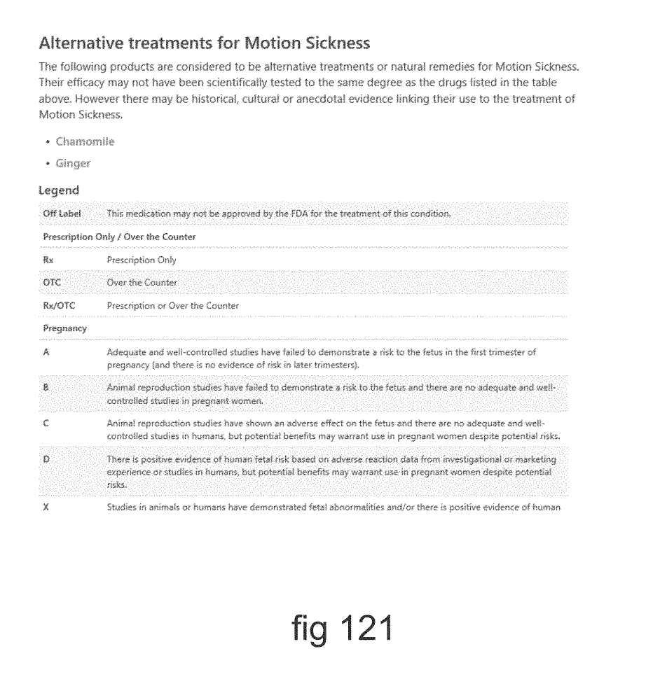

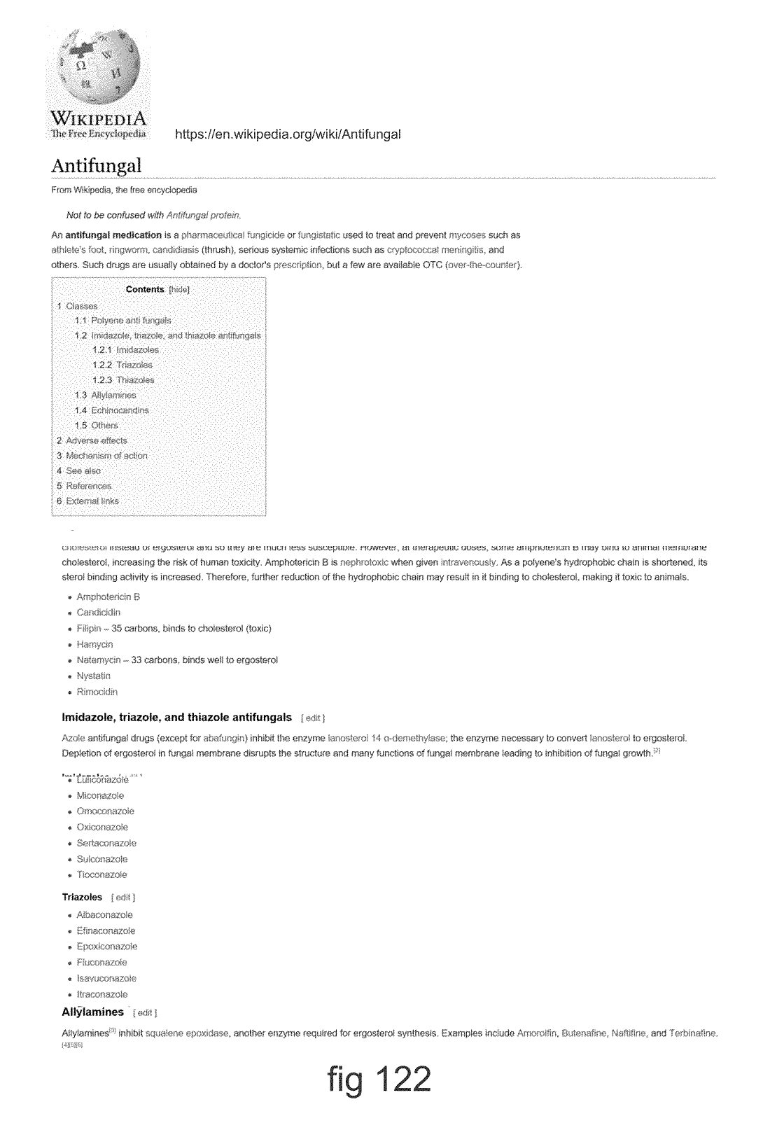

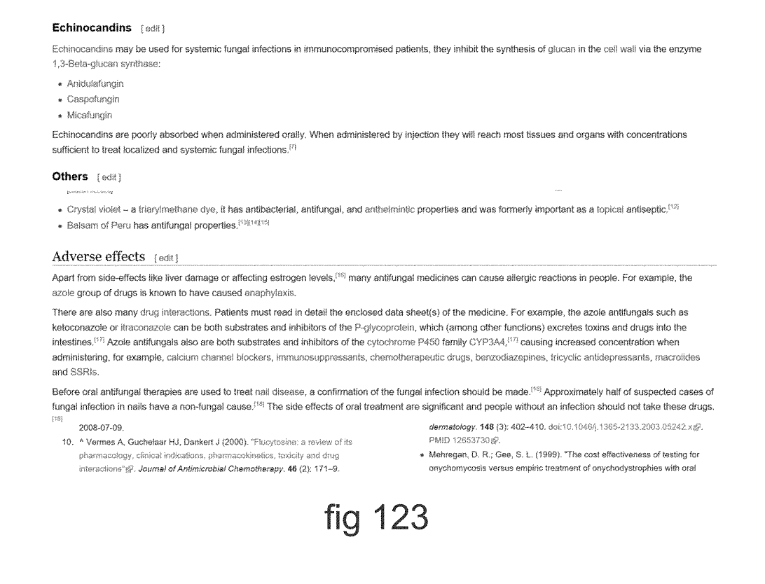

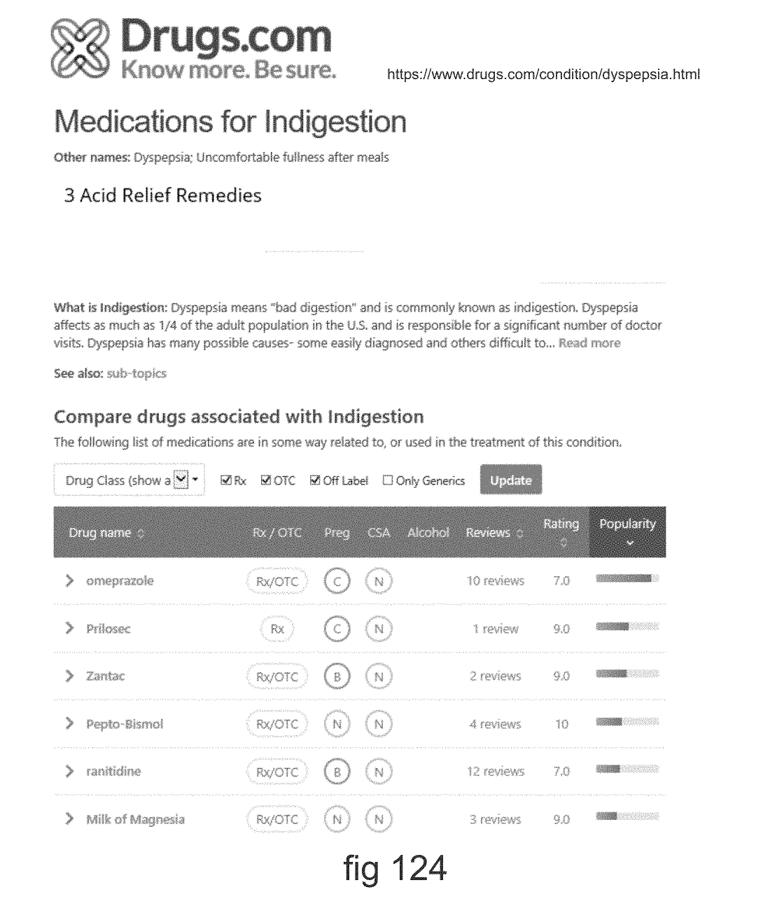

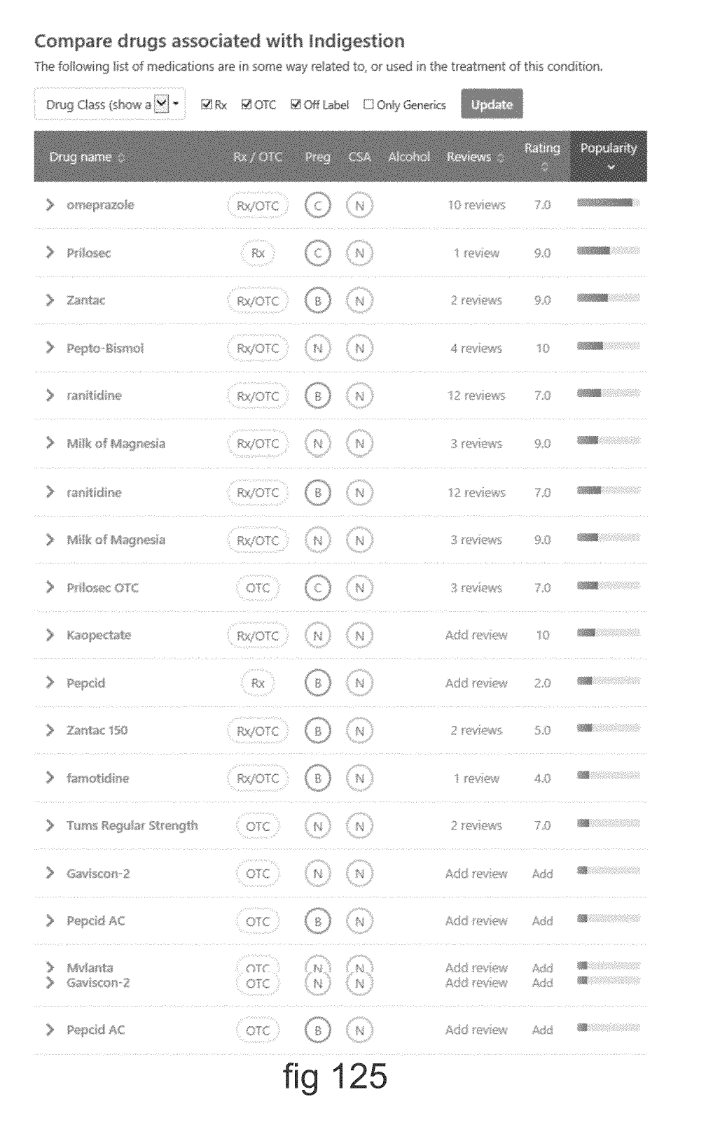

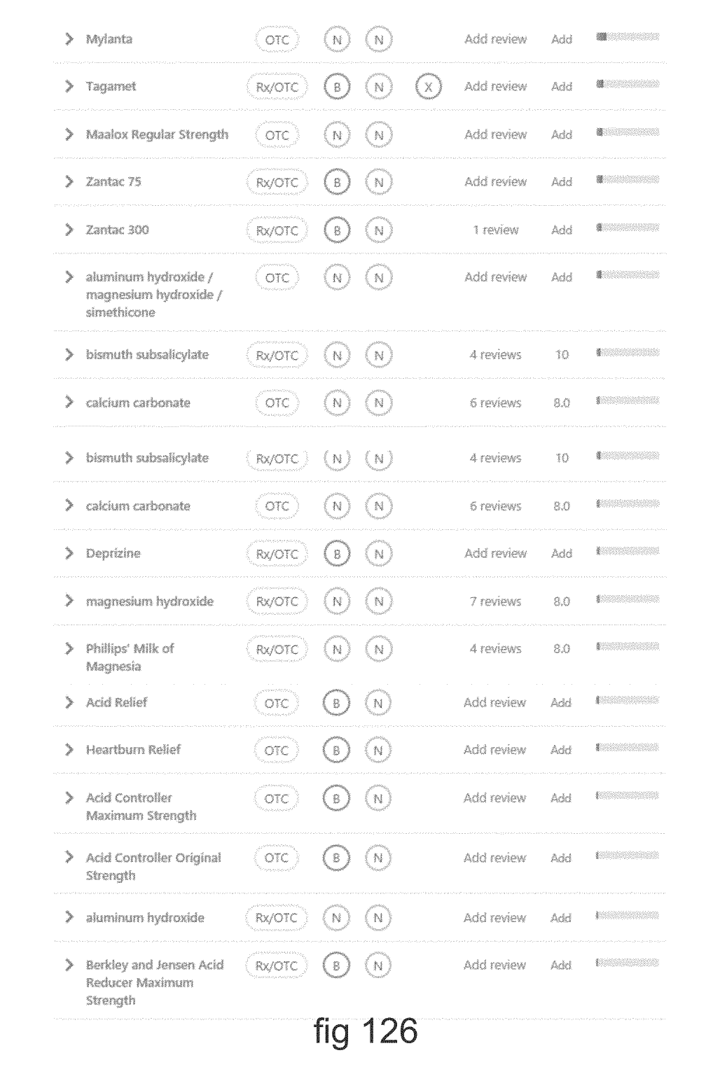

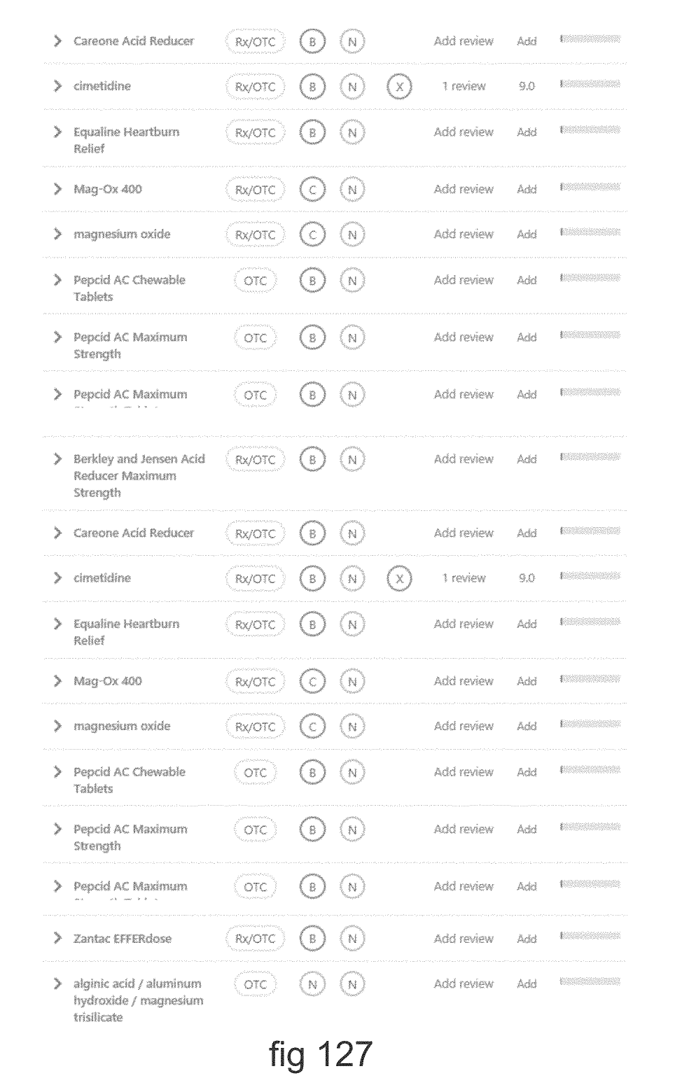

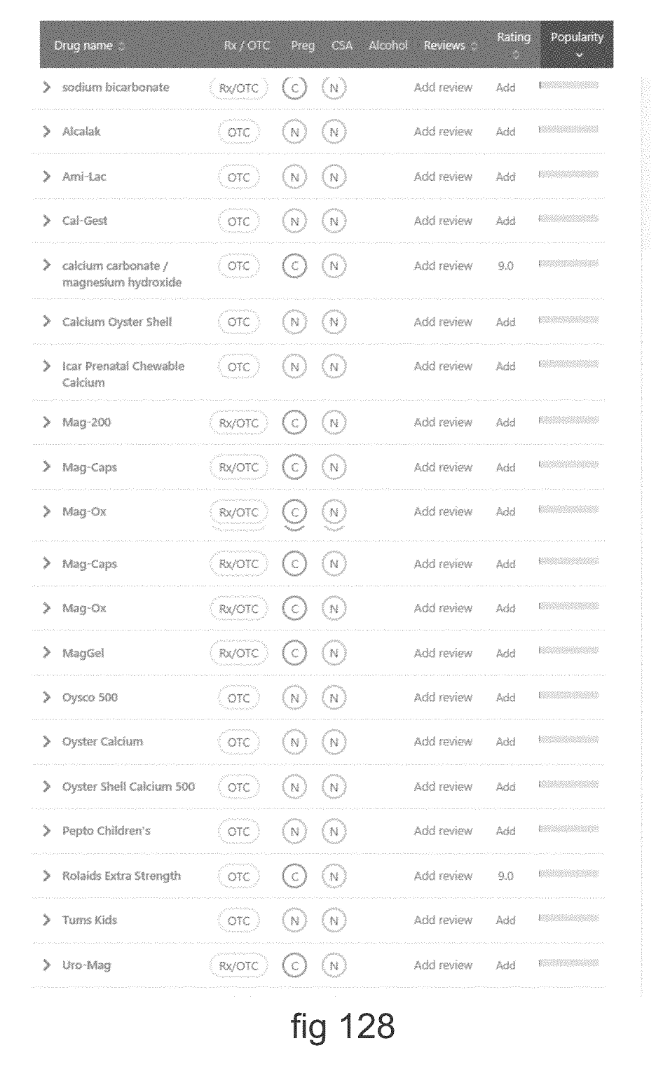

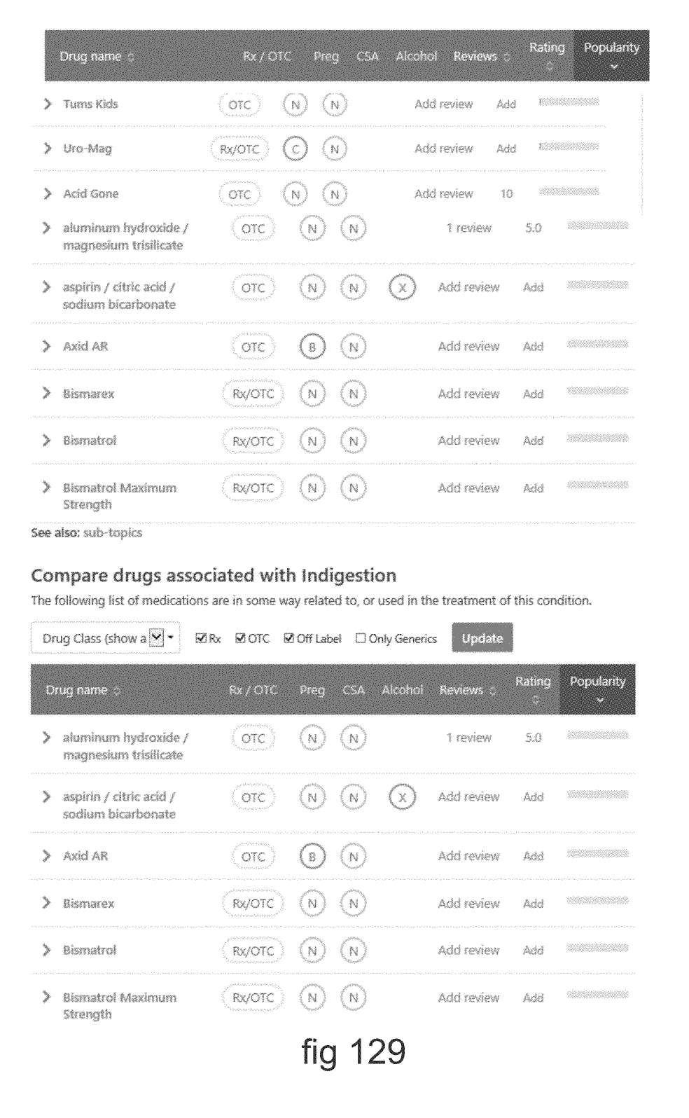

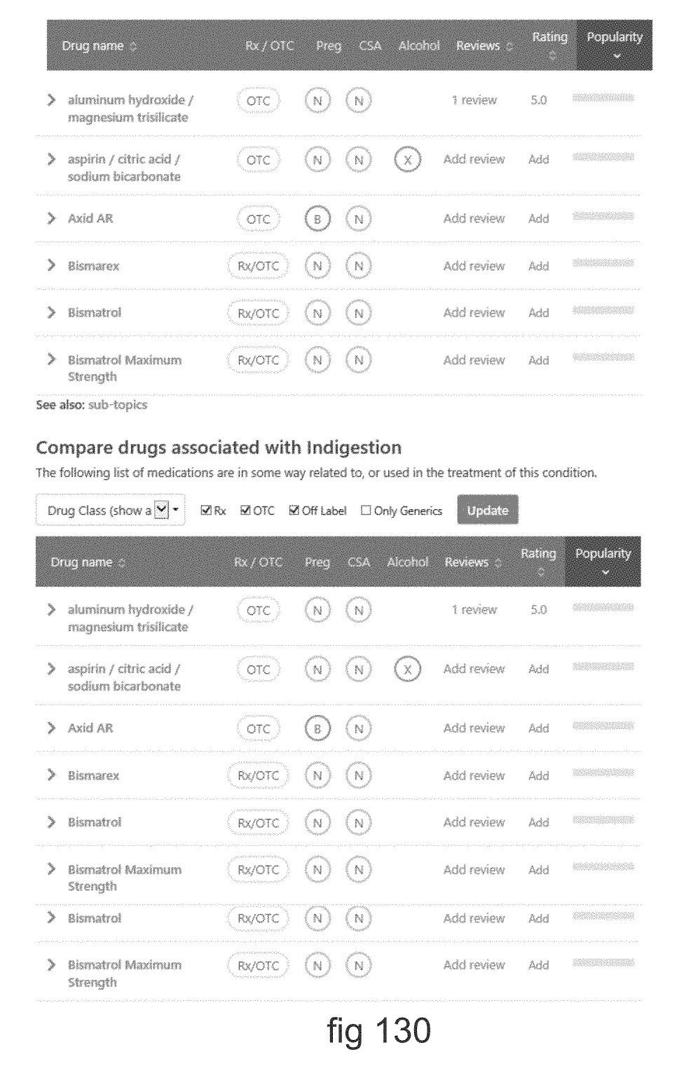

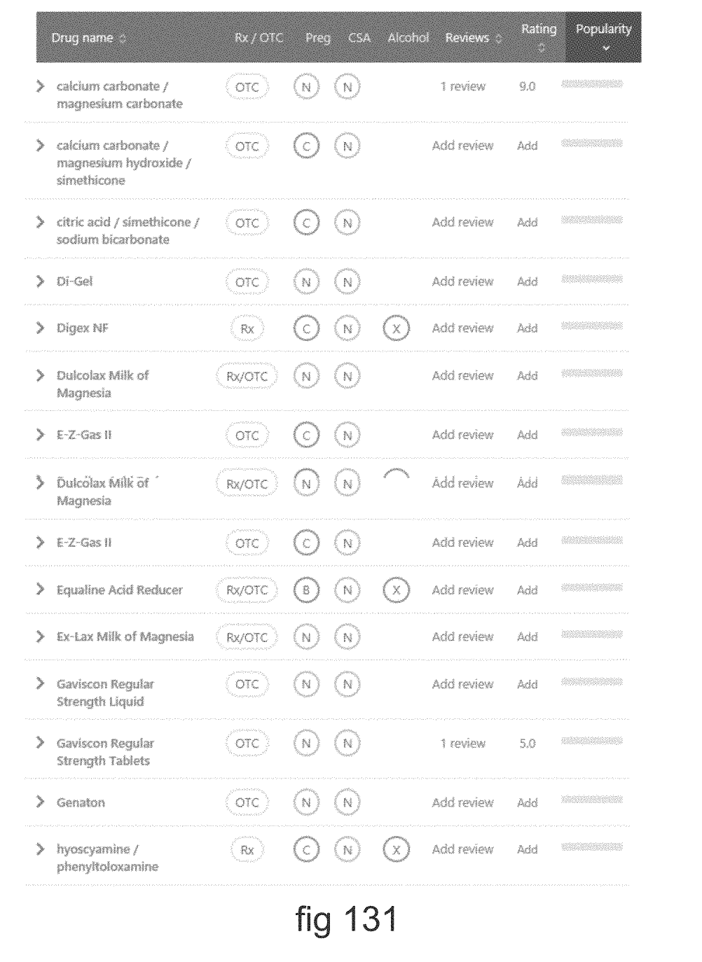

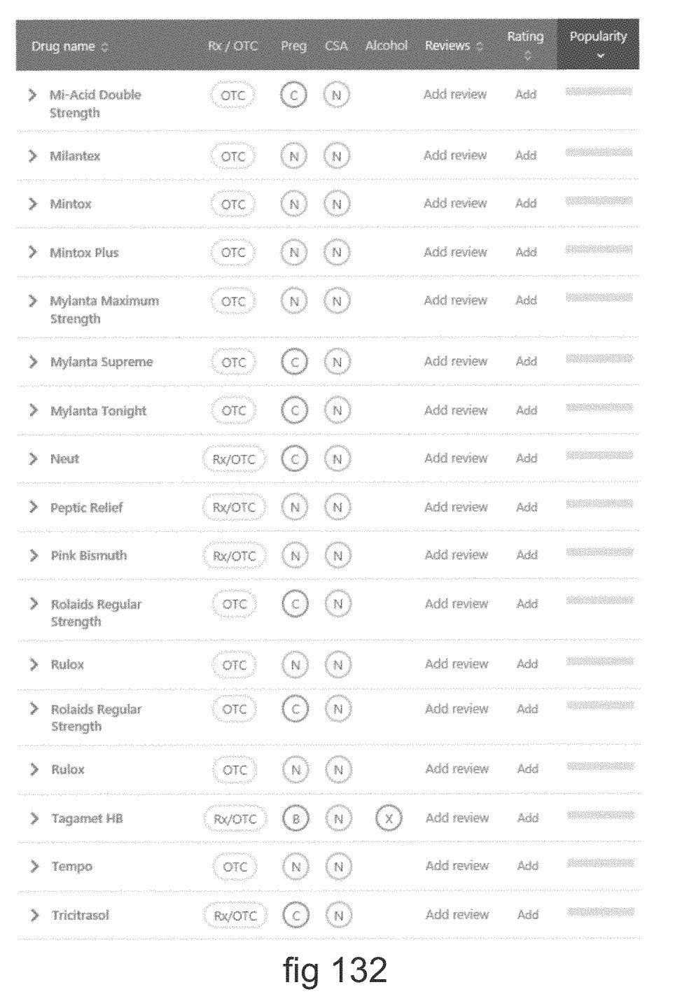

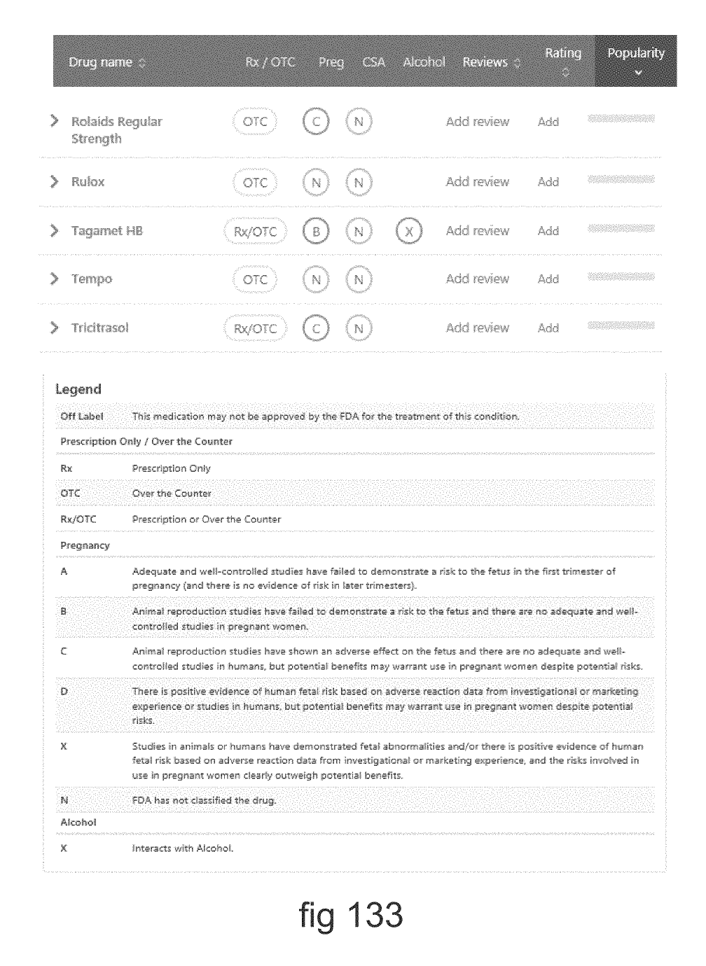

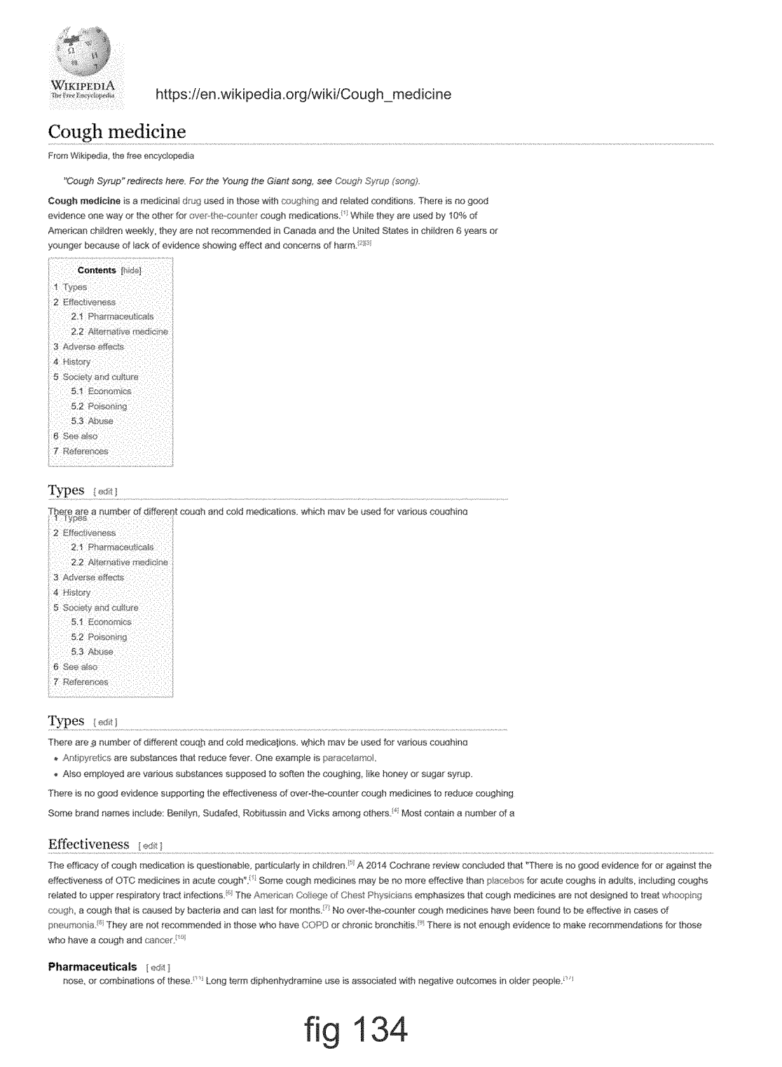

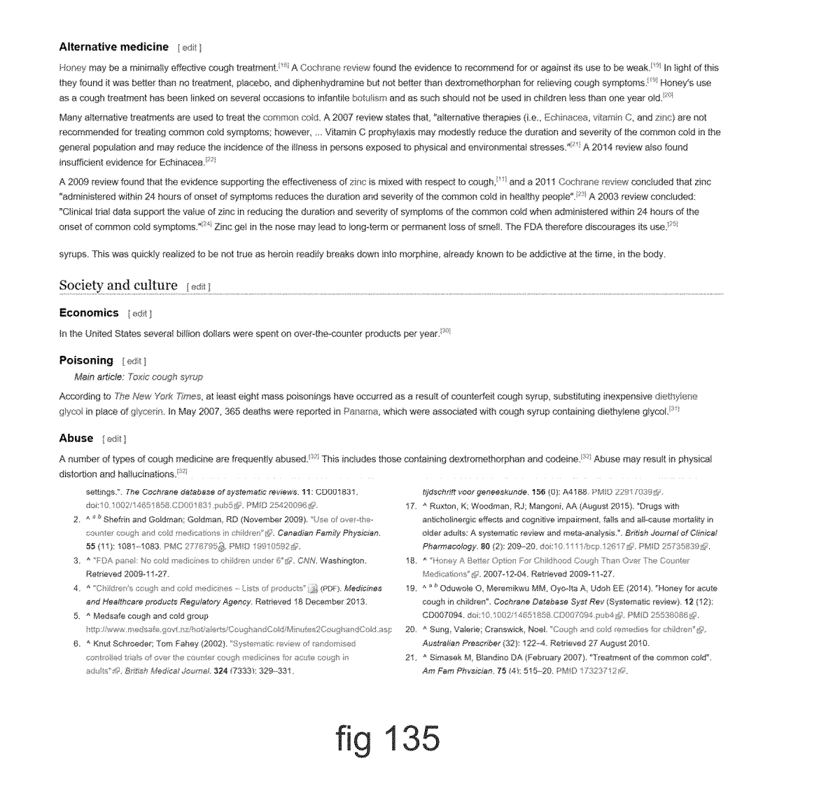

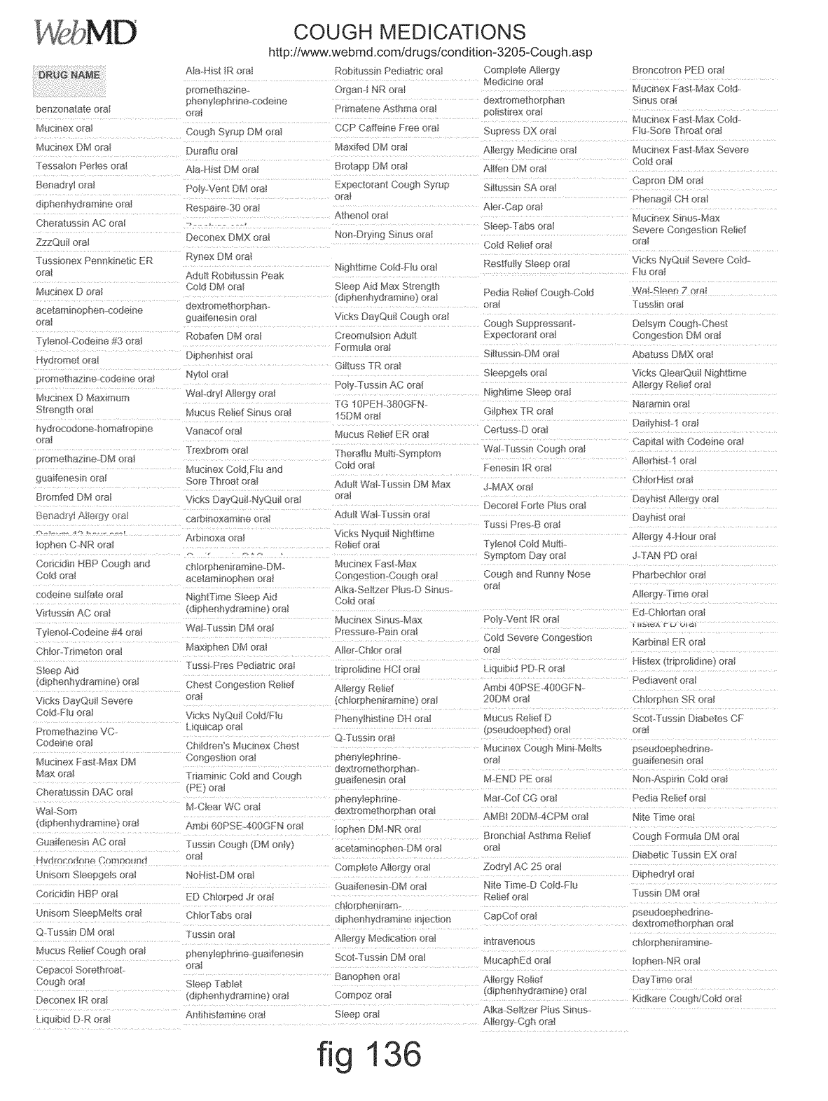

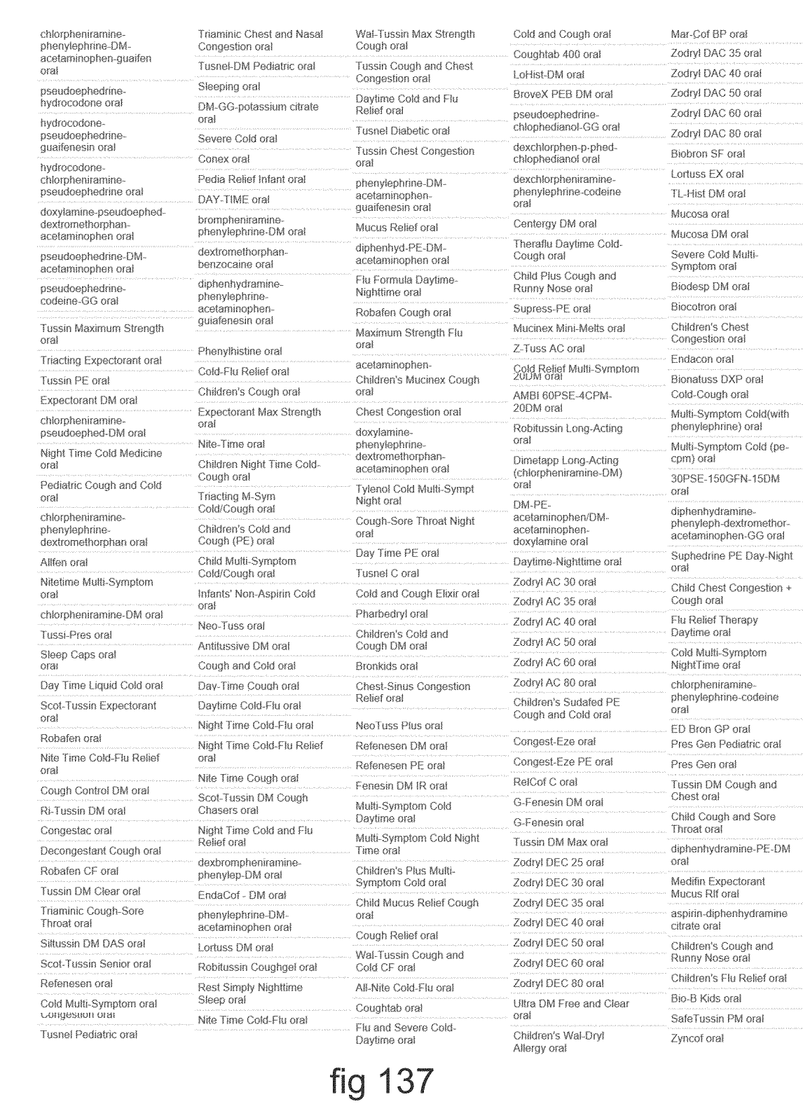

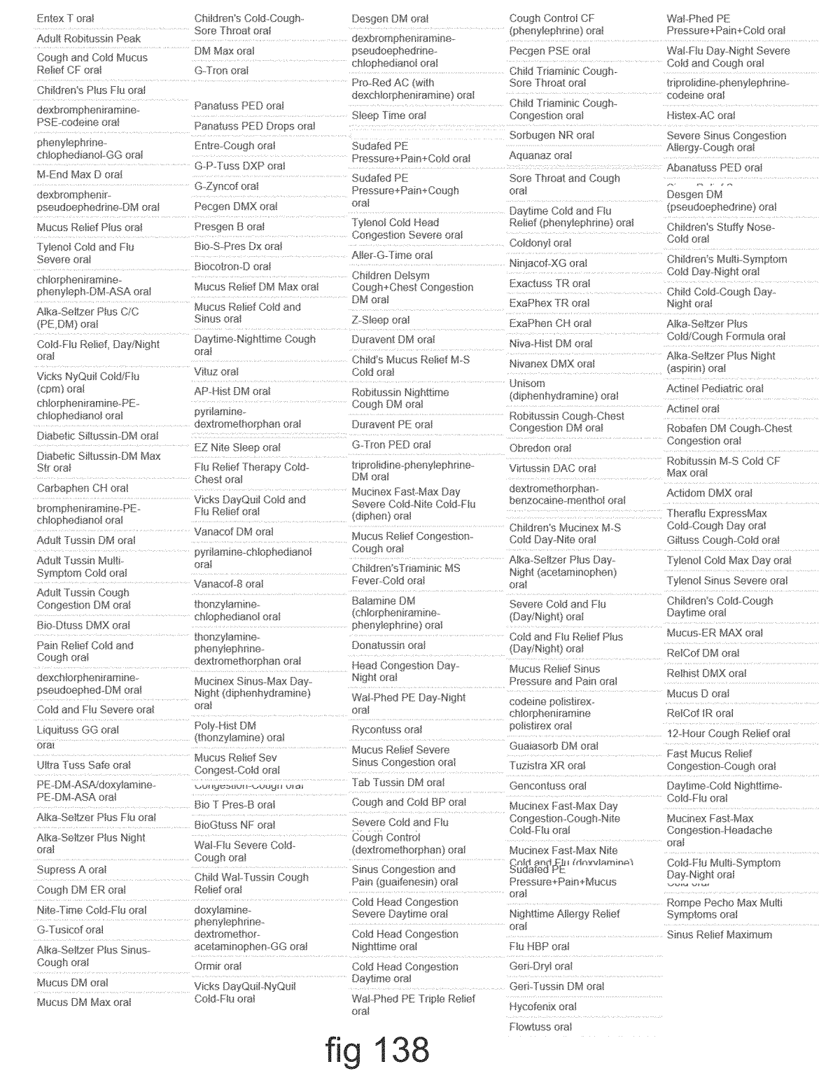

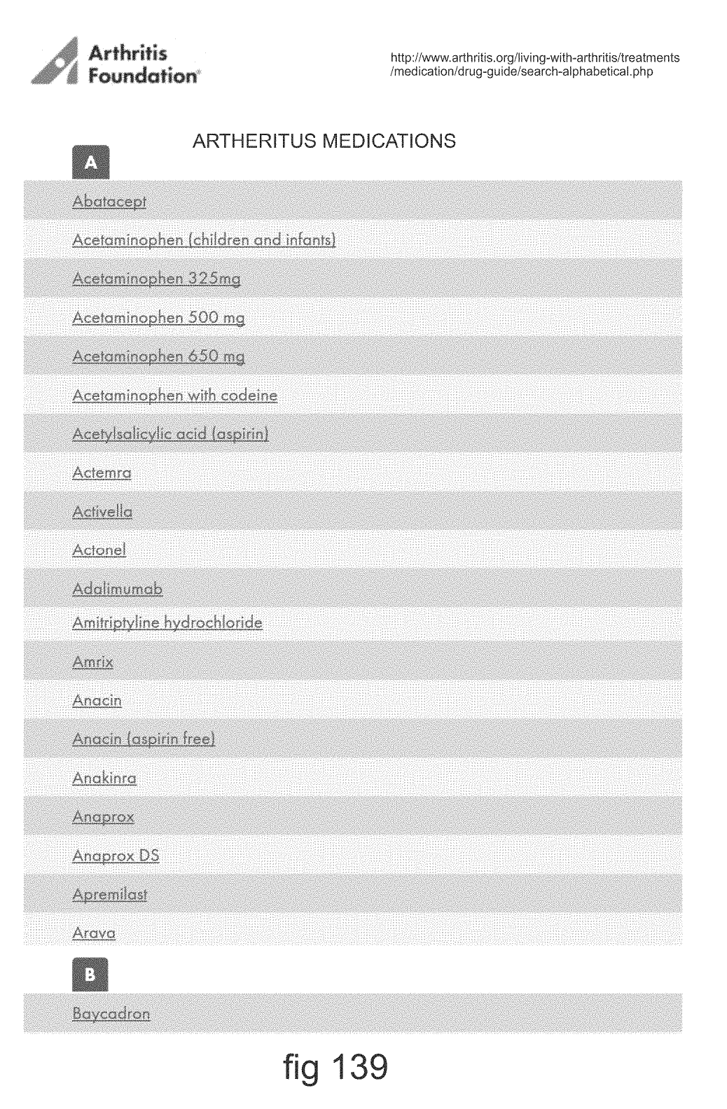

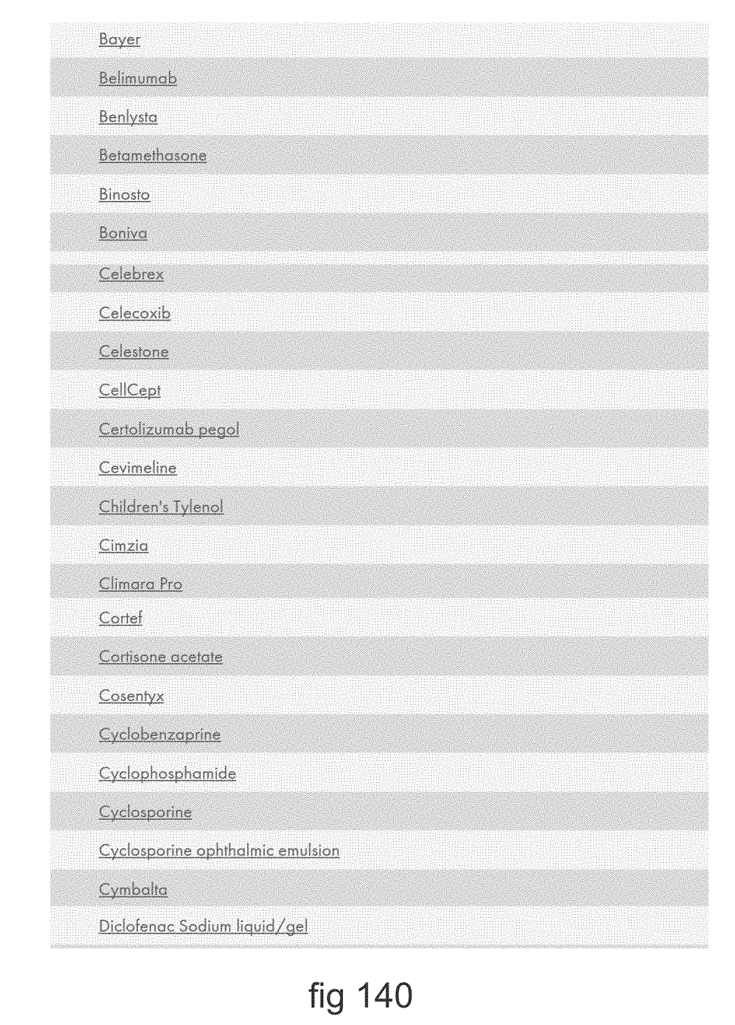

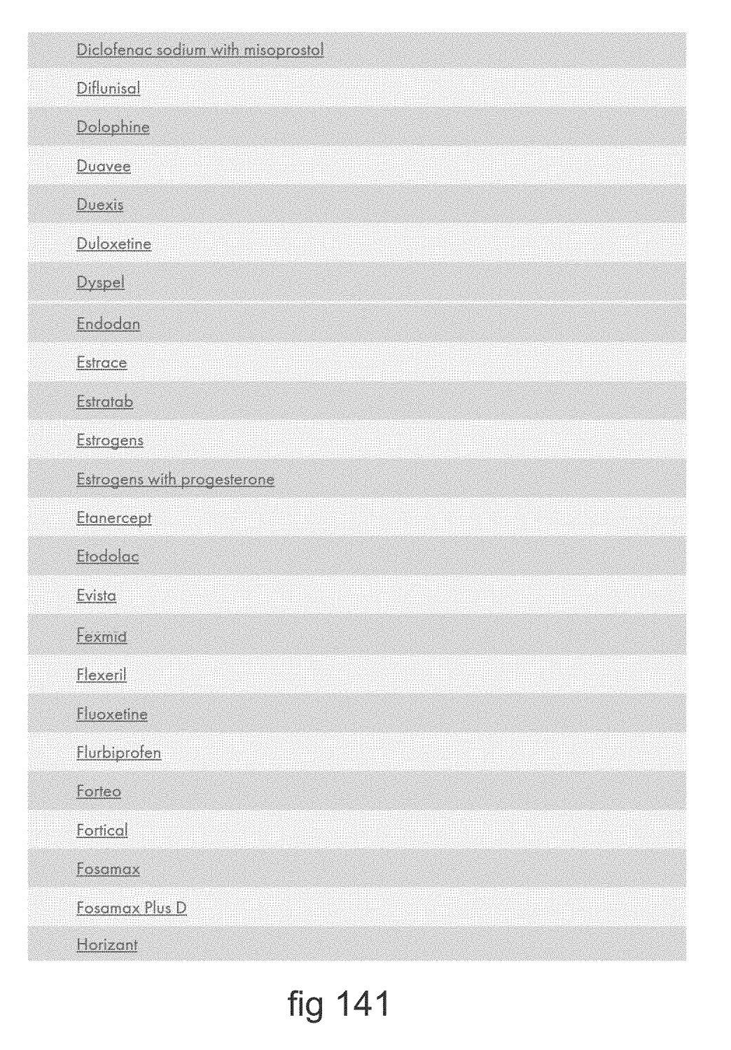

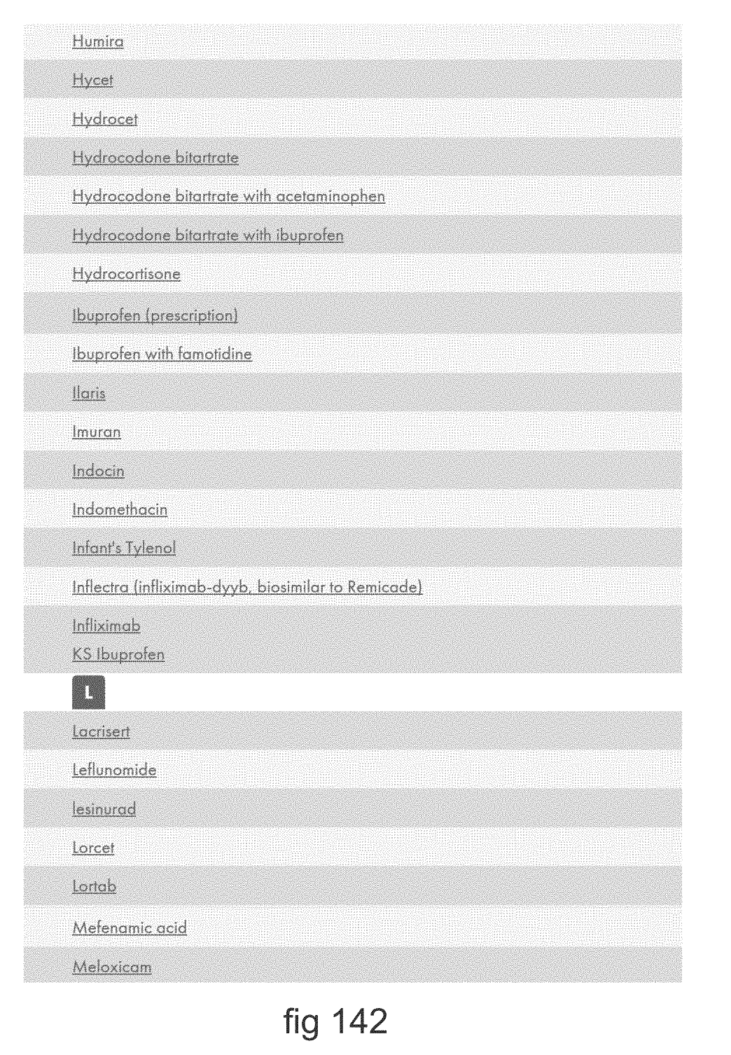

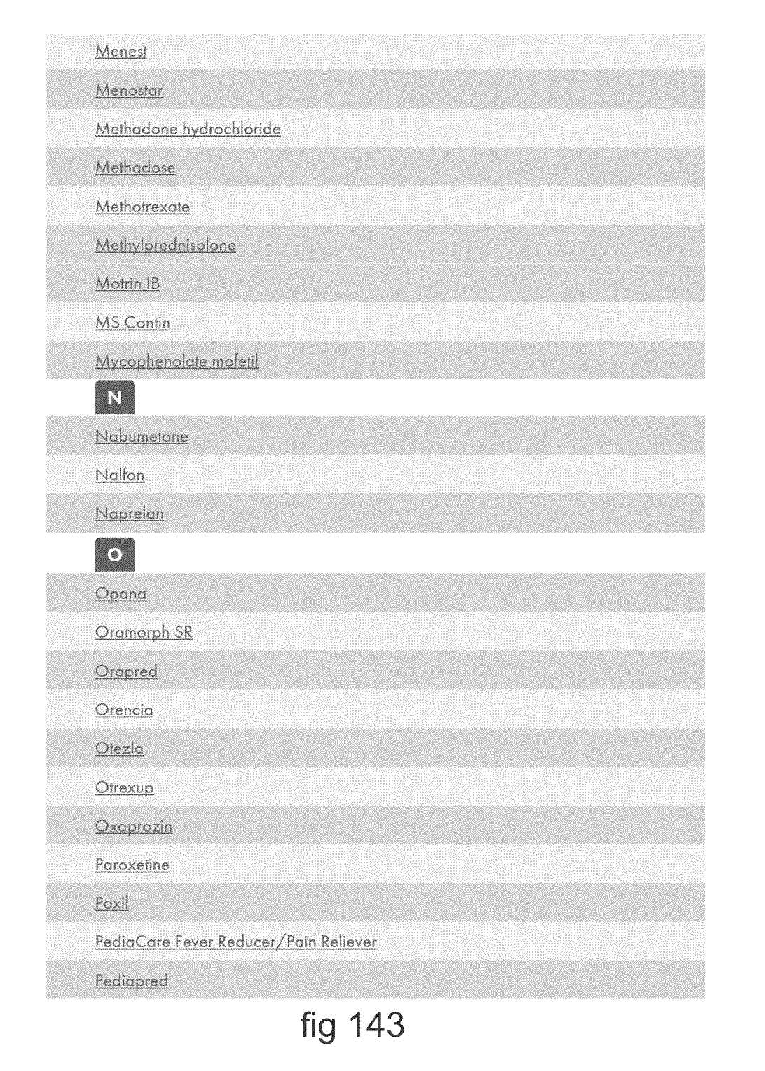

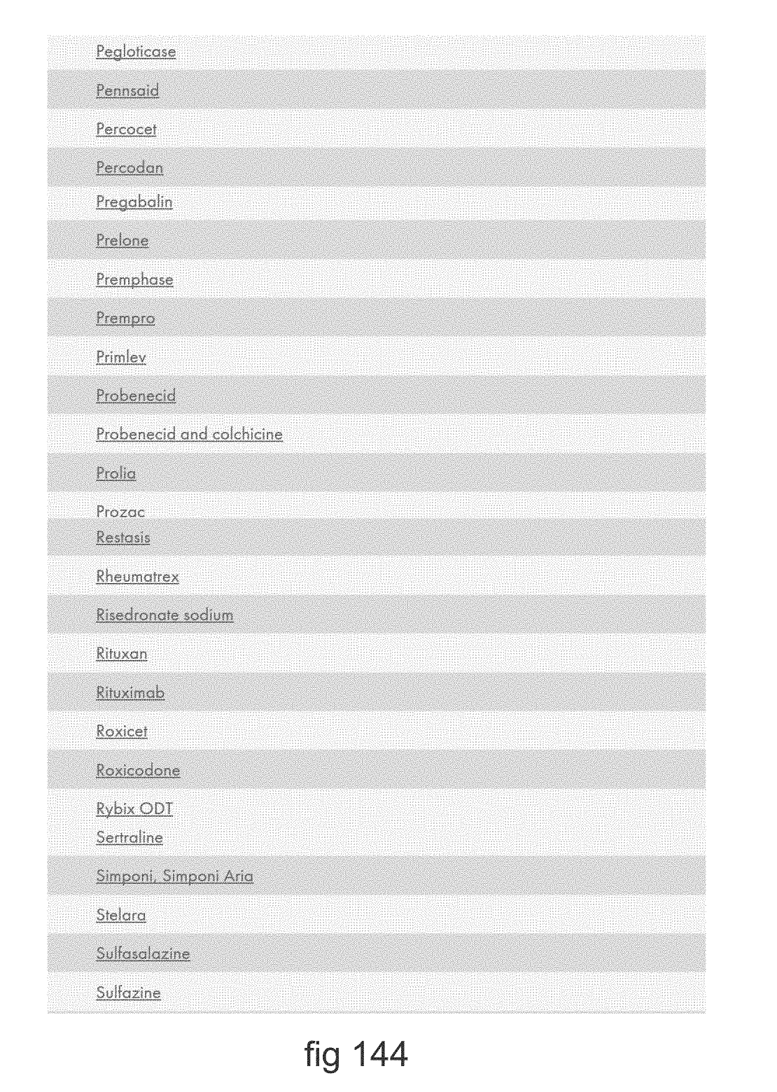

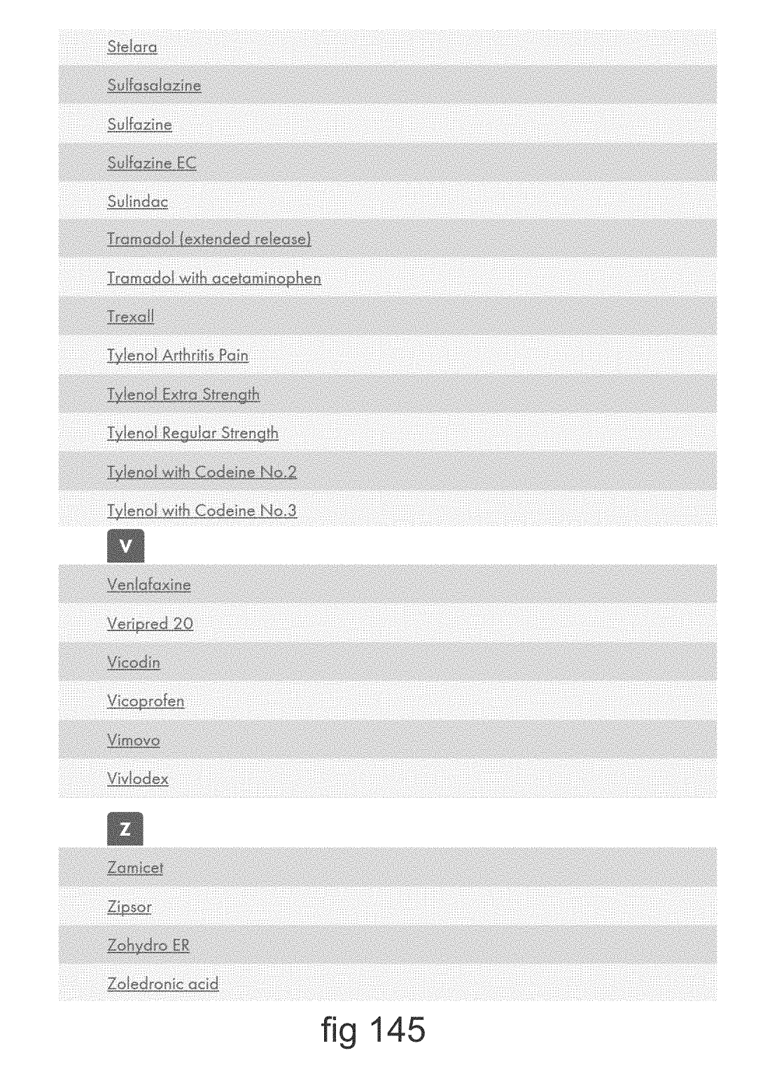

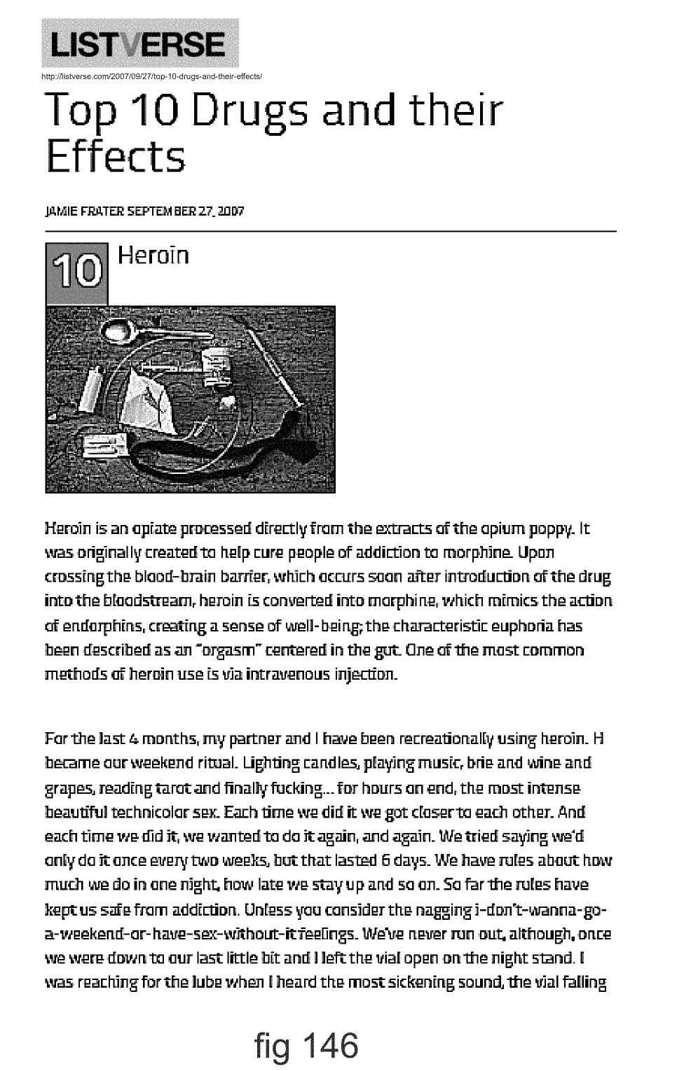

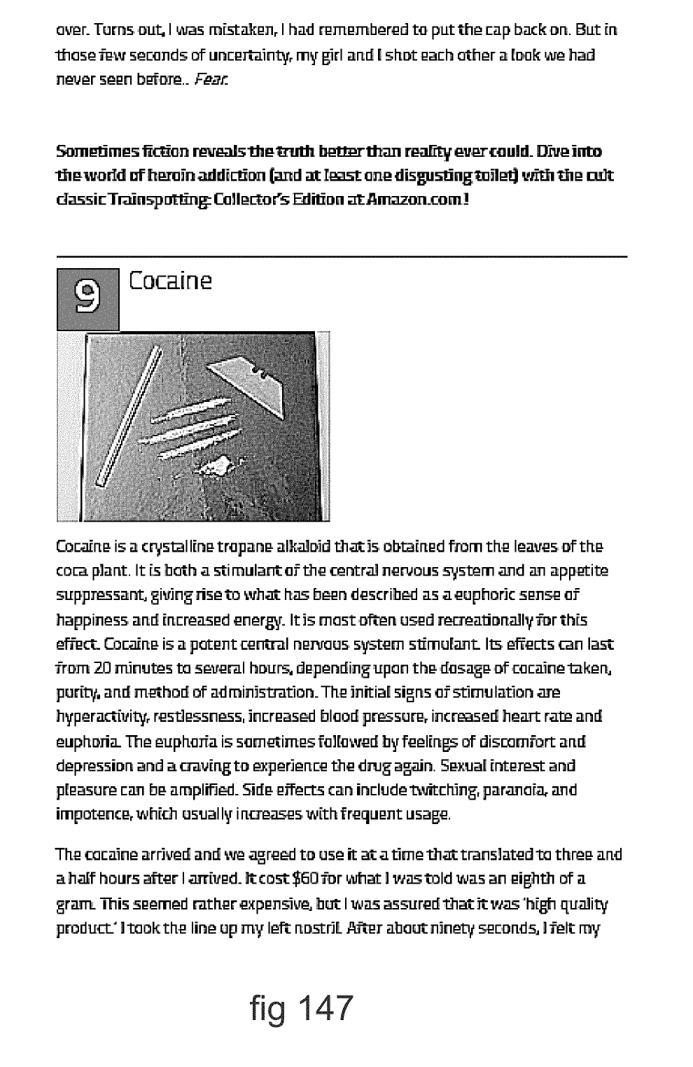

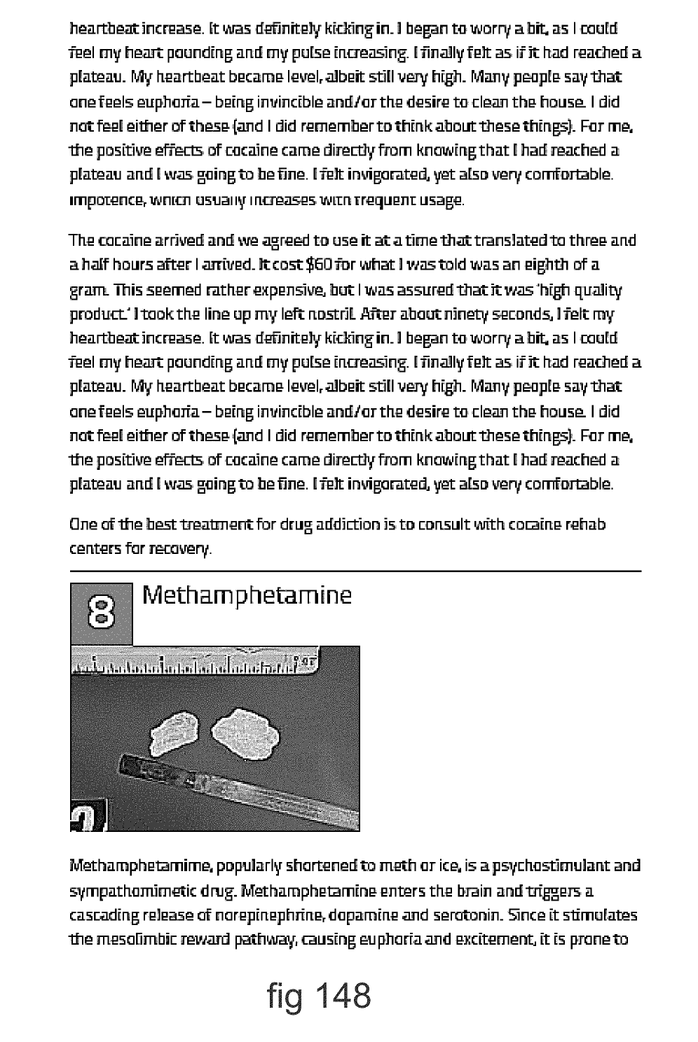

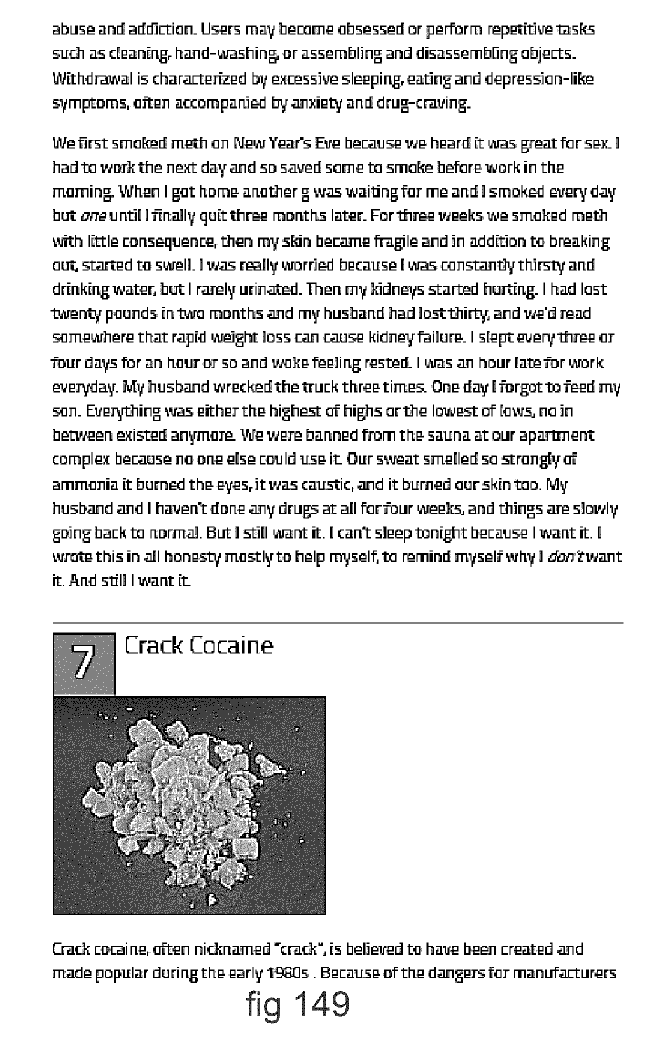

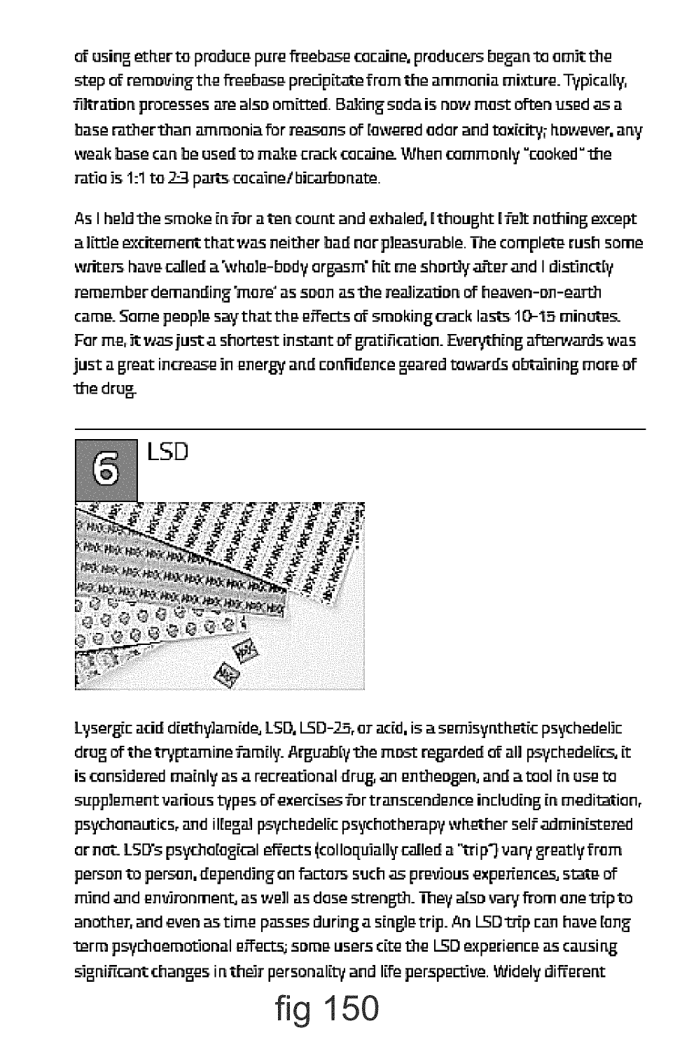

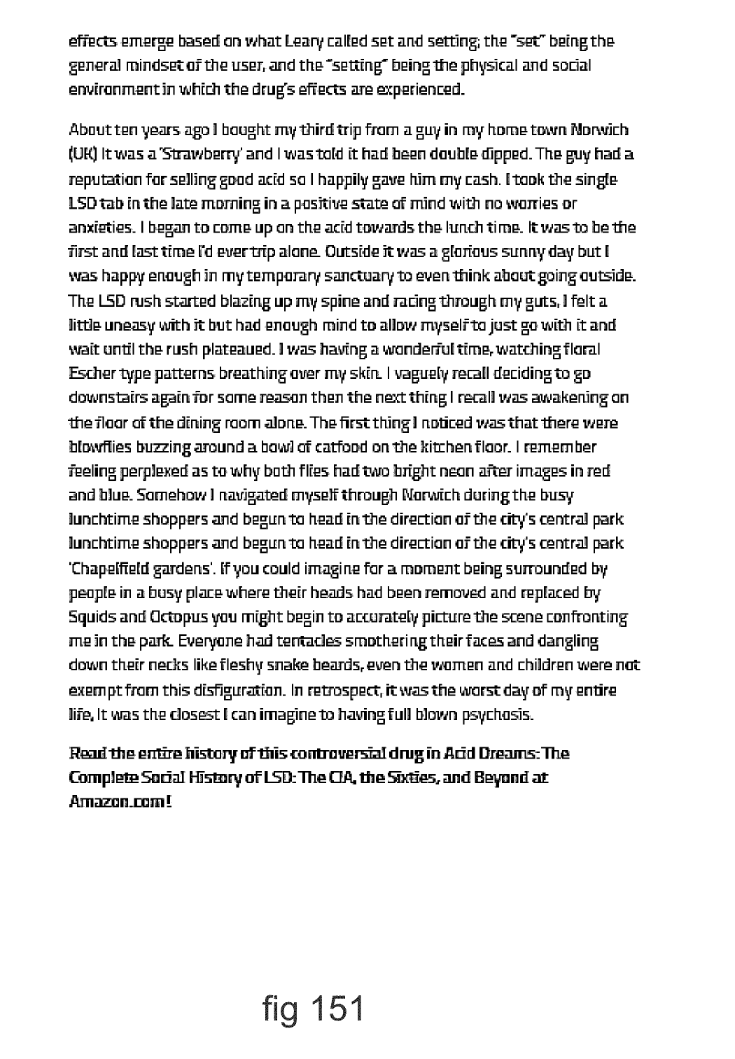

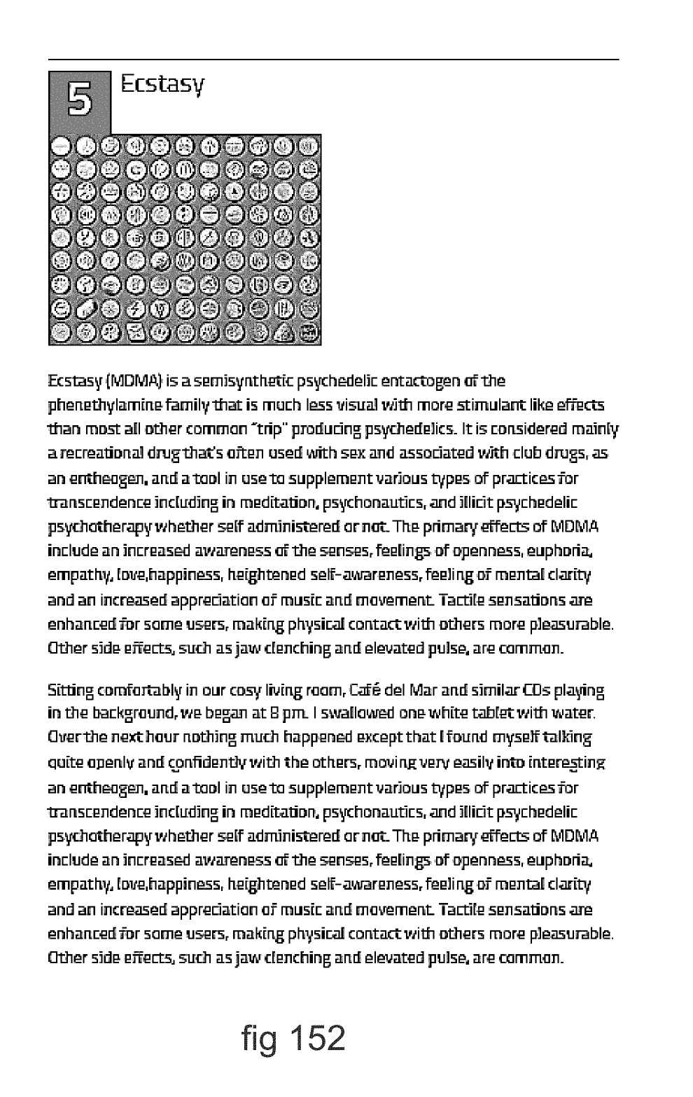

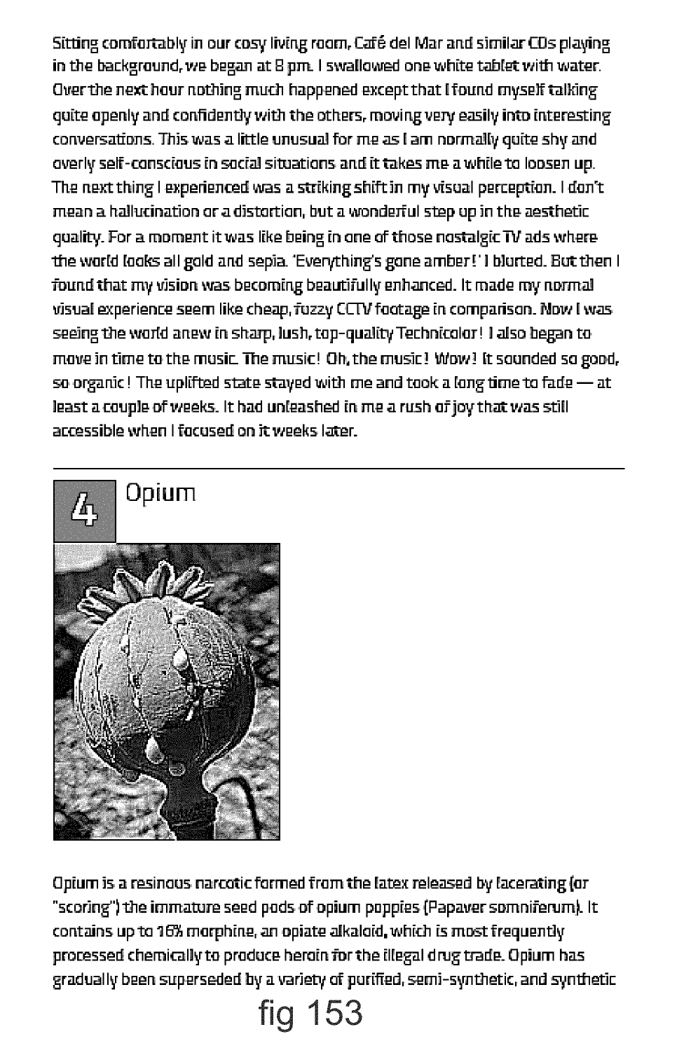

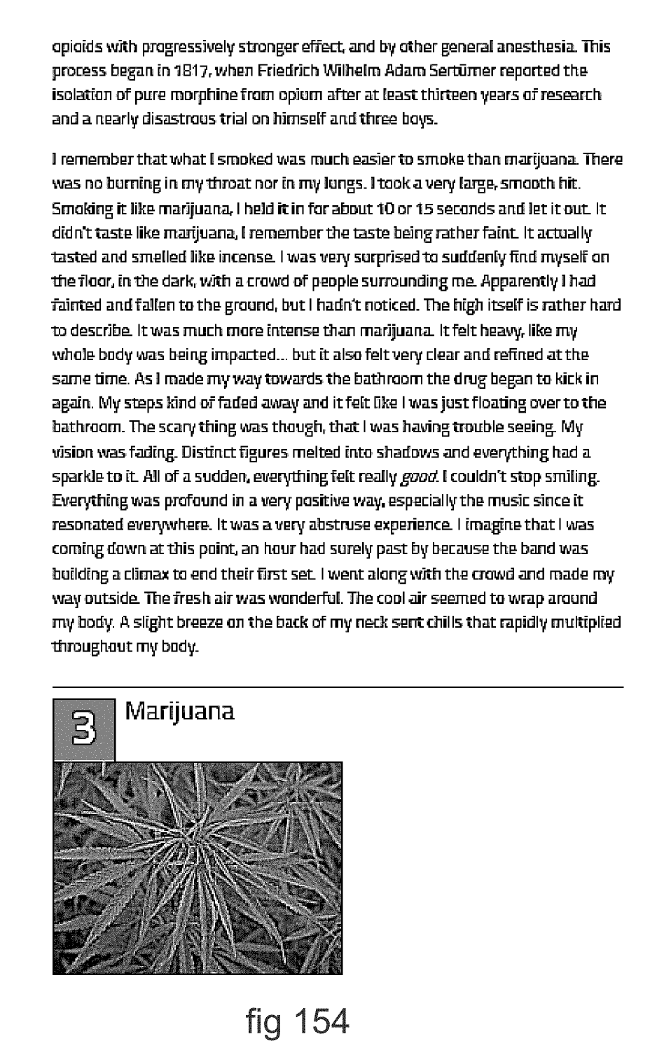

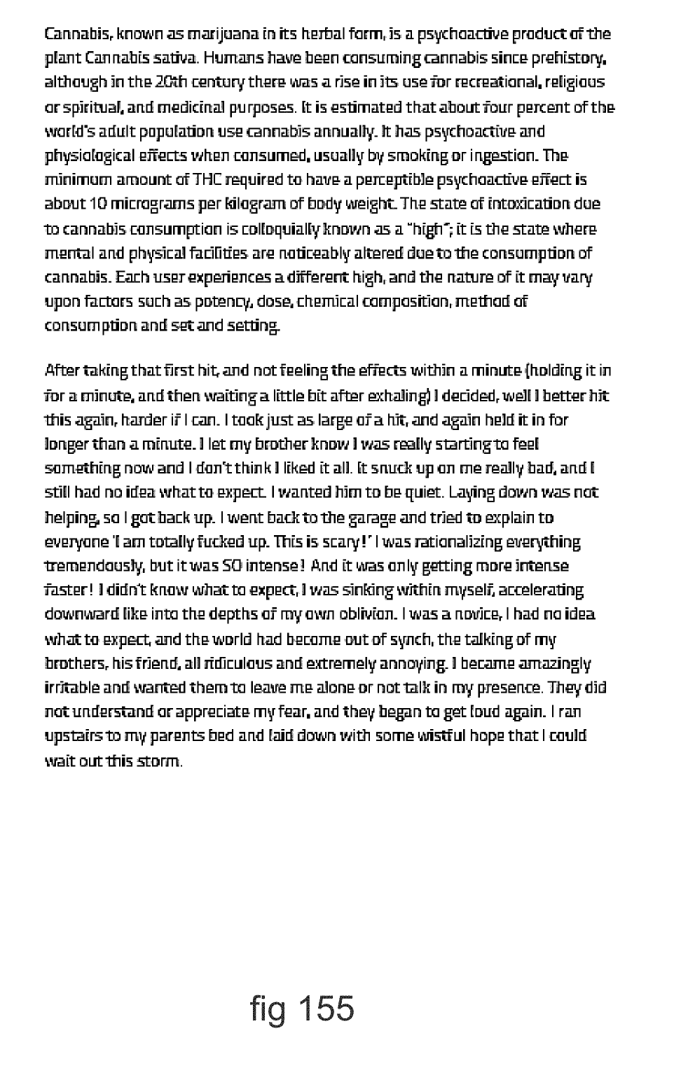

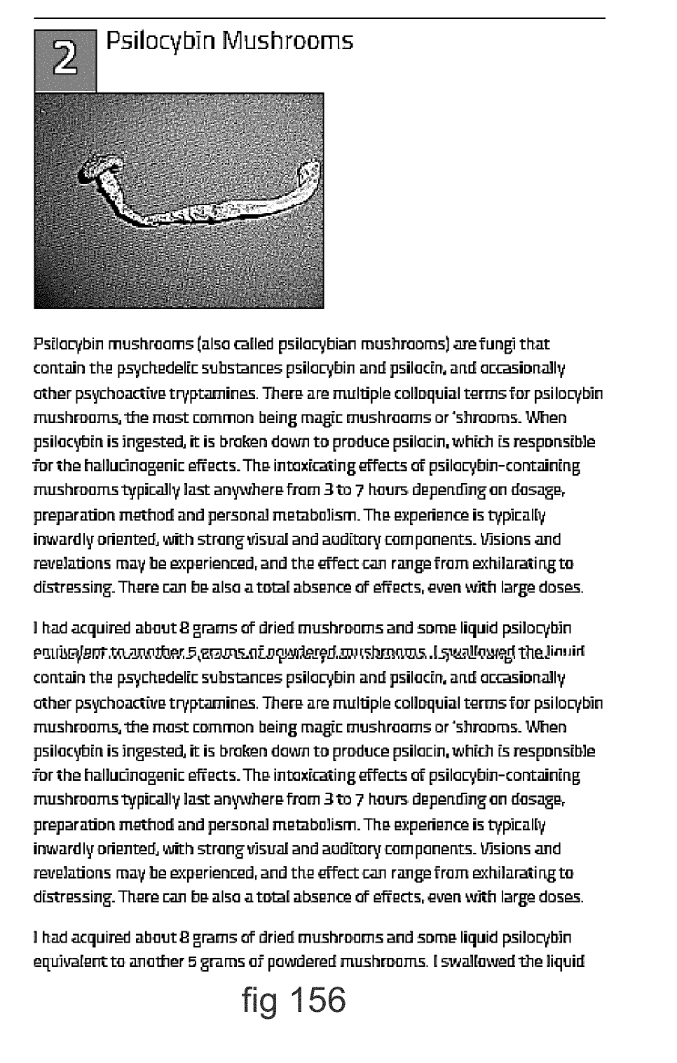

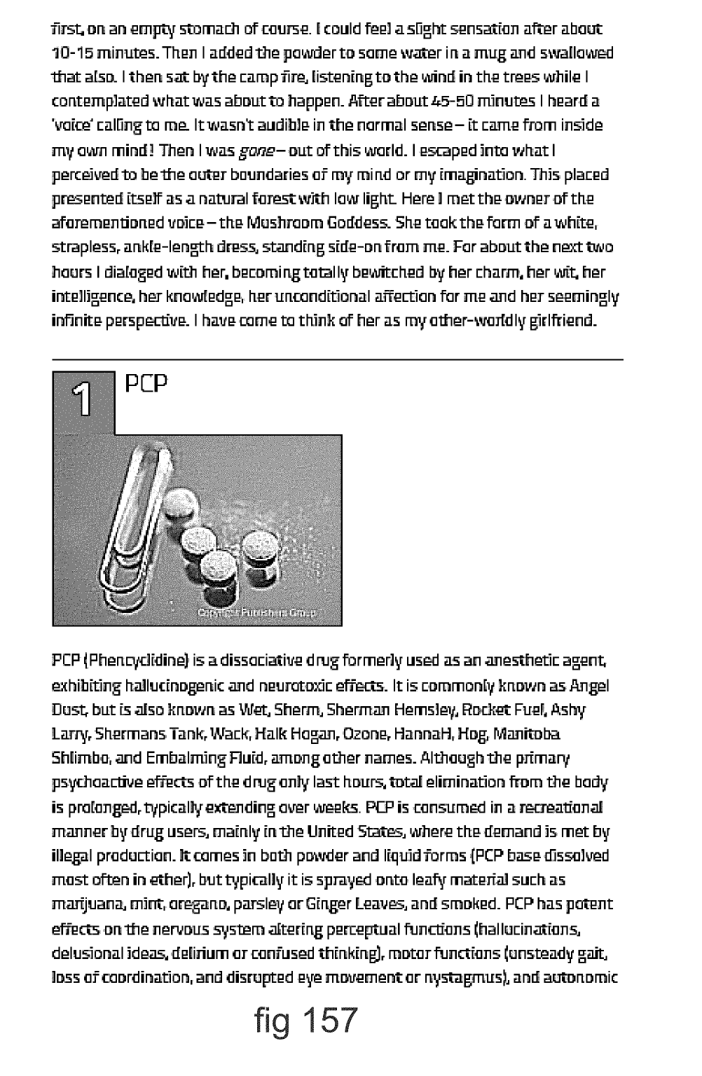

As shown best in FIG. 55, embodiment 212 in use is treated similar to a lollipop, and placed in the mouth with shaft 254 projecting outward. Distribution head 260 is very effective in delivering active ingredients when placed against the undersurface of tongue 266 (FIG. 55).

As shown best in FIGS. 52 and 53, Distribution head 260, and/or active ingredient collar 258 may be covered with skins 262 and 264 respectively, configured to control dissemination of active ingredients, as non-limiting and non-exhaustive examples, either by restriction, and/or by enhancing their delivery through contacted body surfaces through drugs, topography, and/or other means.

Skins 262 and 264 may only partially cover distribution head 260 and active ingredient collar 258. As a non-limiting and non-exhaustive example, only the underside of distribution head 260 might be covered by skin 262. This would allow unimpeded transmission of active ingredients to the underside of the tongue, while helping to retard transmission of active ingredients to other interior mouth surfaces.

Skins 262 and 264 might also allow for liquid or semi liquid active ingredients, by containing such active ingredients within an outer shell.

As shown best in FIG. 51, and as non-limiting and non-exhaustive examples, distribution head 260 may be of monolithic construction, without surrounding skin 262, with ingredients dissolving like a common lollipop in the saliva contained in the mouth. Alternatively, it may be constructed using a non-dissolving substrate which holds and releases active ingredients without its structure being degraded. Either dissolving or not, distribution head 260 may contain flavors, sweeteners, and/or colors to make it more attractive, similar to sucking on a lollipop.

Distribution head 260, and active ingredient collar 258 may contain dissimilar active ingredients, which, as already explained may be: complementary (cooperative) to, in opposition to, or disassociated from one another.

Active ingredient collar 258 may slide 256 along shaft 254 to position it closer or further from user nostrils 268 (best shown in FIG. 55). This allows a user to adjust intake amounts of active ingredients contained within active ingredient collar 258.

Embodiment 214 may utilize any suitable technology. As just one non-limiting and non-exhaustive example, it may use technologies similar to that used to make current

An example embodiment comprises lollipops. This may include using refined sugar, raw sugar, fructose, high fructose, and/or low or no calorie sweeteners, as well as colors, textures, and flavors.

As with all active ingredients herein transferred through the nose, various means of storing and distributing active ingredients may be used. As both non-limiting and non-exhaustive examples, proven Scratch and Sniff technology (see FIGS. 159 and 160) might be used, or even: cardboard, fabric, nonwoven material such as cotton, wool, felt, polyester, etc., or synthetic materials including plastic foams (including both open and/or closed cell foams, reticulated foams, self-skinning foams, rigid or resilient foams, other suitable foams, etc.).

Micro-encapsulation, such as employed in Scratch and Sniff technology, might also be used for transference of active ingredients through other means, including, but not necessarily limited to, the ear canal, nasal passages, orally, sublingually, trans-dermally, vaginally, anally, and/or through other suitable means. This may aid at least packaging and ease-of-use.

Active ingredient collar 258 may also be removed from shaft 254 simply by sliding it off the end of shaft 254. Similarly, active ingredient collar 258 may be easily replaced on shaft 254 with an alternative collar 258, to substitute active ingredients contained within active ingredient collar 258.

Being similar in appearance and function to a common lollipop helps make embodiment 214 more intuitive, easy, friendly and attractive to use.

FIG. 54 shows embodiment 214 in its packaged condition. Separated, compartments 270 and 272, may help keep active ingredients in distribution head 260 from interacting with active ingredients in active ingredient collar 258.

Shaft 254 may be constructed from non-permeable material, as a non-limiting and non-exhaustive example, ABS or polypropylene, in order to further aid in separating active ingredients in distribution head 260 from interacting with active ingredients in active ingredient collar 258.

Active ingredients contained within active ingredient collar 258 may instead be contained within shaft 254. To accomplish this, shaft 254 may be constructed from a material capable of holding and disseminating active ingredients. As a non-limiting and non-exhaustive example, it may be constructed from cardboard, similar to many common lollipops.

As with all embodiments herein, dosages may be dependent on several variables, which could include: desired outcome, targeted time period to achieve desired outcomes, user size, user age, user metabolism rate, user gender, other medications the user may be taking, etc.

When compared to existing active ingredient delivery devices, embodiments herein may offer specific advantages.

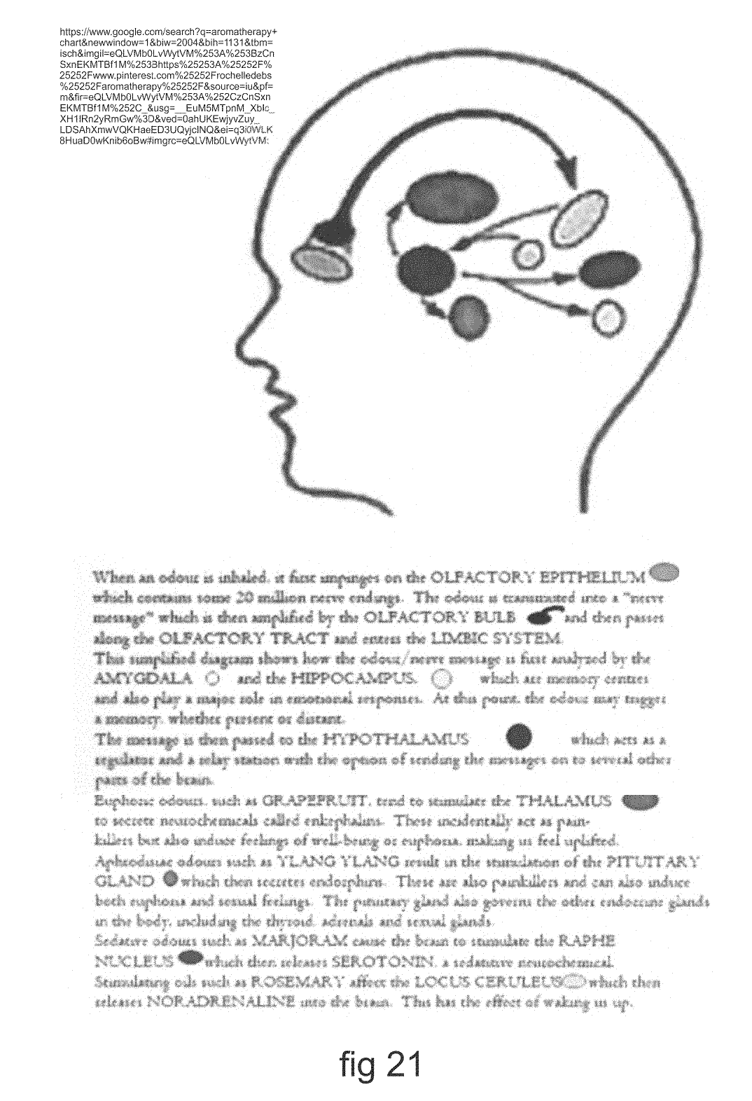

Fragrances, odors, and other substances delivered through olfactory and/or breathing means, such as shown in embodiments 214, 212, 210, 204, 180, 178, 176, and 116, may motivate primitive areas of the brain, and may quickly, as well as over time, evoke strong as well as fundamental reactions on many levels, both physically and emotionally (see FIG. 21 on one theory of how this works).

Combining olfactory and/or breathing transferred active ingredients, with active ingredients conveyed through other means may provide results which neither the olfactory and/or breathing transferred active ingredients nor active ingredients transferred through other means could achieve on their own.

Further, active ingredients transferred through sublingual means, such as are shown in embodiments 214, 212, 210, and 208, may be infused very rapidly into the bloodstream, when compared to orally ingested active ingredients.

As a particular non-limiting and non-exhaustive example, utilizing embodiment 112, in FIGS. 7 and 8, or other embodiments herein for delivery may offer advantages over using a skin patch, such as NICODERM patches currently used to help quit smoking. This is because surface layers in the nose, mouth, and ears, as well as in the anus and vagina, may offer less resistance to infusion of particular substances than epidermis found on other parts of the body.

One more non-limiting and non-exhaustive example of specific advantages embodiments herein may offer, embodiments herein which utilize active ingredients transference through ear canals, nasal passages, sublingual, transdermal, anal, and/or vaginally means, may bypass the digestive track, including the stomach and intestines. This may be advantageous, when, as a specific non-limiting and non-exhaustive example, active ingredients which may irritate the digestive tract, such as aspirin, are being transferred.

As another non-limiting and non-exhaustive example, utilizing embodiment 112, in FIGS. 7 and 8, as well as other embodiments herein, for inhaling active ingredient delivery, may provide a gentler longer inhalation infusion time, when compared to misting inhalers currently such as are currently used to treat acute asthma attacks. Additionally, such embodiments may be more convenient to use than current inhaling devices utilizing a facial mask to cover the nose and/or mouth.

In general, embodiments herein may infuse active ingredients over extended periods of time. This may be advantageous in evening out delivery rates.

As another non-limiting and non-exhaustive example, utilizing embodiment 112, as well as other embodiments herein, may offer a targeted and convenient way to deliver olfactory biological agents, such as utilized by practitioners of aromatherapy. In particular, it is not necessary, when using embodiments contained herein, to have a particular desired fragrance introduced to the entire room environment.

As another non-limiting and non-exhaustive example of embodiments herein having specific advantages over current drug delivery systems, when compared with transdermal patches, such as NICODERM, utilizing embodiments herein, such as embodiment 214, to help users quit smoking, embodiment 214 not only provides drug transference, like transdermal patches, but also satisfies oral needs, such as quitting smokers frequently have. It may be therefore more effective in the quit smoking treatment.

FIG. 161 gives non-limiting and non-exhaustive examples of potential embodiment applications: Embodiment shown herein may have many particular applications. FIG. 161 shows a non-limiting and non-exhaustive list of several such applications.

FIGS. 20 through 29, and FIGS. 56 through 160 show a non-limiting and non-exhaustive compilation of active ingredients which might be considered for use in embodiments herein.

A few specific examples may help to illustrate use of some of the embodiments contained herein.

As a non-limiting and non-exhaustive example, embodiment 106, may be constructed according to embodiment 128, with central orifice 152, but without interior tube 150. The basic body, including, but not limited to, reservoir 148, being constructed from an open cell poly foam, similar to that found in a fine cell poly foam kitchen sponge. Exterior surfaces 154 156 158 160 164 and 166 being coated with a flexible membrane which is less permeable to active ingredients than the interior surface of orifice 152.

Two such embodiments could be packaged in sealed envelope 172 according to FIG. 18, along with rosemary as an active ingredient which is stored in the basic body of embodiment of embodiment 128.

In use, an individual would:
tear open envelope 172,
remove the two embodiments 128,
insert them into their nostrils,
breathe normally for a prescribed period of time, and then
remove and dispose of the two embodiments 128.

This, according to the charts contained in FIGS. 20 through 29, would provide an invigorating effect.

Other alternative example embodiments include use of constituents of mucuna beans fir delivery, such as via cream, patch or lollipop as detailed above. This has been deemed useful in treatment of maladies such as restless leg syndrome. Also, constituents from Asian or Peruvian ginseng, horny goat weed zinc/chromium, tribulus or maca have been deemed useful in treatment of erectile dysfunction.

As another non-limiting and non-exhaustive example of a specific application, embodiment 106 could be constructed according to embodiment 112 in FIGS. 7 and 8, again using a fine open cell poly foam, packaged in sealed container 174 according to FIG. 19, along with the active ingredient Jasmine

In use, an individual would:
open sealed container 174
remove the two embodiments 112,
insert them into their nostrils,
breathe normally for a prescribed period of time, and then
remove and dispose of the two embodiments 112.

This, according to the charts contained in FIGS. 20 through 29, would provide an aphrodisiac effect.

Many variants and derivatives of embodiments herein would be obvious to one having ordinary skill in the art. Such variance and derivatives, while not specifically taught herein, should be considered as part of the teachings herein.

1. A drug delivery device comprising:
a plug configured for insertion into an ear canal and contacting inner surfaces thereof;
the plug further configured to transmit an associated drug from the plug, through inner surfaces of the ear canal, into the bloodstream.

2. The drug delivery device of claim 1, further including the plug configured with a cavity configured such that air freely passes through it when inserted into an ear canal.

3. The drug delivery device of claim 1, wherein the plug includes an orifice penetrating through it, and the orifice, when the plug is inserted into an ear canal, is configured to promote free passage of air through the plug, from an exterior of an ear into interior portions of the ear canal.

4. The drug delivery device of claim 2, further including the plug being constructed from open cell foam.

5. The drug deliveries device of claim 2, further including the plug being constructed from a fibrous material.

6. The drug delivery device of claim 1, further including the plug further configured to pass sound to freely when the plug is inserted into an ear canal.

7. The drug delivery device of claim 1, further including the plug being constructed from a generally flat sheet which is rolled into a tube.

8. The drug delivery device of claim 1, further including the plug being constructed of a resilient material.

9. The drug delivery device of claim 1, further including the plug having an end portion which is tapered inward toward its terminus, and the end portion being configured to be inserted into an ear canal.

10. A drug delivery device including:
a plug configured for insertion into a nostril and contacting inner surfaces of the nasal cavity posterior to the nostril;
a first drug carried by the plug, and configured to transmit from the plug, through inner surfaces of the nasal cavity, into the bloodstream.

11. The drug delivery device of claim 10, further including the plug further configured to promote free passage of air through it when inserted into a nostril.

12. The drug delivery device of claim 10, further including the plug having a disposed orifice, and the orifice, when the plug is inserted into a nostril, configured to allow air to pass through the plug, from the exterior of the nostril into interior portions of the nasal cavity.

13. The drug delivery device of claim 11, further including the plug being constructed from open cell foam.

14. The drug deliveries device of claim 11, further including the plug being constructed from a fibrous material.

15. The drug delivery device of claim 10, further including the plug being constructed from a generally flat sheet which is rolled into a tube.

16. The drug delivery device of claim 10, further including the plug being constructed of a resilient material.

17. The drug delivery device of claim 10, further including the plug having an end portion which is tapered inward toward its terminus, and the end portion being configured to be inserted into a nostril.

18. The drug delivery device of claim 10, wherein, when the plug is inserted into a nostril, the plug is configured to pass air from the exterior of the nostril, through the plug, to the nasal cavity posterior of the nostril, and the plug includes a reservoir holding a substance, and the reservoir configured to transmit the substance from the reservoir into air passing through the plug.

19. The drug delivery device of claim 18, wherein the reservoir is configured to hold a second drug which affects specific biological functions, and wherein the first drug effects specific biological functions, and at least some of the specific biological functions affected by the second drug are the same as the specific biological functions affected by the first drug.

20. The drug delivery device of claim 18, wherein the substance held by the reservoir is a second drug which affects specific biological functions, and the first drug affects specific biological functions, and at least some of the specific biological functions affected by the second drug are different than the specific biological functions affected by the first drug.

21. A drug delivery device, including:
an extensive member configured to extend into the mouth and be disposed against sublingual surfaces;
the extensive member holding a first drug, and being configured to transmit the first drug through sublingual surfaces into the bloodstream;
a reservoir configured to be disposed between upper and lower oral lips, and configured to hold a substance, and configured to transmit the substance into air passing from the exterior of the lips into the oral cavity;
the extensive member being coupled to the reservoir.

22. The drug delivery device of claim 21, further including instructions to insert the extensive member beneath the tongue.

23. A drug delivery device, including:
a first reservoir configured to be disposed against sublingual surfaces;
the first reservoir holding a first drug, and being configured to transmit the first drug through sublingual surfaces into the bloodstream;
a second reservoir configured to be disposed below a nostril, and configured to hold a substance, and configured to transmit the substance into air passing from the exterior of the nostril into the nasal cavity;
the first reservoir being coupled to the second reservoir.

24. The drug delivery device of claim 23 further including a restraint, configured to rest anterior of front teeth, and the restraint disposed intermediate of the first reservoir and the second reservoir, and the restraint being coupled to both the first reservoir and the second reservoir.

25. The drug delivery device claim 24, wherein there is a member connecting the first reservoir to second reservoir, and the restraint slides along the member.

26. A drug delivery device, including:
a first reservoir, holding a first drug and configured to be disposed sublingually;
the first reservoir configured to transfer the first drug, through sublingual surfaces and into the bloodstream;
a shaft projecting from the first reservoir;
a second reservoir, holding a substance and the second reservoir configured to be disposed along the shaft and below a nostril;
the second reservoir configured to transfer the substance into air passing into the nostril.

27. The drug delivery device of claim 26, wherein the first reservoir is configured so as to resemble the head of a lollipop.

28. The drug delivery device of claim 26, wherein the first reservoir is configured to hold an associated flavoring.

29. The drug delivery device of claim 26, further including the first reservoir configured so as to promote dissolving via saliva.

30. The drug delivery device of claim 26, further including the first reservoir configured so as to retard dissolving in saliva.

31. The drug delivery device of claim 26, wherein the second reservoir is configured to slide along the shaft.

32. The drug delivery device of claim 26, further including the shaft being constructed of material which is non-permeable to both the first drug and to the substance.

33. The drug delivery device of claim 26, further including a skin which is either semi-permeable or non-permeable to the first drug, and the skin covering at least a portion of the first reservoir.

34. The drug delivery device of claim 26, further including packaging which is constructed of material non-permeable to the first drug and non-permeable to the substance, and the packaging forming a sealed enclosure around the first reservoir, and the packaging forming a sealed enclosure around the second reservoir, and the packaging separating the first reservoir from the second reservoir.

35. The drug delivery device of claim 26, further including the substance being a drug.

36. The drug delivery device of claim 26, wherein the substance is a drug which is different than the first drug.

37. The drug delivery device of claim 26 further including instructions to place the first reservoir below the tongue.
